(19) United States
(12) Patent Application Publication
(10) Pub. No.: US 2026/0284240 A1
(43) Pub. Date: Sep. 24, 2026

(54) RADIOLABELED COMPOUND FOR USE IN A METHOD OF TREATING CARBONIC ANHYDRASE IX POSITIVE DISEASES

(71) Applicant: DEBIOPHARM INTERNATIONAL S.A., Lausanne (CH)

(72) Inventors: Ivana GALETIC, Allschwil (CH); Antoine ATTINGER, Colombier (CH); Frédéric MASSIERE, Lausanne (CH); Libuse TACHMANOVA, Milano (IT)

(21) Appl. No.: 19/495,317

(22) PCT Filed: Jun. 19, 2024
(86) PCT No.: PCT/EP2024/067154

§ 371 Date: Dec. 19, 2025

(30) Foreign Application Priority Data
Jun. 20, 2023 (EP) 23180304.0

(51) Int. Cl. A61K 51/08; A61K 9/00; A61P 35/00

(52) U.S. Cl. CPC A61K 51/088; A61K 9/0019; A61P 35/00

(57) ABSTRACT

The present invention relates to a radiolabeled compound for use in targeted radionuclide therapy. In particular, the present invention relates to a radiolabeled compound for use in a method of treating carbonic anhydrase IX (CAIX) positive diseases, which leads to improved delivery and therapeutic efficacy (antitumor activity).

The present invention relates to a radiolabeled compound for use in targeted radionuclide therapy. In particular, the present invention relates to a radiolabeled compound for use in a method of treating carbonic anhydrase IX (CAIX) positive diseases, which leads to improved delivery and therapeutic efficacy (antitumor activity).

### BACKGROUND OF THE INVENTION

Solid tumors often contain regions of hypoxia and/or acidosis. Hypoxia is a key factor of the tumor environment, primarily caused by aberrant vasculature. Hypoxia can induce a spectrum of cellular responses leading to cancer progression and treatment resistance (Harris Nat Rev Cancer 2002, 2 (1), 38-47).

Carbonic anhydrase IX (CAIX) is a tumor-associated, cell surface glycoprotein that is induced by hypoxia, involved in adaptation to acidosis, and implicated in cancer progression via its catalytic activity and/or non-catalytic functions (Pastorekova et al. Cancer Metastasis Rev 2019, 38(1-2), 65-77). CAIX belongs to the α-carbonic anhydrase family of zinc metalloenzymes, which catalyze the reversible hydration of carbon dioxide to bicarbonate ions and protons (Pastorek et al. Oncogene 1994, 9(10), 2877-88). This simple reaction is essential for virtually all biological processes that require acid-base balance in subcellular compartments and across the plasma membrane.

The dominant role of hypoxia in controlling CAIX expression is reflected by its presence in a broad range of solid tumors. The distribution of CAIX can be either diffuse or regional. Clear cell renal cell carcinoma (ccRCC) often carries an inactivating mutation/deletion of the von Hippel Lindau (VHL) tumor suppressor gene, resulting in constitutive activation of the hypoxia-inducible factor (HIF) pathway and expression of HIF-regulated genes, such as CAIX (Wiesener et al. Cancer Res 2001, 61(13), 5215-22). Due to this constitutive hypoxia-like response, CAIX is expressed in more than 90% of ccRCC and it can be detected in a high percentage of tumor cells (diffuse distribution) (Stillebroer et al. Eur Urol 2010, 58(1), 75-83).

In other types of cancers, the activation of the HIF pathway is caused by intra-tumoral hypoxia and CAIX expression is restricted to tumor regions that are hypoxic/acidic (regional distribution) and tends to increase with increasing tumor stage and grade (Wykoff et al. Cancer Res 2000, 60 (24), 7075-83). Notably, many tumors expressing CAIX show increased signs of cancer aggressiveness, and resistance to cancer treatments, including radiation, and are associated with tumor progression and poor prognosis (Stillebroer et al. Eur Urol 2010, 58(1), 75-83; van Kuijk et al. Front Oncol 2016, 6, 69).

Renal cell carcinoma (RCC) accounts for 5% and 3% of all cancers worldwide in men and women, respectively (Capitanio et al. Eur Urol 2019, 75(1), 74-84). Clear cell renal cell carcinoma (ccRCC) is the most common subtype (75-85% of RCC) and accounts for the majority of renal cancer-related deaths. Regardless of histology, 20-30% of patients who undergo surgical resection for local/loco-regional disease develop recurrence and 20-30% of newly diagnosed patients present de novo with advanced/metastatic disease (Saad et al. Clin Genitourin Cancer 2019, 17 (1): 46-57). Patients with advanced disease have a poor prognosis with an overall 5-year survival rate of less than 10%.

The incidence of colorectal cancer (CRC) is 29 per 100,000 in Western Europe and 26 per 100,000 in North America (Rawla et al. Prz Gastroenterol 2019, 14(2), 89-103). The 5-year OS for metastatic disease is 14% and there is a high unmet need for advanced metastatic disease. Recurrence occurs in 50% of stage III patients and 25% of stage II patients due to micrometastatic disease.

The incidence of pancreatic ductal adenocarcinoma (PDAC) is 8 per 100,000 in Western Europe and North America (Ushio et al Diagnostics (Basel) 2021, 11 (3)). It is the twelfth most common cancer worldwide and the seventh most common cause of cancer death. The 5-year OS is less than 10%. Surgery and chemotherapy improve survival in patients with early-stage disease, but PDAC is mostly diagnosed at a late stage and approximately 80% of patients are ineligible for curative surgery. In addition, immunotherapies have limited efficacy in PDAC due to the non-immunogenic and immunosuppressive microenvironment. Current treatment options for second-line therapy and beyond are limited and no standard of care has been defined. Therefore, there is a need for therapies with novel modes of action.

High expression of CAIX has been validated in several forms of cancer including, e.g., RCC, CRC and PDAC (Bui et al. Clin Cancer Res 2003, 9(2), 802-11; Courcier et al. Int J Mol Sci 2020, 21(19); Korkeila et al. Br J Cancer 2009, 100(6), 874-80; Juhasz et al. Aliment Pharmacol Ther 2003, 18(8), 837-46; Yang et al. Pathol Oncol Res 2018, 24 (4): 899-906; Strapcova et al. Cancers (Basel) 2020, 12(8); van Kuijk et al. Front Oncol 2016, 6, 69). Importantly, CAIX expression in non-cancerous (healthy) tissues is rare and generally restricted to the epithelia of the stomach, gallbladder, pancreas, and intestine (Pastorekova et al. Gastroenterology 1997, 112(2), 398-408).

The tumor expression of CAIX, complemented by a restricted profile in healthy tissues, provides an opportunity for therapeutic targeting and personalized medicine. It is therefore an object of the present invention to provide a compound for use in a method of treating a CAIX positive disease in a human patient, which achieves improved delivery and therapeutic efficacy (antitumor activity).

It is a further object of the present invention to provide a compound for use in a method of diagnosing and/or treating a CAIX positive disease in a human patient, which achieves improved delivery and therapeutic efficacy, while the side effects due to accumulation of radioactivity in non-cancerous (healthy) tissues are reduced or remain acceptable.

### SUMMARY OF THE PRESENT INVENTION

The present invention provides a compound for use in a method of treating CAIX positive diseases in a human patient. The present inventors have found that administering a treatment dose of a compound of the invention labeled with ¹⁷⁷Lu may lead to sufficient uptake of the radiolabeled compound in the tumor cells, while unspecific accumulation in healthy tissues (e.g., in the kidneys or other tissues endogenously expressing CAIX) is minimal/below an acceptable level. The compound labeled with ¹⁷⁷Lu may therefore have excellent therapeutic efficacy and minimal side effects. The inventors have also found that administering to human patients an imaging dose of a compound of the invention labeled with ⁶⁸Ga enables excellent imaging of the tumor tissues, thereby enabling the identification of patients that are likely to benefit from treatment with the compound of the invention labeled with ¹⁷⁷Lu.

The present invention thus relates to a compound for use in a method of treating a CAIX positive disease in a human patient comprising the steps of:
(i) optionally administering to the patient an imaging dose of the compound labeled with ⁶⁸Ga to obtain an image of body parts or tissues to be examined, and
(ii) administering to the patient a treatment dose of the compound labeled with ¹⁷⁷Lu to treat the CAIX positive disease;
   wherein the compound is represented by the following formula (1):

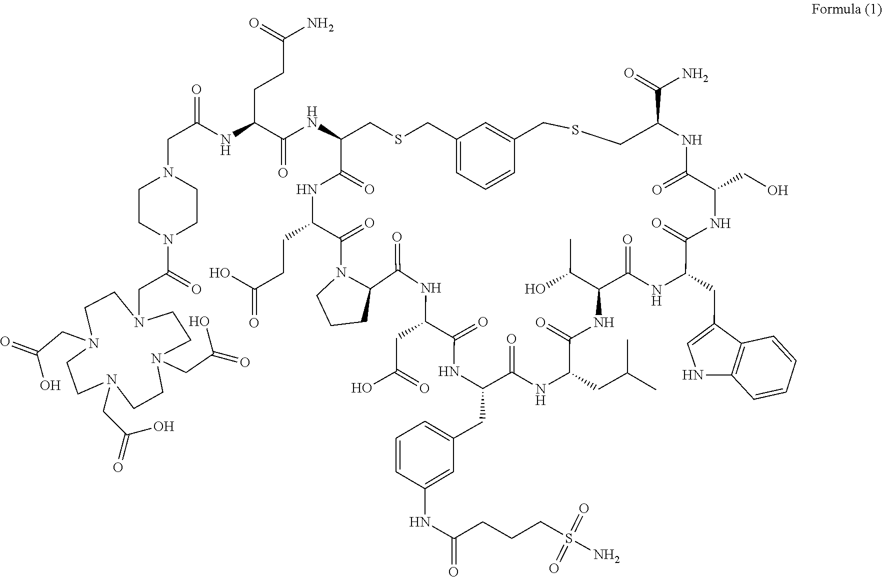 [SMILES: CC(C)C[C@@H]1NC(=O)[C@H](Cc2cccc(NC(=O)CCCS(N)(=O)=O)c2)NC(=O)[C@H](CC(=O)O)NC(=O)[C@H]2CCCN2C(=O)[C@H](CCC(=O)O)NC(=O)[C@@H](NC(=O)[C@H](CCC(N)=O)NC(=O)CN2CCN(C(=O)CN3CCN(CC(=O)O)CCN(CC(=O)O)CCN(CC(=O)O)CC3)CC2)CSCc2cccc(c2)CSC[C@@H](C(N)=O)NC(=O)[C@H](CO)NC(=O)[C@H](Cc2c[nH]c3ccccc23)NC(=O)[C@H]([C@@H](C)O)NC1=O]

wherein the DOTA moiety comprised in the compound of formula (1) chelates the ⁶⁸Ga or ¹⁷⁷Lu to form the compound labeled with ⁶⁸Ga or ¹⁷⁷Lu.

The present invention in particular includes the following embodiments (“Items”):
1. A compound for use in a method of treating a carbonic anhydrase IX (CAIX) positive disease in a human patient, the method comprising the steps of:
   (i) optionally administering to the patient an imaging dose of the compound labeled with ⁶⁸Ga, to obtain an image of body parts or tissues to be examined, and
   (ii) administering to the patient a treatment dose of the compound labeled with ¹⁷⁷Lu, to treat the CAIX-positive disease;
      wherein the compound is represented by the following Formula (1):

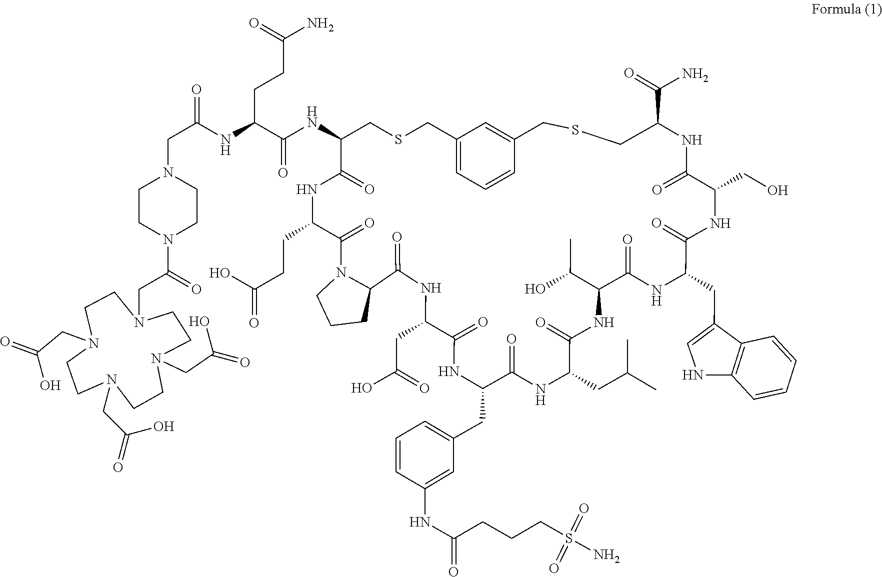 [SMILES: CC(C)C[C@@H]1NC(=O)[C@H](Cc2cccc(NC(=O)CCCS(N)(=O)=O)c2)NC(=O)[C@H](CC(=O)O)NC(=O)[C@H]2CCCN2C(=O)[C@H](CCC(=O)O)NC(=O)[C@@H](NC(=O)[C@H](CCC(N)=O)NC(=O)CN2CCN(C(=O)CN3CCN(CC(=O)O)CCN(CC(=O)O)CCN(CC(=O)O)CC3)CC2)CSCc2cccc(c2)CSC[C@@H](C(N)=O)NC(=O)[C@H](CO)NC(=O)[C@H](Cc2c[nH]c3ccccc23)NC(=O)[C@H]([C@@H](C)O)NC1=O]

wherein the DOTA moiety comprised in the compound of Formula (1) chelates the ⁶⁸Ga or ¹⁷⁷Lu to form the compound labeled with ⁶⁸Ga or ¹⁷⁷Lu,
and wherein, if the human patient is not administered an imaging dose of the compound labeled with ⁶⁸Ga in step (i), the compound labeled with ¹⁷⁷Lu administered in step (ii) is administered once per cycle of one to six weeks, preferably once per cycle of four to six weeks, and most preferably once per cycle of four weeks.
2. The compound for use according to item 1, wherein said method comprises the steps of:
   (i) administering to the patient an imaging dose of the compound labeled with ⁶⁸Ga, to obtain an image of body parts or tissues to be examined, and
   (ii) administering to the patient a treatment dose of the compound labeled with ¹⁷⁷Lu, to treat the CAIX positive disease.
   3. The compound for use according to item 1 or 2, wherein
(a) the imaging dose of the compound labeled with ⁶⁸Ga is from 50 to 250 MBq, preferably from 100 to 200 MBq, more preferably from 145 to 225 MBq, such as about 185 MBq, and/or
   (b) the treatment dose of the compound labeled with ¹⁷⁷Lu is from 1.0 to 25.0 GBq, preferably from 2.0 to 20.0 GBq, more preferably from 3.0 to 19 GBq.
4. The compound for use according to item 1, 2 or 3, wherein the compound labeled with ¹⁷⁷Lu is administered once per cycle of one to six weeks, preferably once per cycle of four to six weeks, and most preferably once per cycle of four weeks.
5. The compound for use according to any of items 1 to 4, wherein the compound labeled with ¹⁷⁷Lu-labeled is administered over one to ten cycles, preferably over four to eight cycles, and more preferably over four to six cycles.
6. The compound for use according to any of items 1 to 5, wherein the treatment dose of the compound labeled with ¹⁷⁷Lu is selected from the following:
   (1) a treatment dose of from 2.0 to 6.0 GBq, such as about 3.7 GBq,
   (2) a treatment dose of from 6.0 to 10.0 GBq, such as about 7.4 GBq,
   (3) a treatment dose of from 10.0 to 14.0 GBq, such as about 11.1 GBq,
   (4) a treatment dose of from 14.0 to 18.0 GBq, such as about 14.8 GBq, and
   (5) a treatment dose of from 18.0 to 20.0 GBq, such as about 18.5 GBq.
7. The compound for use according to any of items 1 to 6, wherein the compound labeled with ⁶⁸Ga and/or the compound labeled with ¹⁷⁷Lu is administered intravenously and preferably intravenously by infusion.
8. The compound for use according to any of items 1 to 7 wherein the compound labeled with ⁶⁸Ga and/or the compound labeled with ¹⁷⁷Lu is provided as a solution in a pharmaceutically acceptable injectable carrier.
9. The compound for use according to item 8, wherein the solution of the compound labeled with ⁶⁸Ga has a concentration of from 250 to 950 MBq/8.1 mL, and the solution of the compound labeled with ¹⁷⁷Lu has a concentration of from 150 to 900 MBq/mL.
10. The compound for use according to any of items 1 to 9, wherein the CAIX positive disease is cancer, and preferably the human patient has unresectable, locally advanced, or metastatic solid tumors.
11. The compound for use according to any of items 1 to 10, wherein the CAIX-positive disease is a cancer selected from the group consisting of renal cell carcinoma (RCC), in particular clear cell renal cell carcinoma (ccRCC), colorectal carcinoma (CRC), pancreatic ductal adenocarcinoma (PDAC), glioblastoma (GBM), mesothelioma, cholangiocarcinoma (CCA), ovarian carcinoma, non-small cell lung cancer (NSCLC), in particular squamous non-small cell lung cancer (SNSCLC), brain cancer, pancreatic cancer, thyroid cancer, lung cancer, renal cancer, breast cancer, in particular triple-negative breast cancer (TNBC), head and neck cancer, in particular squamous carcinoma of head and neck (SCCHN), urothelial carcinoma and bladder cancer.
12. The compound for use according to any of items 1 to 11, wherein the CAIX positive disease is a cancer selected from the group consisting of ccRCC, CRC, PDAC, SNSCLC, TNBC and SCCHN, and preferably from ccRCC, CRC and PDAC.
13. The compound for use according to any of items 1 to 12, wherein the compound chelating ⁶⁸Ga and/or the compound chelating ¹⁷⁷Lu is administered after one or more other therapeutic agents or therapies, such as DNA damage response (DDR) inhibitors, chemotherapeutic agents, immunomodulatory agents, proton pump inhibitors (PPIs), histamine H₂-receptor antagonists, tyrosine kinase inhibitors, cell therapy, external beam radiation, or any other targeted therapies.

In one aspect, the present invention includes the following embodiments (“Items”):
1. A compound for use in a method of treating a carbonic anhydrase IX (CAIX) positive disease in a human patient, the method comprising the steps of:
   (i) administering to the patient an imaging dose of the compound labeled with ⁶⁸Ga, to obtain an image of body parts or tissues to be examined, and
   (ii) administering to the patient a treatment dose of the compound labeled with ¹⁷⁷Lu, to treat the CAIX positive disease,
      wherein the compound is represented by the following Formula (1):

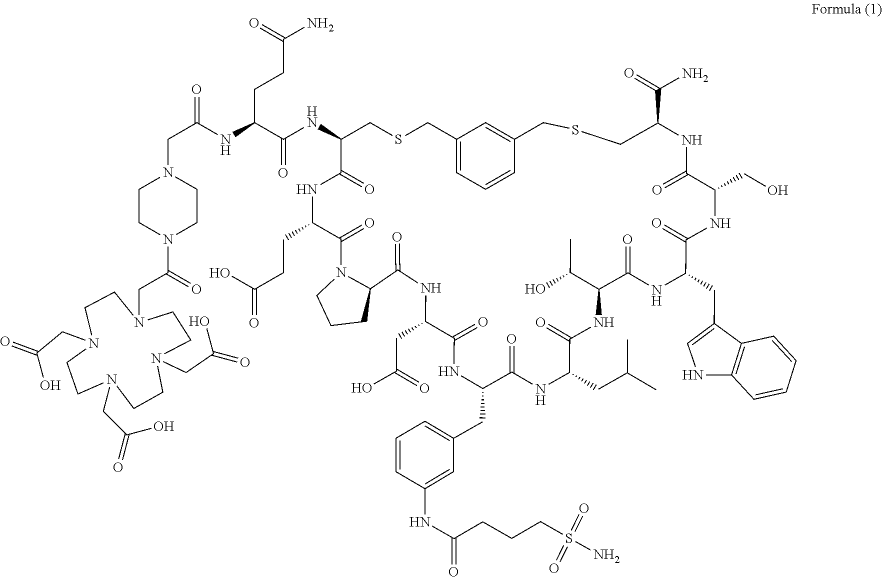 [SMILES: CC(C)C[C@@H]1NC(=O)[C@H](Cc2cccc(NC(=O)CCCS(N)(=O)=O)c2)NC(=O)[C@H](CC(=O)O)NC(=O)[C@H]2CCCN2C(=O)[C@H](CCC(=O)O)NC(=O)[C@@H](NC(=O)[C@H](CCC(N)=O)NC(=O)CN2CCN(C(=O)CN3CCN(CC(=O)O)CCN(CC(=O)O)CCN(CC(=O)O)CC3)CC2)CSCc2cccc(c2)CSC[C@@H](C(N)=O)NC(=O)[C@H](CO)NC(=O)[C@H](Cc2c[nH]c3ccccc23)NC(=O)[C@H]([C@@H](C)O)NC1=O]

wherein the DOTA moiety comprised in the compound of Formula (1) chelates the ⁶⁸Ga or ¹⁷⁷Lu to form the compound labeled with ⁶⁸Ga or ¹⁷⁷Lu.
2. The compound for use according to claim 1, wherein
   (a) the imaging dose of the compound labeled with ⁶⁸Ga is from 50 to 250 MBq, preferably from 100 to 200 MBq, more preferably from 145 to 225 MBq, such as about 185 MBq, and/or
   (b) the treatment dose of the compound labeled with ¹⁷⁷Lu is from 1.0 to 25.0 GBq, preferably from 2.0 to 20.0 GBq, more preferably from 3.0 to 19 GBq.
3. The compound for use according to claim 1 or 2, wherein the compound labeled with ¹⁷⁷Lu is administered once per cycle of one to six weeks, preferably once per cycle of four to six weeks, and most preferably once per cycle of four weeks.
4. The compound for use according to any of claims 1 to 3, wherein the compound labeled with ¹⁷⁷Lu-labeled is administered over one to ten cycles, preferably over four to eight cycles, and more preferably over four to six cycles.
5. The compound for use according to any of claims 1 to 4, wherein the treatment dose of the compound labeled with ¹⁷⁷Lu that is administered in at least one cycle, and preferably in each cycle, is selected from the following:
   (1) a treatment dose of from 2.0 to 6.0 GBq, such as about 3.7 GBq,
   (2) a treatment dose of from 6.0 to 10.0 GBq, such as about 7.4 GBq,
   (3) a treatment dose of from 10.0 to 14.0 GBq, such as about 11.1 GBq,
   (4) a treatment dose of from 14.0 to 18.0 GBq, such as about 14.8 GBq, and
   (5) a treatment dose of from 18.0 to 20.0 GBq, such as about 18.5 GBq.
6. The compound for use according to any of claims 1 to 5, wherein the compound labeled with ⁶⁸Ga and/or the compound labeled with ¹⁷⁷Lu is administered intravenously, and preferably intravenously by infusion.
7. The compound for use according to any of claims 1 to 6, wherein the compound labeled with ⁶⁸Ga and/or the compound labeled with ¹⁷⁷Lu is provided as a solution in a pharmaceutically acceptable injectable carrier.
8. The compound for use according to claim 7, wherein the solution of the compound labeled with ⁶⁸Ga has a concentration of from 250 to 950 MBq/8.1 mL, and the solution of the compound labeled with ¹⁷⁷Lu has a concentration of from 150 to 900 MBq/mL.
9. The compound for use according to any of claims 1 to 8, wherein the CAIX positive disease is cancer, and preferably the human patient has unresectable, locally advanced, or metastatic solid tumors.
10. The compound for use according to any of claims 1 to 9, wherein the CAIX positive disease is a cancer selected from the group consisting of renal cell carcinoma (RCC), in particular clear cell renal cell carcinoma (ccRCC), colorectal carcinoma (CRC), pancreatic ductal adenocarcinoma (PDAC), glioblastoma (GBM), mesothelioma, cholangiocarcinoma (CCA), ovarian carcinoma, non-small cell lung cancer (NSCLC), in particular squamous non-small cell lung cancer (SNSCLC), brain cancer, pancreatic cancer, thyroid cancer, lung cancer, renal cancer, breast cancer, in particular triple-negative breast cancer (TNBC), head and neck cancer, in particular squamous carcinoma of head and neck (SCCHN), urothelial carcinoma and bladder cancer.
11. The compound for use according to any of claims 1 to 10, wherein the CAIX positive disease is a cancer selected from the group consisting of ccRCC, CRC, PDAC, SNSCLC, TNBC and SCCHN, and preferably from ccRCC, CRC and PDAC.
12. The compound for use according to any of claims 1 to 11, wherein the compound labeled with ⁶⁸Ga and/or the compound labeled with ¹⁷⁷Lu is administered after one or more other therapeutic agents or therapies, such as DNA damage response (DDR) inhibitors, chemotherapeutic agents, immunomodulatory agents, proton pump inhibitors (PPIs), histamine H₂-receptor antagonists, tyrosine kinase inhibitors, cell therapy, external beam radiation, or any other targeted therapies.

### FIGURES

FIG. 1—In vivo efficacy in terms of tumor volume (A), impact on relative body weight (B) and tumor uptake (C) of ¹⁷⁷Lu-DPI-4452 in the HT-29 xenograft mouse model.

FIG. 2—In vivo uptake of ¹⁷⁷Lu-DPI-4452 in kidneys (A) and liver (B) as well as the comparison of the uptake of ¹⁷⁷Lu-DPI-4452 and ⁶⁸Ga-DPI-4452 in kidney, liver, and tumor (C) in the HT-29 xenograft mouse model.

FIG. 3—In vivo images of intravenously injected ⁶⁸Ga-DPI-4452 at 1 hour p.i. and ¹⁷⁷Lu-DPI-4452 at 4 hours p.i. in the HT29 xenograft mouse model. Representative axial, coronal and maximal intensity (bottom) projection (MIP) images of two mice are shown (FIG. 17A: first mouse, FIG. 17B: second mouse). Uptake is presented as percent injected dose per gram tissue (% ID/g).

FIG. 4—In vivo efficacy in terms of tumor volume (A), impact on relative body weight (B) and tumor uptake (C) of ¹⁷⁷Lu-DPI-4452 in the SK-RC-52 xenograft mouse model.

FIG. 5—In vivo uptake of ¹⁷⁷Lu-DPI-4452 in kidneys (A) and liver (B) as well as the comparison of the uptake of ¹⁷⁷Lu-DPI-4452 and ⁶⁸Ga-DPI-4452 in kidney, liver and tumor (C) in the SK-RC-52 xenograft mouse model.

FIG. 6—In vivo imaging of ¹⁷⁷Lu-DPI-4452 in the SK-RC-52 xenograft mouse model. Representative axial, coronal and maximal intensity (bottom) projection (MIP) images for two mice are shown. Uptake is presented as percent injected dose per gram tissue (% ID/g).

FIG. 7—In vivo hematological analysis results following administration of ¹⁷⁷Lu-DPI-4452 to HT-29-xenografted mice. The X-axis represents the study day post injection. QW indicates the weekly dosing regimen.

FIG. 8—In vivo hematological analysis results following administration of ¹⁷⁷Lu-DPI-4452 to SK-RC-52-xenografted mice. The X-axis represents the study day post injection. QW indicates the weekly dosing regimen.

FIG. 9—In vivo creatinine (pmol/L) and urea (mmol(L) levels following administration of ¹⁷⁷Lu-DPI-4452 to SK-RC-52-xenografted mice. The X-axis represents the study day post injection. QW indicates the weekly dosing regimen.

FIG. 10—Inclusion based on SK-RC-52 tumor volume and body weight at the day of dosing. No significant difference in tumor volume (p=0.80, one-way ANOVA) or body weight (p=0.96, one-way ANOVA) was observed between the groups. N=3/group, Mean±SEM.

FIG. 11—Inclusion based on HT-29 tumor volume and body weight at day−1 (the day before dosing). No significant difference in tumor volume (p=0.80, unpaired t-test) or body weight (p=0.32, unpaired t-test) was observed between the groups. N=3/group, Mean±SEM.

FIG. 12—Representative SPECT/CT images (axial, coronal and maximum intensity projection images) of one mouse from group A1 at 1, 4, 24 and 48 hours post injection of [¹¹¹In]In-DPI-4452.

FIG. 13—Representative SPECT/CT images (axial, coronal and maximum intensity projection images) of one mouse from group A2 at 1, 4, 24 and 48 hours post injection of [¹¹¹In]In-DPI-4501.

FIG. 14—Representative SPECT/CT images (axial, coronal and maximum intensity projection images) of one mouse from group A3 at 1, 4, 24 and 48 hours post injection of [¹¹¹In]In-DPI-4452+gelofusine.

FIG. 15—Representative SPECT/CT images (axial, coronal and maximum intensity projection images) of one mouse from group A4 at 1, 4, 24 and 48 hours post injection of [¹¹¹In]In-DPI-4501+gelofusine.

FIG. 16—Representative SPECT/CT images (axial, coronal and maximum intensity projection images) of one mouse from group B1 at 2, 4, 24 and 48 hours post injection of [¹¹¹In]In-DPI-4452.

FIG. 17—Representative SPECT/CT images (axial, coronal and maximum intensity projections images) of one mouse from group B2 at 2, 4, 24 and 48 hours post injection of [¹¹¹In]In-DPI-4501.

FIG. 18—Percentage of injected dose per gram of tissue (% ID/g) uptake of [¹¹¹In]In-DPI-4452 and [¹¹¹In]In-DPI-4501 in SK-RC-52 and HT-29 tumor, kidneys, liver and blood. Uptake in the SK-RC-52 tumor mouse model was compared with injection of gelofusine immediately before injection of compounds. N=3/group, Mean±SEM.

FIG. 19—Percentage of injected dose per gram of tissue (% ID/g) uptake of [¹¹¹In]In-DPI-4452 and [¹¹¹In]In-DPI-4501 in SK-RC-52 and HT-29 tumor, kidneys, liver and blood (logarithmic scale). Uptake in the SK-RC-52 tumor model was compared with injection of gelofusine immediately before injection of compounds. N=3/group, Mean±SEM.

FIG. 20—[¹¹¹In]In-DPI-4452 versus [¹¹¹In]In-DPI-4501 pharmacokinetics in dog blood (% ID/g). Activity measurement in ex vivo blood samples from males (left) and females (right) after injection with In-111-labeled DPI-4452 and In-111-labeled DPI-4501. Y-axis presented in log 10. N=2/group. Plots represent mean±SEM.

FIG. 21—Male versus female dog pharmacokinetics in blood (% ID/g). Activity measurement in ex vivo blood samples from males and females after injection with In-111-labeled DPI-4452 (left) and In-111-labeled DPI-4501 (right), respectively. Y-axis presented in log 10. N=2/group. Plots represent mean±SEM.

FIG. 22—[¹¹¹In]In-DPI-4452 versus [¹¹¹In]In-DPI-4501 pharmacokinetics in dog urine (% ID/g). Activity measurement in ex vivo urine samples from males (left) and females (right) after injection with In-111-labeled DPI-4452 and In-111-labeled DPI-4501. Y-axis presented in log 10. N=2/group. Plots represent mean±SEM.

FIG. 23—Male versus female dog pharmacokinetics in urine (% ID/g). Activity measurement in ex vivo urine samples from males and females after injection with respectively In-111-labeled DPI-4452 (left) and In-111-labeled DPI-4501 (right). Y-axis presented in log 10. N=2/group. Plots represent mean±SEM.

FIG. 24—SPECT/CT-derived biodistribution data of [¹¹¹In]In-DPI-4452 (% ID/g and SUV) in male and female dogs. Graphs represent imaging time points of 1 h (left), 4 h (middle), and 48 h (right) post injection, respectively. X-axis present the investigated organs. N=2/group (N=1 in female 4 h scan group). Plots represent mean±SEM.

FIG. 25—SPECT/CT-derived biodistribution data of [¹¹¹In]In-DPI-4501 (% ID/g and SUV) in male and female dogs. Graphs represent imaging time points of 1 h (left), 4 h (middle), and 48 h (right) post injection, respectively. X-axis present the investigated organs. N=2/group. Plots represent mean±SEM.

FIG. 26—Representative SPECT/CT images of [¹¹¹In]In-DPI-4452 biodistribution in female dogs. Scan images of one female beagle dog at respectively 1 hour, 4 hours and 48 hours after injection. Scalebar represents SUV values.

FIG. 27—Representative SPECT/CT images of [¹¹¹In]In-DPI-4452 biodistribution in male dogs. Scan images of one male beagle dog at respectively 1 hour, 4 hours and 48 hours after injection. Scalebar represents SUV values.

FIG. 28—Representative SPECT/CT images of [¹¹¹In]In-DPI-4501 biodistribution in female dogs. Scan images of one female beagle dog at respectively 1 hour, 4 hours and 48 hours after injection. Scalebar represents SUV values.

FIG. 29—Representative SPECT/CT images of [¹¹¹In]In-DPI-4501 biodistribution in male dogs. Scan images of one male beagle dog at respectively 1 hour, 4 hours and 48 hours after injection. Scalebar represents SUV values.

FIG. 30—Mean total plasma concentration of DPI-4452 versus time profiles following a single i.v. bolus injection of 25, 80, 400 and 800 μg/kg DPI-4452 in male beagle dogs. N=6/group, Mean±SD.

FIG. 31—Mean total plasma concentration of 16, 80, and 400 μg/kg DPI-4452 versus time profiles following a single i.v. bolus injection of DPI-4452 in beagle dogs. N=2, Mean±SD.

FIG. 32A—PET image of right renal metastasis in a patient with metastatic ccRCC (after a single 185 MBq dose of Ga⁶⁸-DPI-4452).

FIG. 32B—PET image of pulmonary metastasis in a patient with metastatic ccRCC (after a single 185 MBq dose of Ga⁶⁸-DPI-4452).

FIG. 33—PET imaging data in patient with metastatic ccRCC (at 4 time points after single 185 MBq dose of Ga⁶⁸-DPI-4452).

FIG. 34A—Lung metastasis PET imaging data in patient with metastatic ccRCC (at 5 time points after single 185 MBq dose of Ga⁶⁸-DPI-4452).

FIG. 34B—Adrenal metastasis PET imaging data in patient with metastatic ccRCC (at 5 time points after single 185 MBq dose of Ga⁶⁸-DPI-4452).

FIG. 35A—Gastric uptake (healthy organ) PET imaging data in patient with metastatic ccRCC (at 5 time points after single 185 MBq dose of Ga⁶⁸-DPI-4452).

FIG. 35B—Small bowel uptake (healthy organ) PET imaging data in patient with metastatic ccRCC (at 4 time points after single 185 MBq dose of Ga⁶⁸-DPI-4452).

FIG. 36A—Bladder uptake and excretion PET imaging data in patient with metastatic ccRCC (at 5 time points after single 185 MBq dose of Ga⁶⁸-DPI-4452).

FIG. 36B—Renal uptake PET imaging data in patient with metastatic ccRCC (at 5 time points after single 185 MBq dose of Ga⁶⁸-DPI-4452).

FIG. 37A—PET imaging data in patient with metastatic ccRCC (at 2 time points after single 185 MBq dose of Ga⁶⁸-DPI-4452).

FIG. 37B—PET imaging data in patient with metastatic ccRCC (after single 185 MBq dose of Ga⁶⁸-DPI-4452).

FIG. 38—Right Parotid metastasis imaging data in patient with metastatic ccRCC (at 4 time points after single 185 MBq dose of Ga⁶⁸-DPI-4452).

FIG. 39—Lung metastasis imaging data in patient with metastatic ccRCC (at 4 time points after single 185 MBq dose of Ga⁶⁸-DPI-4452).

FIG. 40—Gastric and small bowel uptake (healthy organ) PET imaging data in patient with metastatic ccRCC (at 4 time points after single 185 MBq dose of Ga⁶⁸-DPI-4452).

FIG. 41—Tumor and gastrointestinal uptake (healthy organ) PET imaging data in Patient (3) with metastatic PDAC (1 hr after a single 185 MBq dose of Ga⁶⁸-DPI-4452).

FIG. 42—Tumor and gastrointestinal uptake (healthy organ) PET imaging data in Patient (4) with metastatic PDAC (1 hr after a single 185 MBq dose of Ga⁶⁸-DPI-4452).

FIG. 43—Tumor and gastrointestinal uptake (healthy organ) PET imaging data in Patient (5) with metastatic PDAC (1 hr after a single 185 MBq dose of Ga⁶⁸-DPI-4452).

FIG. 44—Tumor and gastrointestinal uptake (healthy organ) PET imaging data in Patient (6) with metastatic CRC (1 hr after a single 185 MBq dose of Ga⁶⁸-DPI-4452).

### DETAILED DESCRIPTION OF THE PRESENT INVENTION

### 1. Definitions

The expression “compound capable of binding to CAIX” as used herein refers to a peptidic compound, e.g., a compound of formula (1), which can bind CAIX with high affinity. In particular, the compound as used herein (in its unchelated form) can bind CAIX with an affinity (equilibrium dissociation constant (K_{D})) of less than 0.50 nM, preferably less than 0.3 nM. The pharmacological activity of a given compound towards CAIX can be determined by, e.g., the Surface Plasmon Resonance (SPR) assay described in the examples section below.

The compound used in the present invention is DOTA-PPAc-Gln-[Cys(3MeBn)-Glu-pro-Asp-Af3(Cpsu)-Leu-Thr-Trp-Ser-Cys]-NH2, which is represented by the following formula (1):

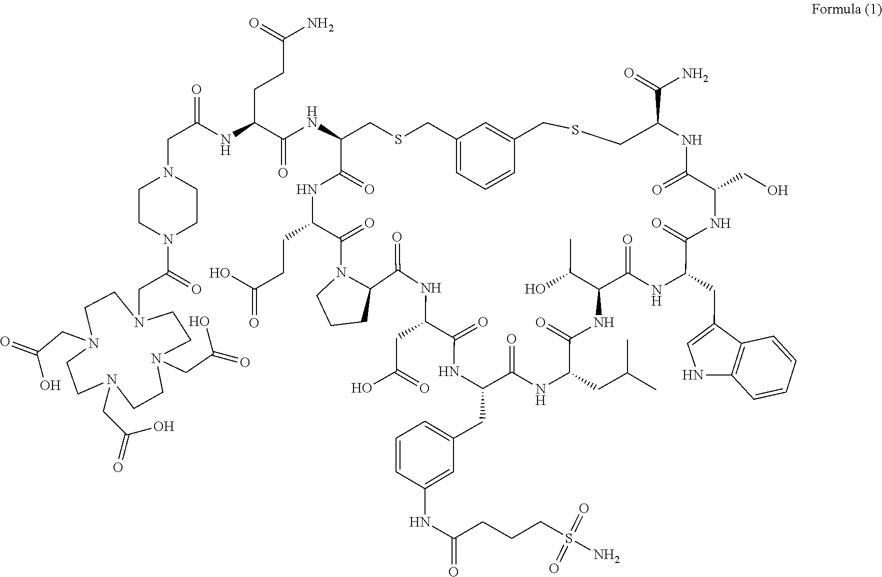 [SMILES: CC(C)C[C@@H]1NC(=O)[C@H](Cc2cccc(NC(=O)CCCS(N)(=O)=O)c2)NC(=O)[C@H](CC(=O)O)NC(=O)[C@H]2CCCN2C(=O)[C@H](CCC(=O)O)NC(=O)[C@@H](NC(=O)[C@H](CCC(N)=O)NC(=O)CN2CCN(C(=O)CN3CCN(CC(=O)O)CCN(CC(=O)O)CCN(CC(=O)O)CC3)CC2)CSCc2cccc(c2)CSC[C@@H](C(N)=O)NC(=O)[C@H](CO)NC(=O)[C@H](Cc2c[nH]c3ccccc23)NC(=O)[C@H]([C@@H](C)O)NC1=O]

The compound of formula (1) chelated with ⁶⁸Ga or ¹⁷⁷Lu (via the DOTA moiety comprised therein) may be herein referred to as ⁶⁸Ga-DPI-4452 or [⁶⁸Ga]-DPI-4452 and ¹⁷⁷Lu-DPI-4452 or [¹⁷⁷Lu]-DPI-4452 respectively. In this context, the term “DOTA moiety” (or “DOTA derivative”) is used to characterize a moiety that differs from 1,4,7,10-tetraazacyclododecane-1,4,7,10-tetraacetic acid (DOTA) only by the structural elements responsible for bonding to adjacent moieties, i.e., by the amide bond formed between a carboxyl group of DOTA and the amino group of the adjacent piperazine moiety.

The compound of Formula (1) is a compound capable of binding to CAIX.

The term “peptide” as used herein refers to a compound comprising a continuous sequence of at least two amino acids linked to each other via peptide linkages, e.g., amide bonds.

The term “amino acid” as used herein refers to a compound that contains or is derived from a compound containing at least one amino group and at least one acidic group, preferably a carboxyl group. The distance between amino group and acidic group is not particularly limited. α-, β-, and γ-amino acids are suitable but α-amino acids and especially α-amino carboxylic acids are particularly preferred. The term “amino acid” encompasses both naturally occurring amino acids such as the naturally occurring proteinogenic amino acids, as well as synthetic (unnatural) amino acids that are not found in nature. In the following, reference to amino acids may be made using the 3-letter amino acid code (Arg, Phe, Ala, Cys, Gly, Gln, etc.) or the 1-letter amino acid code (R, F, A, C, G, Q, etc.). Examples of unnatural amino acids and other building blocks used herein are identified in the table below along with their respective abbreviations.

TABLE 1
Abbreviation, name and structure of unnatural amino-acids and building
blocks

| Abbreviation | Name | Structure |
|---|---|---|
| 3MeBn | 3-Methylbenzylidene | 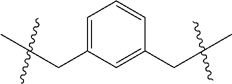 [SMILES: *Cc1cccc(C*)c1] |
| 3MeBnSpa | 3-Metyhlbenzylmercapto- propionic acid | 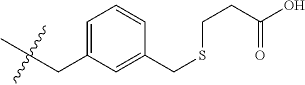 [SMILES: *Cc1cccc(CSCCC(=O)O)c1] |
| Af3 | 3-Amino-phenylalanine | 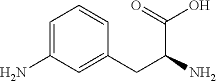 [SMILES: Nc1cccc(C[C@H](N)C(=O)O)c1] |
| PPAc | 4-Carboxymethyl piperazine | 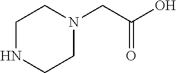 |
| Aph | 4-Aminophenylalanine | 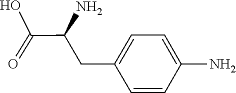 [SMILES: Nc1ccc(C[C@H](N)C(=O)O)cc1] |
| Cpsu | 4-Sulfamoyl-butyric acid | 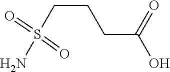 |
| Cys(3MeBn) | | 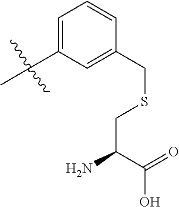 [SMILES: *c1cccc(CSC[C@H](N)C(=O)O)c1] |
| Dap | (S)-2,3-Diaminopropionic acid | 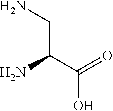 |
| DOTA | 1,4,7,10- Tetraazacyclododecane- 1,4,7, 10-tetraacetic acid | 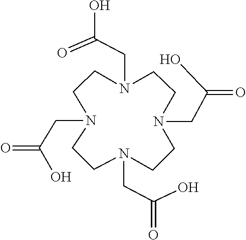 [SMILES: O=C(O)CN1CCN(CC(=O)O)CCN(CC(=O)O)CCN(CC(=O)O)CC1] |
| SaPr | 3-Sulfamoylpropanoic acid | 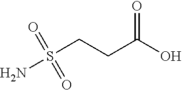 |

Hereinafter, amino acid sequences are written from the N-terminus to the C-terminus (left to right). Unless specified otherwise or dictated otherwise by the context, all connections between adjacent amino acid groups are formed by peptide (amide) bonds.

The term “cancer” as used herein means the pathological condition in mammalian tissues that is characterized by abnormal cell growth to form malignant tumors, which may have the potential to invade or spread to other tissues or parts of the body to form “secondary” tumors known as metastases. A tumor comprises one or more cancer cells.

The term “CAIX positive disease” as used herein refers to diseases that are characterized by expression of CAIX. In some aspects, the term “CAIX positive disease” as used herein refers to cancer. For example, CAIX expression in cancer may be detected by immunohistochemistry (which can detect any level of CAIX expression) or imaging, such as PET or SPECT (which can identify one or more tumor lesions expressing CAIX). In some aspects, the cancer may have a prevalence of CAIX expression of at least 19%, e.g., at least about 20%, at least about 25%, at least about 30%, at least about 40%, at least about 50%, at least about 60%, at least about 70%, at least about 80%. The prevalence of CAIX expression for a particular cancer type can be calculated by measuring the Pathologist H-score in a patient population, as set out in the examples section herein.

In one embodiment, the expression “CAIX positive disease” as used herein refers, specifically, to renal cell carcinoma (RCC), in particular clear cell renal cell carcinoma (ccRCC), colorectal carcinoma (CRC), pancreatic ductal adenocarcinoma (PDAC), glioblastoma (GBM), mesothelioma, cholangiocarcinoma (CCA), ovarian carcinoma, non-small cell lung cancer (NSCLC), in particular squamous non-small cell lung cancer (SNSCLC), brain cancer, pancreatic cancer, thyroid cancer, lung cancer, renal cancer, breast cancer, in particular triple-negative breast cancer (TNBC), head and neck cancer, in particular squamous carcinoma of head and neck (SCCHN), urothelial carcinoma and bladder cancer.

The expression “human patient diagnosed with a CAIX positive disease” as used herein refers to a human patient having a positive diagnosis for a CAIX positive disease, e.g., ccRCC, CRC or PDAC. In some aspects, the patient may also have been positively diagnosed with several of the aforementioned diseases. In one embodiment, a “positive diagnosis” means that the patient has a histological and/or cytological status of disease and, optionally, one or more of the following:
(1) radiographically documented disease progression or recurrence after at least one systemic treatment regimen,
(2) at least one non-irradiated extracranial measurable target lesion as per the Response Evaluation Criteria in Solid Tumors 1.1 (RECIST 1.1) and,
(3) at least one tumor lesion as per Positron Emission Tomography Response Criteria in Solid Tumors 1.0 (PERCIST 1.0).

The expression “tumor uptake” (of radiopharmaceuticals) refers to the biological process in which molecules are taken up by tumor (cancer) cells. Tumor uptake includes tumor cell uptake of molecules (e.g., the compound of formula (1)) and/or the retention thereof in the tumor microenvironment. As a result, the molecules (e.g., the compound of formula (1)) can be present inside the tumor (cancer) cell, at the cell membrane (e.g., accumulated on the cell membrane) and/or within the tumor microenvironment. The accumulation of radioactivity will then damage tumor cell DNA via direct activity by creating DNA single or double strand brakes, or via indirect activity with the generation of free radicals leading to tumor cell death (Desouky et al. Journal of Radiation Research and Applied Sciences 2015, 8(2), 247-254).

The expression “imaging dose” (or “imaging amount”) as used herein refers to the total dose of radioactivity (in Becquerels) administered to a patient to carry out imaging such as PET/CT imaging of the tumor legions/tissues, e.g., in order to diagnose disease progression and/or state.

The expression “treatment dose” (or “treatment amount”) as used herein refers to the total dose of radioactivity (in Becquerels) administered to a patient per cycle in order to treat the CAIX positive disease, e.g., cancer. The treatment dose can be determined by a physician based on dosimetry.

The term “cycle” or “administration cycle” as used herein refers to a period of time starting from when the compound, e.g., labeled with ¹⁷⁷Lu, is administered to the patient and lasting until the next administration. Between administrations the patient is allowed to rest (rest time). The patient may undergo one or more number of cycles, e.g., up to ten cycles. A series of cycles is usually called a “course”, which can last over several months, e.g., 3 to 6 months, depending on the length of each cycle. The length of a cycle and the number of cycles can be determined by a physician based upon the treatment dose, as well as on a variety of factors including the effective dose, the patient's age, body weight, general health, sex, diet, rate of excretion, mode of administration, the type or severity of the disease, and the individual undergoing therapy.

The “effective dose” (or effective amount) as used herein refers to a calculated value, measured in mSv, that takes three factors into account: (1) the absorbed dose to all organs of the body, (2) the relative harm level of the radiation, and (3) the sensitivities of each organ to radiation. The effective dose represents a surrogate of risk, developed for establishing and complying with radiation protection standards.

The term “pharmaceutically acceptable salts” as used herein refers to derivatives of disclosed compounds wherein the parent compound is modified by making acid or base salts thereof. The pharmaceutically acceptable salts include the non-toxic salts or the quaternary ammonium salts of the parent compound formed, for example, from non-toxic inorganic or organic acids or bases. Lists of suitable salts can be found in Remington's Pharmaceutical Sciences, 17^{th} ed., Mack Publishing Company, Easton, PA, 1985, page 1418, S. M. Berge, L. M. Bighley, and D.C. Monkhouse, “Pharmaceutical Salts” J. Pharm. Sci. 1977, 66(1), 1-19; P. H. Stahl and C. G. Wermuth, editors, Handbook of Pharmaceutical Salts: Properties, Selection and Use, Weinheim/Zuirich, Wiley-VCH, 2008 and in A. K. Bansal et al., Pharmaceutical Technology, 3(32), 2008. The pharmaceutical salts can be synthesized from the parent compound which contains a basic or acidic moiety by conventional chemical methods. Unless the context dictates otherwise, all references to compounds of the invention are to be understood also as references to pharmaceutically acceptable salts of the respective compounds.

Where the present description refers to “preferred” embodiments/features, combinations of these “preferred” embodiments/features shall also be deemed as disclosed as long as this combination of “preferred” embodiments/features is technically meaningful.

Hereinafter, in the present description of the invention and the claims, the use of the terms “containing” and “comprising” is to be understood such that additional unmentioned elements may be present in addition to the mentioned elements. However, these terms should also be understood as disclosing, as a more restricted embodiment, the term “consisting of” as well, such that no additional unmentioned elements may be present, as long as this is technically meaningful.

Unless the context dictates otherwise and/or alternative meanings are explicitly provided herein, all terms are intended to have meanings generally accepted in the art, as reflected by IUPAC Gold Book (status of 1 Jun. 2023), or the Dictionary of Chemistry, Oxford, 7^{th} Ed.

Hereinafter, in the present description of the invention and the claims, the use of the term “about” when used in the context of a concentration or an amount is to be understood such that the value is + or −20%.

### 2. Overview

The present invention is based on the finding that the administration to human patients of a treatment dose (or a series of treatment doses in cycles (a course)) of a compound of the invention labeled with ¹⁷⁷Lu may lead to sufficient uptake of the radiolabeled compound in the tumor cells, while unspecific accumulation in healthy tissues (e.g., in the kidneys or other tissues endogenously expressing CAIX) is minimal/below an acceptable level. The invention is further based on the finding that administering to human patients an imaging dose of a compound of the invention labeled with ⁶⁸Ga enables excellent imaging of the tumor tissues, thereby enabling the identification of patients who are likely to benefit from treatment with the compound of the invention labeled with ¹⁷⁷Lu.

One of the most important goals for efficient targeted radionuclide therapy is to achieve a high uptake of the radiolabeled compound, which is largely dependent on the expression level of the targeted protein (CAIX), while reducing side effects due to accumulation in the surrounding tissues and/or organs. The CAIX prevalence studies reported in the experimental section indicate that the compound of the invention labeled with ⁶⁸Ga or ¹⁷⁷Lu, will be effective in the diagnosis and/or treatment of CAIX positive diseases, in particular clear cell renal cell carcinoma (ccRCC), colorectal cancer (RCC) and pancreatic ductal adenocarcinoma (PDAC). The compound of the present invention labeled with ⁶⁸Ga or ¹⁷⁷Lu is thus expected to achieve high uptake in the targeted cells, resulting in excellent imaging (e.g., diagnosis) and/or therapeutic efficacy. Furthermore, the compound of the present invention labeled with ⁶⁸Ga or 177Lu is expected to have excellent biodistribution (i.e., a high tumor-to-healthy tissue ratio) due to the restricted expression profile of CAIX in healthy tissues.

In some aspects, the present inventors have found that the compound of the invention labeled with ⁶⁸Ga or ¹⁷⁷Lu exhibits excellent pharmacokinetic properties, in particular rapid body clearance. When the compound of the invention labeled with ¹⁷⁷Lu is administered over a series of treatments, i.e., over one or more cycles of administration, it is expected that the duration of each cycle can be reduced to less than eight weeks, for example reduced to four weeks. This is particularly advantageous as it is expected that administering the compound labeled with ¹⁷⁷Lu over shorter cycles will provide a more effective treatment of the disease, while maintaining the side effects resulting from the irradiation of the surrounding tissues minimal and/or at an acceptable low level.

### 3. Use of Compound in Methods of Treating Diseases

The compound of the present invention (compound of formula (1)) can be used in methods of treating one or more CAIX positive disease in a human patient. The compound labeled with ¹⁷⁷Lu may be administered to treat, e.g., reduce or stop the progression of the CAIX positive disease(s) via targeted destruction of the tumor cells. In some aspects, the treatment can prolong survival of a patient as compared to expected survival if not receiving the treatment.

The method of treating the CAIX positive disease comprises the optional step of (i) administering to the patient an imaging dose of a compound of formula (1) labeled with ⁶⁸Ga to obtain an image of body parts or tissues to be examined. The body parts or tissues can be visualized by known imaging techniques such as computer tomography techniques such as PET (PET=Positron Emission Tomography); for a review of this technique and its application see e.g. Shankar Vallabhajosula (ed.), Molecular Imaging, Radiopharmaceuticals for PET and SPECT, Springer Verlag or Lucia Martiniova et al., Gallium-68 in Medical Imaging, Current Radiopharmaceuticals, 2016, 9, 187-207. The ⁶⁸Ga-labeled compound can thus be used for diagnosing the progression and/or state of the CAIX positive disease.

In one aspect of the present invention, there is provided the compound of the invention for use in a method of treating a carbonic anhydrase IX (CAIX) positive disease in a human patient, the method comprising the steps of:
(i) optionally administering to the patient an imaging dose of the compound labeled with ⁶⁸Ga, to obtain an image of body parts or tissues to be examined, and
(ii) administering to the patient a treatment dose of the compound labeled with ¹⁷⁷Lu, to treat the CAIX-positive disease,
wherein, preferably the compound labeled with ¹⁷⁷Lu administered in step (ii) is administered once per cycle of one to six weeks, preferably once per cycle of four to six weeks, and most preferably once per cycle of four weeks, and
wherein if the human patient is not administered an imaging dose of the compound labeled with ⁶⁸Ga in step(i) the compound labeled with ¹⁷⁷Lu administered in step (ii) is administered once per cycle of one to six weeks, preferably once per cycle of four to six weeks, and most preferably once per cycle of four weeks.

The compound of Formula (1) has the following formula:

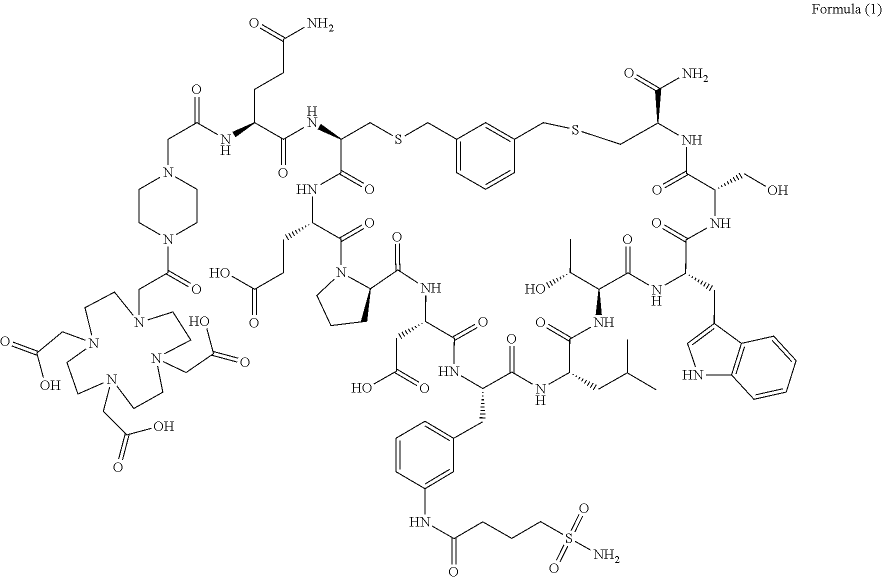 [SMILES: CC(C)C[C@@H]1NC(=O)[C@H](Cc2cccc(NC(=O)CCCS(N)(=O)=O)c2)NC(=O)[C@H](CC(=O)O)NC(=O)[C@H]2CCCN2C(=O)[C@H](CCC(=O)O)NC(=O)[C@@H](NC(=O)[C@H](CCC(N)=O)NC(=O)CN2CCN(C(=O)CN3CCN(CC(=O)O)CCN(CC(=O)O)CCN(CC(=O)O)CC3)CC2)CSCc2cccc(c2)CSC[C@@H](C(N)=O)NC(=O)[C@H](CO)NC(=O)[C@H](Cc2c[nH]c3ccccc23)NC(=O)[C@H]([C@@H](C)O)NC1=O]

wherein the DOTA moiety comprised therein chelates ⁶⁸Ga, or ¹⁷⁷Lu to form the ⁶⁸Ga, or ¹⁷⁷Lu labeled compound.

The biodistribution of the compound of the invention labeled with ⁶⁸Ga (⁶⁸Ga-labeled compound) was found to be very similar to that of the compound of the invention labeled with ¹⁷⁷Lu (¹⁷⁷Lu-labeled compound). Therefore, the imaging with the ⁶⁸Ga-labeled compound may be particularly useful for selecting patients who will benefit from treatment with the compound labeled with ¹⁷⁷Lu. This selection of patients increases the chance of success of any subsequent treatment with the ¹⁷⁷Lu-labeled compound, while at the same time avoiding administration of the ¹⁷⁷Lu-labeled compound in patients that will not benefit from the treatment.

Accordingly, in one embodiment, the method of treating a CAIX positive disease in a human patient, comprises the steps of:
(i) administering to the patient an imaging dose of the compound labeled with ⁶⁸Ga, to obtain an image of body parts or tissues to be examined, and
(ii) administering to the patient a treatment dose of the compound labeled with ¹⁷⁷Lu, to treat the CAIX-positive disease.

It is preferred that the compound labeled with ¹⁷⁷Lu administered in step (ii) is administered once per cycle of one to six weeks, preferably once per cycle of four to six weeks, and most preferably once per cycle of four weeks. Thus, in one embodiment, the method comprises the steps of:
(i) administering to the patient an imaging dose of the compound labeled with ⁶⁸Ga, to obtain an image of body parts or tissues to be examined, and
(ii) administering to the patient a treatment dose of the compound labeled with ¹⁷⁷Lu, to treat the CAIX-positive disease,
wherein the compound labeled with ¹⁷⁷Lu administered in step (ii) is administered once per cycle of one to six weeks, preferably once per cycle of four to six weeks, and most preferably once per cycle of four weeks.

In a specific embodiment, following step (i), patients are selected to proceed to step (ii) if at least 30% e.g., at least 40%, at least 50%, at least 60%, preferably at least 70%, more preferably at least 75%, and even more preferably at least 80% of their cancer lesions (i.e., tumors) express CAIX. In this context, the percentages of lesions/tumors expressing CAIX can be calculated using the following formula:

(Number of positive lesions/tumors detected by PET imaging after administration of a compound of formula (1) labeled with ⁶⁸Ga, divided by the number of all lesions/tumors detected by conventional imaging used in cancer care, such as CT imaging)×100

Primary tumors, as well as metastases, which originate from the primary tumors (e.g., after one or more primary tumors have spread to other parts of the body to form secondary lesions) are included in the calculation.

In step (i) of the method disclosed herein, the method used to obtain an image (visualization) of the body parts or tissues is preferably PET (Positron Emission Tomography). In one embodiment, this method is used to obtain an image of a cancer (associated tumors) selected from clear cell renal cell carcinoma (ccRCC), colorectal cancer (CRC) and pancreatic ductal carcinoma (PDAC).

Visualization is achieved by recording the energy and location of the radiation emitted by ⁶⁸Ga (“tracer”), this information then being used by a computer program to reconstruct three-dimensional (3D) images of tracer concentration within the body. The convenient half-life of ⁶⁸Ga (T½=68 min) provides sufficient radioactivity for various PET imaging applications. ⁶⁸Ga decays 87.94% through positron emission with a maximum energy of 1.9 MeV, mean 0.89 MeV. The ⁶⁸Ga³⁺ cation can form stable complexes with many ligands containing oxygen and nitrogen as donor atoms. This makes ⁶⁸Ga suitable for complexation with chelators and various macromolecules, allowing for kit development.

In modern PET computed tomography scanners, PET images are often reconstructed with the aid of a computed tomography scan performed on the patient during or shortly after the administration of the tracer, in the same device. PET images obtained with ⁶⁸Ga show a very high resolution, typically much higher than that achievable by SPECT (Single Photon Emission Computed Tomography). ⁶⁸Ga can also be used in a diagnostic method using the ⁶⁸Ga-labeled compound of formula (1) as a tracer. SPECT is similar to PET in its use of radioactive tracer material. In contrast to PET, the tracers used in SPECT emit gamma radiation that is measured directly, whereas PET tracers such as ⁶⁸Ga emit positrons that annihilate with electrons up to a few millimeters away, causing two gamma photons to be emitted in opposite directions. A PET scanner detects these emissions “coincident” in time, which provides more radiation event localization information and, thus, higher spatial resolution images than SPECT.

In one embodiment, the CAIX positive disease to be visualized (e.g., diagnosed) and/or treated is cancer, and preferably the human patient has unresectable, locally advanced, or metastatic solid tumors. Preferably, the CAIX positive disease is a cancer selected from the group consisting of renal cell carcinoma (RCC), in particular clear cell renal cell carcinoma (ccRCC), colorectal carcinoma (CRC), pancreatic ductal adenocarcinoma (PDAC), glioblastoma (GBM), mesothelioma, cholangiocarcinoma (CCA), ovarian carcinoma, non-small cell lung cancer (NSCLC), in particular squamous non-small cell lung cancer (SNSCLC), brain cancer, pancreatic cancer, thyroid cancer, lung cancer, renal cancer, breast cancer, in particular triple-negative breast cancer (TNBC), head and neck cancer, in particular squamous carcinoma of head and neck (SCCHN), urothelial carcinoma and bladder cancer.

In a more preferred embodiment, the CAIX positive disease is a cancer selected from the group consisting of ccRCC, CRC, PDAC, SNSCLC, TNBC and SCCHN. Even more preferably, the CAIX positive disease is ccRCC, CRC or PDAC.

In one embodiment, the patient has been diagnosed with a CAIX positive disease before step (i) or (ii), e.g., before the administering of the ⁶⁸Ga-labeled compound, i.e., before step (i). In particular, the patient may have been diagnosed with a CAIX positive disease that is selected from clear cell renal cell carcinoma (ccRCC), colorectal cancer (CRC) and pancreatic ductal carcinoma (PDAC).

The therapeutic effect that is observed after the administration of the ¹⁷⁷Lu-labeled compound can be a reduction in the number of cancer cells, a reduction in tumor size, inhibition or retardation of cancer cell infiltration into peripheral organs, inhibition of tumor growth, and/or relief of one or more of the symptoms associated with the CAIX positive disease.

The imaging dose of the compound of formula (1) labeled with ⁶⁸Ga is from 50 to 250 MBq, preferably from 100 to 200 MBq, more preferably from 150 to 200 MBq, such as about 185 MBq.

The treatment dose of the compound of formula (1) labeled with ¹⁷⁷Lu can range from 0.5 to 25 GBq, such as about 0.66 GBq, or about 1.6 GBq, 1.0 to 25.0 GBq (Giga-becquerels), preferably from 2.0 to 20.0 GBq, more preferably from 3.0 to 18.0 GBq.

A treatment dose can be administered to the patient once every administration cycle e.g., once per cycle of one to six weeks, preferably once per cycle of two to five weeks e.g., 4 weeks. The number of cycles can range from one to a maximum of ten cycles, for instance one to eight or one to six cycles.

In a preferred embodiment, the compound of formula (1) labeled with ¹⁷⁷Lu is administered once per cycle of one to six weeks, preferably once per cycle of four to six weeks, and most preferably once per cycle of four weeks. The administration of the compound over shorter cycles, such as four weeks (28 days), is advantageous as it can provide a more effective treatment of the disease.

In an embodiment, the compound of formula (1) labeled with ¹⁷⁷Lu is administered over one to ten cycles, preferably over two to eight cycles, and more preferably over two to six cycles, in particular over four or six cycles.

In one embodiment, the treatment dose of the compound of formula (1) labeled with ¹⁷⁷Lu is selected from the following doses (1) to (5):
(1) a treatment dose of from 1.5 to 6.0 GBq or 2.0 to 6.0 GBq, such as about 3.7 GBq,
(2) a treatment dose of from 6.0 to 10.0 GBq, such as about 7.4 GBq,
(3) a treatment dose of from 10.0 to 14.0 GBq, such as about 11.1 GBq,
(4) a treatment dose of from 14.0 to 18.0 GBq, such as about 14.8 GBq, and
(5) a treatment dose of from 18.0 to 20.0 GBq, such as about 18.5 GBq.

It is preferred that the treatment dose is selected from (2), (3), (4) and (5).

In one embodiment, the compound is administered to the patient by intravenous injection or infusion, in particular by infusion. In this connection, the compound of formula (1) labeled with ⁶⁸Ga or ¹⁷⁷Lu can be provided as a solution in a pharmaceutically acceptable injectable carrier such as an aqueous carrier (e.g., water or 0.9% sodium chloride). The solution of the compound of formula (1) labeled with with ⁶⁸Ga may have a concentration of from 250 to 950 MBq/8.1 mL, e.g., from 30 to 120 mBq/mL (said concentration may be the concentration as the end of the synthesis of the solution), and the solution of the compound labeled with ¹⁷⁷Lu may have a concentration of from 150 to 900 MBq/mL. The infusion rate can be of from 35 to 60 mL/h, for instance about 50 mL/h.

In one embodiment, the method described herein further comprises the steps of:
(α) preparing an injectable solution of the compound of formula (1) labeled with ⁶⁸Ga or ¹⁷⁷Lu by dissolving the compound in a pharmaceutically acceptable injectable carrier to obtain an infusible solution. In the case of a compound of formula (1) labeled with ⁶⁸Ga, said solution may preferably have a concentration of said compound of 200 to 950 MBq/8.1 mL. In the case of a compound of formula (1) labeled with ¹⁷⁷Lu, said solution may preferably have a concentration of said compound of 150 to 900 MBq/mL; and
(β) administering the injectable solution of the compound obtained from step (α) to the patient, preferably at an infusion rate of 35 to 60 mL/h such as 50 mL/h over an infusion period of 20 to 60 min, e.g., 20 to 35 min. Preferably the infusion will take less than 30 mins.

According to one further embodiment, the compound of formula (1) labeled with ⁶⁸Ga or ¹⁷⁷Lu compound is administered after one or more other therapeutic agents or therapies, such as DNA damage response (DDR) inhibitors, chemotherapeutic agents, immunomodulatory agents, proton pump inhibitors (PPIs), histamine H₂-receptor antagonists, tyrosine kinase inhibitors, cell therapy, external beam radiation, or any other targeted therapies.

### 4. Preparation of the Compound

In the following, methods are provided for preparing the compound of the present invention. The compound can be synthesized relying on standard Fmoc-based solid-phase peptide synthesis (SPPS), including on-resin peptide coupling and convergent strategies. The general strategies and methodology which can be used for preparing and radiolabeling the compound of the present invention are known to the skilled person and also described further below.

### 5. Examples

### 5.1 List of Abbreviations

ACN: acetonitrile
BSA: bovine serum albumin
DCM: dichloromethane
DIEA (or DIPEA): diisopropylethylamine
DMF: N,N-dimethylformamide
DMSO: dimethyl sulfoxide
DTPA: diethylenetriamine pentaacetate
EDT: 1,2-ethanedithiol
EDTA: ethylenediaminetetraacetic acid
EGTA: ethylene glycol-bis(3-aminoethyl ether)-N,N,N′,N′-tetraacetic acid
ESI: electron spray ionization
HATU: 1-[Bis(dimethylamino)methylene]-1H-1,2,3-triazolo[4,5-b]pyridinium 3-oxide hexafluorophosphate
HBTU: 3-[Bis(dimethylamino)methyliumyl]-3H-benzotriazol-1-oxide hexafluorophosphate
HPLC: high-performance liquid chromatography
IU: international unit
LC-MS: high performance liquid chromatography coupled with mass spectrometry
LC/TOF-MS: liquid chromatography time-of-flight mass spectrometry
MTBE: methyl-tert-butylether
PBS: phosphate-buffered saline
QC: quality control
Rt: retention time
RT: room temperature
SQD: single quadrupole detection
SPECT: single-photon emission computed tomography
SPPS: solid-phase peptide synthesis
TBST: tris-buffered saline with Tween 20
TFA: trifluoroacetic acid
TIPS: triisopropylsilane
TIS: triisopropylsilane
Tris: tris(hydroxyethyl)aminomethane
UPLC: ultra-performance liquid chromatography

### 5.2 Materials and Methods

The following materials and methods were used to prepare and evaluate the compound of the present invention.

### 5.2.1 Preparation of the Compounds

Compounds described and used herein (DPI-4452 and DPI-4501) were prepared by standard Fmoc-based SPPS, including on-resin peptide coupling and convergent strategies using an automated peptide synthesizer and a RinkAmide resin in a 50 μmol scale.

Coupling reactions for amide bond formation were performed over 30 min at room temperature using 3 eq of Fmoc-amino-acids activated with HBTU (2.9 eq) in the presence of DIEA (6 eq.). Fmoc deprotection was conducted with a solution of 20% piperidine in DMF. Coupling of the N-terminal labeling moiety can be performed over 30 min at room temperature using 3 eq of DOTA tris-t-Bu ester (Novabiochem) activated with HATU (2.9 eq) in the presence of DIEA (6 eq).
For DPI-4452, the nitro moiety of the Nf3 building block was transformed into an amino function (Af3) as follows: After swelling in DMF, the resin was washed with DMF and then treated with a 1M solution of SnCl₂×2 H₂O in DMF (3 mL per 100 μmol resin, 0.68 g SnCl₂×2 H₂O in 3 mL DMF) overnight. Afterward the resin was washed thoroughly with DMF. The resulting amino function was acylated by addition of 3-carboxypropanesulfonamide (41.8 mg, 0.25 mmol, 5 eq.), HATU (95.1 mg, 0.25 mmol, 5 eq.) and DIPEA (85.6 μl, 0.5 mmol, 10 eq.) in 1.5 mL DMF. The reaction was allowed to proceed under gentle agitation at RT overnight.
After the completion of the assembly of the sequence the resin was finally washed with DCM (3 ml, 4×1 minute), dried in the vacuum overnight and treated with TFA, EDT, water and TIPS (94/2.5/2.5/1) for 2 h (unless otherwise stated). Afterwards the cleavage solution was poured into a chilled mixture of MTBE and cyclohexane (1/1, 10-fold excess compared to the volume of cleavage solution), centrifuged at 4° C. for 5 minutes and the precipitate collected and dried in the vacuum. The residue was lyophilized from water/acetonitrile prior to purification or further modification.
The crude peptide material was dissolved in a 1:1 mixture of ammonium bicarbonate solution (50 mM, pH=8.5) and acetonitrile. To the resulting mixture a solution of α,α′-dibromo-m-xylene in acetonitrile was added. Upon completition of the cyclization reaction which was judged by analytical LC-MS, TFA was added and the reaction solution subjected to lyophilization. The volume of solvent, amount of α,α′-dibromo-m-xylene and volume of TFA used in the reaction depended on the amount of resin used for the synthesis of the linear peptide precursor—per 50 pmol of initially used 60 mL of the solvent mixture, 14.5 mg (55 μmol) of α,α′-dibromo-m-xylene and 50 μL of TFA were used.
The remainder obtained after lyophilization was purified by preparative HPLC (15 to 35% B in 20 min—Kinetex) to yield 14.00 mg of the pure DPI-4452 (13.3%). HPLC: Rt=5.69 min. LC/TOF-MS: exact mass 2104.9006 (calculated 2104.8693). C92H132N22029S3 (MW=2106.364).
For DPI-4501, the nitro moiety of the Nif building block was transformed into an amino function (Aph) as follows: After swelling in DMF, the resin was washed with DMF and then treated with a 1M solution of SnCl₂×2 H₂O in DMF (3 mL per 100 μmol resin, 0.68 g SnCl₂×2 H₂O in 3 mL DMF) overnight. Afterward the resin was washed thoroughly with DMF. The resulting amino function was acylated by addition of 3-sulfamoylpropanoic acid (38.3 mg, 0.25 mmol, 5 eq.), HATU (95.1 mg, 0.25 mmol, 5 eq.) and DIPEA (85.6 μl, 0.5 mmol, 10 eq.) in 1.5 mL DMF. The reaction was allowed to proceed under gentle agitation for 5 h at RT.
After the completion of the assembly of the sequence the resin was finally washed with DCM (3 ml, 4×1 minute), dried in the vacuum overnight and treated with TFA, EDT, water and TIPS (94/2.5/2.5/1) for 2 h (unless otherwise stated). Afterwards the cleavage solution was poured into a chilled mixture of MTBE and cyclohexane (1/1, 10-fold excess compared to the volume of cleavage solution), centrifuged at 4° C. for 5 minutes and the precipitate collected and dried in the vacuum. The residue was lyophilized from water/acetonitrile prior to purification or further modification.
The crude peptide material was dissolved in a 1:1 mixture of ammonium bicarbonate solution (50 mM, pH=8.5) and acetonitrile. To the resulting mixture a solution of α,α′-dibromo-m-xylene in acetonitrile was added. Upon completition of the cyclization reaction which was judged by analytical LC-MS, TFA was added and the reaction solution subjected to lyophilization. The volume of solvent, amount of α,α′-dibromo-m-xylene and volume of TFA used in the reaction depended on the amount of resin used for the synthesis of the linear peptide precursor—per 50 μmol of initially used 60 mL of the solvent mixture, 14.5 mg (55 μmol) of α,α′-dibromo-m-xylene and 50 μL of TFA were used.
The remainder obtained after lyophilization was purified by preparative HPLC (15 to 40% B in 20 min—Kinetex) to yield 9.95 mg of the pure DPI-4501 (10.12%). HPLC: Rt=5.633 min. LC/TOF-MS: exact mass 1964.7734 (calculated 1964.7743). C85H120N20028S3 (MW=1966.181).

### 5.2.2 Radiolabeling of the Compounds

### Gallium Radiolabeling:

To prepare the Gallium-labelled compound (⁶⁸Ga-DPI-4452), an eluate of a gallium generator was added to a solution of DPI-4452 60 μg (prepared as described above), ascorbic acid 2.2 mg and ethanol (519 μL). A solution of sodium acetate trihydrate (80 mg) (pH 3.9) was added and the reaction mixture was heating at 90° C. for 15 min. Prior to QC sampling, the solution was allowed to cool down for 10 min. The radiochemical purity was analyzed by thin layer chromatography (TLC) 5 μL sample on silica gel plate. Mobile phase 77 g/L solution of ammonium acetate in water, methanol 50:50 VN. Detection with a detector suitable to determine the distribution of radioactivity. No more than 3% of free gallium-68 was detected.

### Lutetium Radiolabeling:

To prepare the lutetium-labeled compound (¹⁷⁷Lu-DPI-4452), a solution of DPI-4452 prepared as described above and ¹⁷⁷Lu (available from ITM or Isotopia) was prepared in a 0.58 M ascorbic acid (+2M NaOH) reaction buffer (pH 4.5) and the labeling was carried out at 80° C. for 15 min. The lutetium incorporation was analyzed by standard HPLC using a C18 column and reached above 95% efficiency.

### Indium Radiolabeling:

To prepare the indium-labeled compound (¹¹¹In-DPI-4452), a solution of DPI-4452 was added to the radionuclide solution (¹¹¹InCl₃ in 20 mM HCl, available from Curium). Labeling buffer (sodium acetate pH 5.3) was added to a final concentration of 0.1 M buffer. After heating for 25 min at 80° C., the reaction mixture was allowed to cool down for 5 min before adding 1 μl 10 mM DTPA and 1 μl 5% TWEEN-20 per 50 μl. For quality control, the reaction mixture was diluted 1:10 in HPLC sample diluent (0.1% TWEEN-20 in 0.1 M sodium acetate pH 5.3).
Labeling efficiency and radiochemical purity were determined by HPLC using an Agilent Poroshell HPH C18 column (gradient: 5% acetonitrile (ACN) to 70% ACN in 0.1% TFA in water within 15 min; flow rate: 0.5 ml/min). Labeling efficiency and radiochemical purity of ¹¹¹In-DPI-4452 was greater than 94%.

### Example 1: Prevalence of CAIX Expression in CRC, PDAC, Sq. NSCLC, SCCHN, TNBC and ccRCC Cancers

CAIX protein expression was assessed using a validated immunohistochemistry assay (IHC) with an anti-CAIX antibody (M75) on a panel of 30 ccRCC, 70 PDAC, 80 Sq. NSCLC, 60 SCCHN, 95 TNBC and 85 CRC tumor specimens as well as healthy tissue. H-score was calculated for each individual sample.

The IHC method was adapted from Rasheed S. et al. (see Pathol Res Pract. 2009, 205(1), 1-9). Tissue Microarrays (TMAs) containing colon carcinoma specimens (#BC000110), healthy normal colon tissue (#C0727), normal lung tissue (#LCN241), lung SCC (#LC808b), mixed pancreatic tissues (#PA482, #PA805c), a breast cancer (#BR1901), a head and neck cancer (#HN601d), a normal multi-organ (#FDA999w), ccRCC specimens and non-tumoral adjacent kidney tissue (#KD601a) panel were purchased from US Biomax and used for validation.

The CA9 (mouse clone M75) assay was evaluated on a semi-quantitative scale, and the percentage of tumor cells or normal cells staining at each of the following four levels was recorded: 0 (no staining), 1+ (weak staining), 2+ (moderate staining) and 3+ (strong staining). A tumor or normal sample was considered positive if at least 1% of cells demonstrated positive expression. The subcellular localization (SCL) of staining was noted for positive samples.

### Pathologist Tumor H-Score

The Pathologist H-Score was calculated based on the summation of the product of percent of cells stained at each staining intensity using the following equation: (3×% cells staining at 3+)+ (2×% cells staining at 2+)+ (1×% cells staining at 1+).

The measured CAIX prevalence is shown in the table below with respect to each tumor type.

TABLE 2
Prevalence of CAIX expression in CRC, Sq.
NSCLC, PDAC, SCCHN, TNBC and ccRCC cancers

| | H-Score | | | Sq. | | | |
|---|---|---|---|---|---|---|---|
| Tissue | (max = 300) | CRC | ccRCC | NSCLC | PDAC | SCCHN | TNBC |
| Malignant | >150 | 29% | 83% | 19% | 40% | 23% | 19% |
| | | (25/85) | (25/30) | (15/79) | (26/65) | (14/60) | (10/54) |
| | >100 | 35% | 83% | 20% | 51% | 33% | 24% |
| | | (30/85) | (25/30) | (16/79) | (33/65) | (20/60) | (10/54) |
| | >40 | 56% | 87% | 42% | 60% | 62% | 37% |
| | | (48/85) | (26/30) | (33/79) | (39/65) | (37/60) | (10/54) |
| Healthy | >40 | 0% | 0% | 0% | 0% | n.d. | n.d. |
| control | | (0/21) | (0/30) | (0/22) | (0/30) | | |

### Example 2: In Vitro Binding Assay of DPI-4452, ^{nat}Lu-DPI-4452 and ^{nat}Ga-DPI-4452 to CAIX

The affinity and the kinetics of DPI-4452 binding to CAIX was evaluated in a cell-free assay using Surface Plasmon Resonance (SPR) approach. Human Fc-recombinant protein was captured on the sensor chip and DPI-4452 or ^{nat}Lu-DPI-4452 or ^{nat}Ga-DPI-4452 at different concentration were injected into the system and the association and dissociation of the molecules to the target were determined (Table 3).

TABLE 3
Affinity and kinetic binding properties
of DPI-4452 for human CAIX

| Compound | K_{D} (nM) * | t_{1/2} (min) |
|---|---|---|
| DPI-4452 | 0.25 | 99 |
| ^{nat}Lu-DPI-4452 | 0.16 | 123 |
| ^{nat}Ga-DPI-4452 | 0.2 | 112 |

* K_{D} = equilibrium dissociation constant,
min = minutes,
t_{1/2} = half-life

DPI-4452, ^{nat}Lu-DPI-4452 and ^{nat}Ga-DPI-4452 compounds bind to CAIX with subnanomolar affinity and show slow dissociation kinetics. The mean dissociation half-life of the test compounds was 99 min for DPI-4452, 123 min for ^{nat}Lu-DPI-4452 and 112 min for ^{nat}Ga-DPI-4452. These results demonstrated that labeling of DPI-4452 with lutetium or gallium does not impact its affinity and kinetic properties for CAIX binding.

### Materials and Methods

Surface Plasmon Resonance (SPR) studies were performed on a Biacore™ T200 using a ligand capture approach. Basically, anti-human Fc antibodies were immobilized on the surface of a sensor chip and CAIX was captured on the functionalized surface via its Fc Tag. Then, each compound was injected at increasing concentrations on the captured CAIX in order to measure the in real-time interaction of the compound to its target (i.e., the captured CAIX). The in real-time monitoring of the association and dissociation of the interaction gives access to the interaction kinetics parameters (i.e., the association and dissociation rate constants and the resulting affinity constants).

For each interaction assay, the background was measured on a reference flow-cell with no captured CAIX and was subtracted to the signal measured on the active flow-cell surface. Moreover, the baseline drift was corrected by performing an entire interaction cycle with the injection of running buffer instead of the compound on the active flow-cell surface (double referencing).

### Immobilization of the Human Fc-Capture Antibody Via Amine Coupling:

A series S CM5 sensor chip was used (Cytiva).
All reagents used for the immobilization procedure were part of the Amine Coupling Kit, type 2 and/or the Human Antibody Capture Kit (Cytiva). The running buffer (HBS-EP+) was prepared by 10-fold dilution of the stock HBS-EP+ 10× solution (Cytiva).
Anti-human Fc antibodies stock solution (0.5 mg/mL in 0.15 M NaCl) was diluted in 10 mM sodium acetate pH 5.0 to a final concentration of 25 μg/mL directly into a plastic vial 7 mm.
Amine coupling reagents were transferred into plastic vials 7 mm as follow:
   95 μL of 0.4 M 1-ethyl-3-(3-dimethylaminopropyl)carbodiimide (EDC).
   95 μL of 0.1 M N-hydroxysuccinimide (NHS).
   An empty plastic vial 7 mm was added in the Biacore™ sample rack for mixing.
   150 μL of 1 M ethanolamine-HCl, pH 8.5.
Plastic vials were closed with rubber caps and the sample rack was inserted into the Biacore™ T200 sample compartment.
The Biacore™ T200 Amine Wizard was used to immobilize the anti-Human Fc antibodies on the four flow-cells of a series S CM5 sensor chip simultaneously with defined contact time of 360 seconds and flow-rate of 5 μL/min.
The immobilization procedure was performed at 25° C., following Cytiva's recommendations, as described in Table 4.

TABLE 4
Immobilization procedure of the anti-human Fc antibodies
on the series S CM5 sensorchip surface

| | | Contact | Flow-rate | Injected |
|---|---|---|---|---|
| Step | Injected solution | time (s) | (μL/min) | Volume |
| Surface activation | EDC/NHS | 420 | 10 | 70 μL |
| Ligand immobilization | Anti-human Fc | 360 | 5 | 30 μL |
| | antibodies at 25 | | | |
| | μg/mL | | | |
| Surface deactivation | 1M ethanolamine | 420 | 10 | 70 μL |

This procedure allowed to reach immobilization levels between 6000 and 9000 RU of anti-human Fc antibodies on the sensor chip surface.

### Preparation of the Human CAIX Solution:

CAIX (Sino Biological, 10107-H02H) stock solution was prepared from lyophilized material reconstituted with deionized water to a concentration of 0.25 μg/mL (3.69 μM), following the supplier recommendations.
CAIX working solution was obtained after dilution of CAIX stock solution with PBS-P+ 1×, 0.1% DMSO to a final concentration of 200 nM directly in a Biacore™ plastic vial.

### Preparation of the Compound Dilutions:

Preparation of intermediate 1 mM compound solutions:
   A stock solution was prepared by dissolving each compound in a respective volume of 0.1 M sodium acetate, pH 5.0 to obtain a final concentration of 1 mg/mL (474.74 μM for DPI-4452, 438.92 μM for ^{nat}Lu-DPI-4452 and 460.18 μM for ^{nat}Ga-DPI-4452).
   1 mL of intermediate 1 mM compounds solutions were prepared as follows:
      DPI-4452: 2.1 μL of stock solution was diluted in 997.9 μL of PBS-P+ 1×, 0.1% DMSO.
      ^{nat}Lu-DPI-4452: 2.3 μL of stock solution was diluted in 997.7 μL of PBS-P+ 1×, 0.1% DMSO.
      ^{nat}Ga-DPI-4452: 2.2 μL of stock solution was diluted in 997.8 μL of PBS-P+ 1×, 0.1% DMSO.
Preparation of the compounds working solutions:
   For each compound, working solutions were prepared by serial dilutions directly in the 96-well microplate as follows:
      The 50 nM working solution was prepared by dilution of 10 μL of the 1 mM intermediate dilution in 190 μL of PBS-P+ 1×, 0.1% DMSO.
      The 12.5 nM working solution was prepared by dilution of 50 μL of the 50 nM intermediate dilution in 150 μL of PBS-P+ 1×, 0.1% DMSO.
      The 3.125 nM working solution was prepared by dilution of 50 μL of the 12.5 nM intermediate dilution in 150 μL of PBS-P+ 1×, 0.1% DMSO.
      The 0.78 nM working solution was prepared by dilution of 50 μL of the 3.125 nM intermediate dilution in 150 μL of PBS-P+ 1×, 0.1% DMSO.
      The 0.19 nM working solution was prepared by dilution of 50 μL of the 0.78 nM intermediate dilution in 150 μL of PBS-P+ 1×, 0.1% DMSO.

### Kinetics and Affinity Constants Determination:

All closed plastic vials and the sealed 96-well microplate were placed into the sample rack and inserted into the Biacore™ T200 sample compartment.
The Biacore™ fluidic system was primed with PBS-P+ 1×, 0.1% DMSO and three startup cycles were performed to ensure the correct conditioning of the system before measurements.
Three different CAIX capture levels were evaluated of flow-cell 2 (~1640 RU), flow-cell 3 (~1150 RU) and flow-cell 4 (~670 RU) in parallel.
A reference flow-cell (flow-cell 1) was used on which anti-human Fc antibodies were immobilized with the same density than on the active flow-cells (flow-cells 2, 3 and 4) but without the CAIX capture step.
Kinetics measurements were performed in Single Cycle Kinetics (SCK) mode at 25° C., in triplicates.
Before each measurement cycle, a blank cycle was performed by injecting running buffer (PBS-P+ 1×, 0.1% DMSO) instead of the compound solution through the active flow-cells.
3 M magnesium chloride (Cytiva, part of the human antibody capture kit) was injected at the end of each cycle in order to regenerate the surface.
The binding kinetics measurements procedure is detailed in Table 5.

TABLE 5
Binding kinetics measurements procedure.

| | | | Flow- | |
|---|---|---|---|---|
| | | Contact | rate | |
| Step | Injected solution | time (s) | (μL/min) | Flow-cell |
| Capture 1 | 200 nM CAIX | 200 | 5 | 2 |
| Capture 2 | 200 nM CAIX | 100 | 5 | 3 |
| Capture 3 | 200 nM CAIX | 50 | 5 | 4 |
| Compound concentration 1 | 0.19 nM dilution of compound | 120 | 30 | 1, 2, 3 and 4 |
| Compound concentration 2 | 0.78 nM dilution of compound | 120 | 30 | 1, 2, 3 and 4 |
| Compound concentration 3 | 3.125 nM dilution of compound | 120 | 30 | 1, 2, 3 and 4 |
| Compound concentration 4 | 12.5 nM dilution of compound | 120 | 30 | 1, 2, 3 and 4 |
| Compound concentration 5 | 50 nM dilution of compound | 120 | 30 | 1, 2, 3 and 4 |
| Dissociation phase | PBS-P + 1X, 0.1% DMSO | 1200 | 30 | 1, 2, 3 and 4 |
| Regeneration | 3M magnesium chloride | 120 | 20 | 1, 2, 3 and 4 |
| Stabilization phase | PBS-P + 1X, 0.1% DMSO | 120 | 20 | 1, 2, 3 and 4 |

### Data Processing and Statistical Analysis:

For each measurement, the raw surface plasmon resonance response (RU) was plotted against time (s) as a sensorgram using the Biacore™ T200 Control Software.
Raw data were corrected by double referencing with subtraction of the signal of both the reference flow-cell and the blank cycle.
The association and dissociation rate constants (kₐ and k_{d}, respectively), as well as the dissociation constant K_{D} and dissociation half-life t½ were determined from the corrected sensorgrams by fitting the data to a 1:1 Langmuir binding model with the Biacore™ T200 evaluation software.

### Example 3: In Vivo Efficacy of ¹⁷⁷Lu-DPI-4452 in HT-29 (CRC) and SK-RC-52 (ccRCC) Human Cancer Cell Line Xenograft Mouse Models

The human colorectal cancer cell line HT-29 was cultured in Modified McCoy's 5a Medium supplemented with 10% FBS+1% Pen/Strep, and the human clear cell renal cancer cell line SK-RC-52 was cultured in RPMI-1640 GlutaMax-I supplemented with 10% FBS+1% Pen/Strep.

2×10⁶ cells were suspended in 100 μL PBS and Matrigel (1:1) and subcutaneously implanted into the neck of anesthetized female immunodeficient NMRI nude mice. Tumor volume (0.52×(length×width²)) and animal weight was monitored twice weekly until 42 days post treatment initiation. Animals were humanely euthanized by cervical dislocation at predefined study or humane endpoints.

Animals were randomized into equal groups based on tumor volume and body weight. Treatments were initiated at a mean group tumor volume of 140-180 mm³ and administered intravenously in the tail vein in a 100 μL dosing volume.

For both models, treatment groups consisted of 10 mice per group and received either a A) Single administration (day 1) of vehicle, B) Single administration (day 1) of 100 MBq of ¹⁷⁷Lu-DPI-4452, C) Single administration (day 1) of 33 MBq of ¹⁷⁷Lu-DPI-4452 or D) Three administrations (day 1, 8, 15) of 33 MBq of ¹⁷⁷Lu-DPI-4452. To correlate the ⁶⁸Ga-DPI-4452 signal with the ¹⁷⁷Lu-DPI-4452 uptake in tumors, an additional satellite group E of 6 mice received a single administration (day 1) of 10 MBq of ⁶⁸Ga-DPI-4452 followed by a single administration (day 8) of 33 MBq of ¹⁷⁷Lu-DPI-4452.

In groups A-D, radioactivity uptake (as % of injected dose/gram tissue) was assessed in the tumor, kidney, and liver in 3 animals per treatment group by whole-body SPECT/CT imaging (nanoScan SPECT/CT, Mediso) at 4 h after each ¹⁷⁷Lu-DPI-4452 administration, using the following parameters:
Imaging bed: Two/three mice bed
CT acquisition
   Helical scan, 480 projections
   Pitch=1.0
   Voltage=50 kVp
   Exposure=170 ms
SPECT acquisition:
   Pinhole SPECT
   Energy windows: Primar Peak 208 full width: 20%, Secondary 112.9, Tertiary: 56.1 keV
   Acquisition time: Up to 30 min
   Frame time: 30 s

In the satellite mice (group E), radioactivity uptake (as % of injected dose/gram tissue) was assessed in the tumor, kidney, and liver in all 6 animals by both whole-body SPECT imaging at 4 h after administration of 33 MBq ¹⁷⁷Lu-DPI-4452, and by whole-body PET/CT imaging (nanoScan PET/CT, Mediso) at 1 h after 10 MBq ⁶⁸Ga-DPI-4452 administration, using the following parameters:
Animals per bed: 3
PET acquisition time: Up to 10 min
PET acquisition start: 1 hour after injection

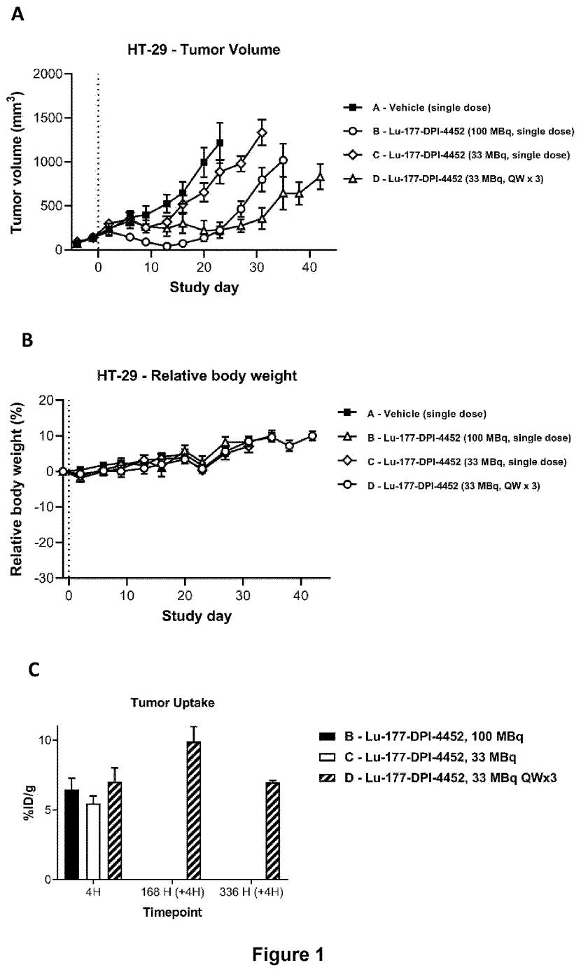

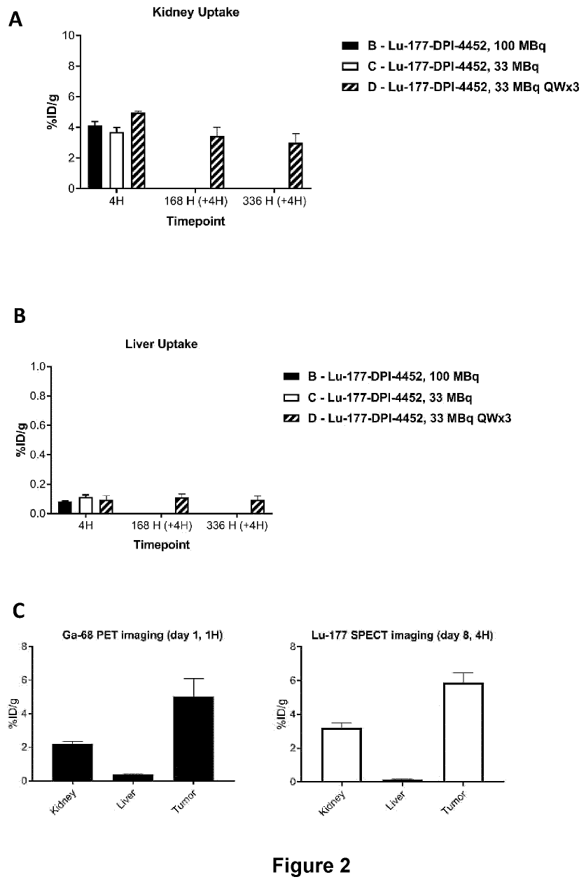

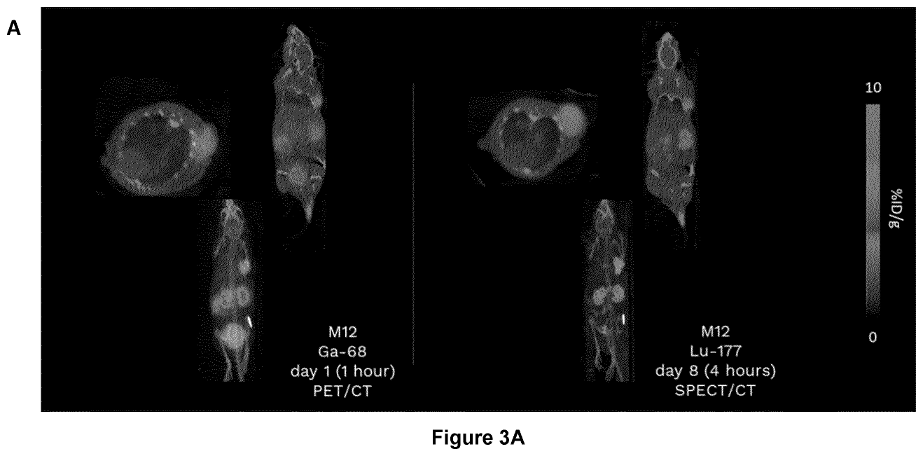

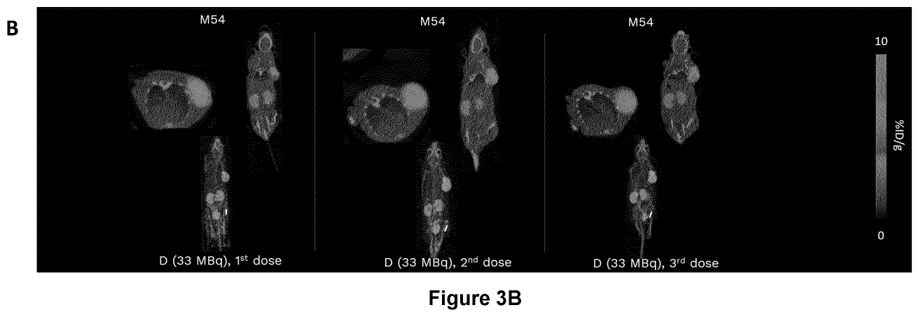

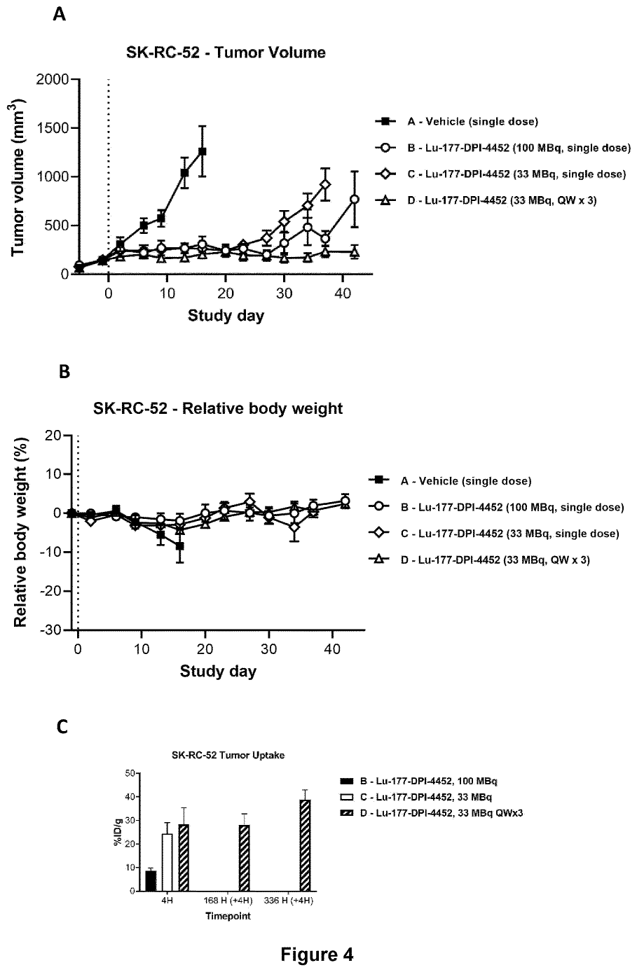

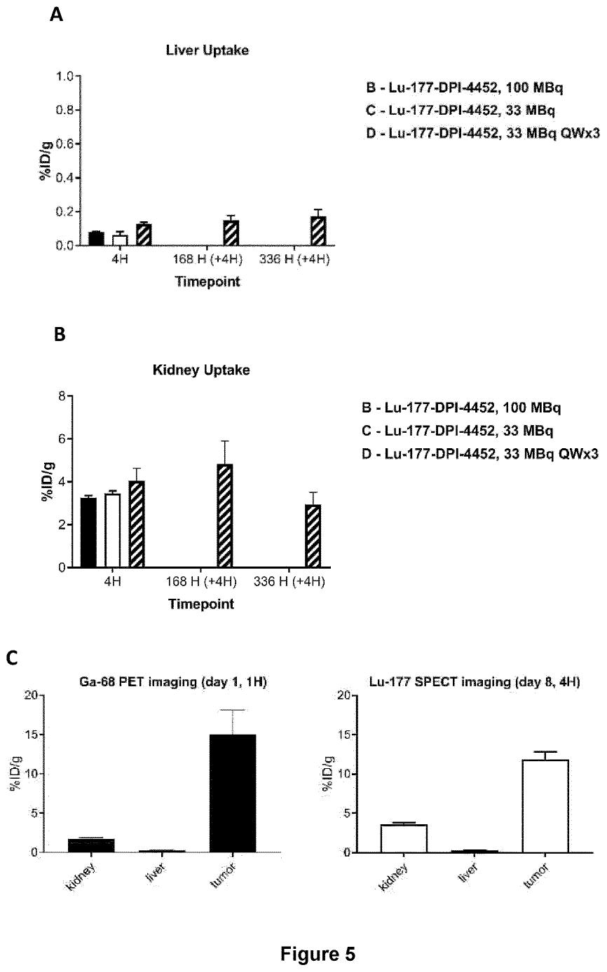

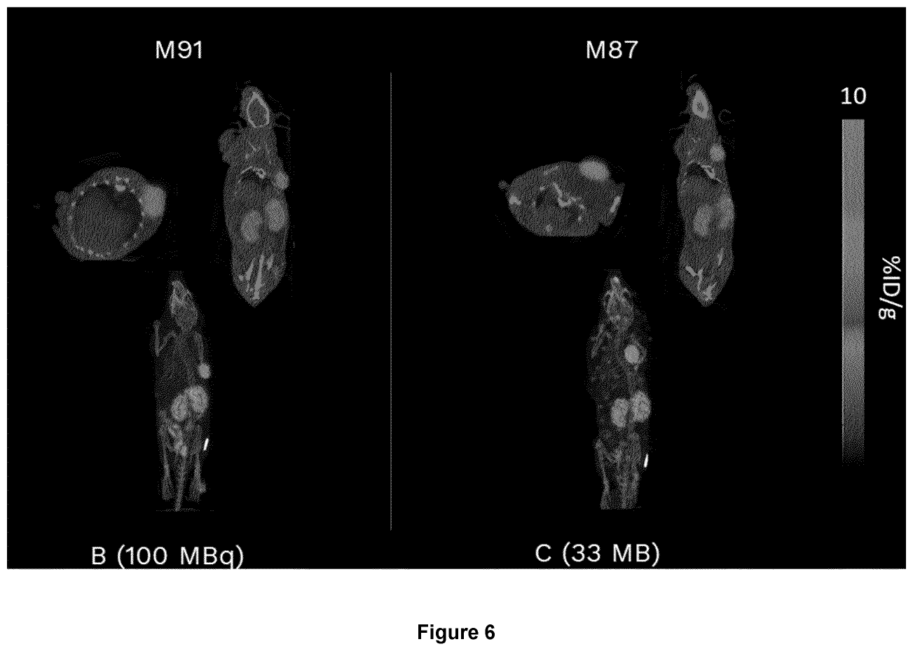

One week later the 6 satellite mice were then assessed again for radioactivity uptake by whole-body SPECT/CT imaging at 4 h after the single ¹⁷⁷Lu-DPI-4452 administration using parameters as described above. The results are shown in FIGS. 1 to 6.

For both models blood sampling for hematology was performed on all animals in group A-D at study day−1, 7, 14 and at study end. Each mouse was restrained and 200 μl blood was obtained from the sublingual or jugular vein in EDTA-tubes and analyzed on the sampling day on a ProCyte Dx Hematology Analyzer with mouse settings. In addition, for the SK-RC-52 model at study day 14 and at study end (day 43), excess blood from the hematology analysis was spun and plasma was obtained (centrifugation at 2000 g for 10 mins at 4° C. in EDTA tubes). The plasma was analyzed for creatinine and urea concentrations using a KONELAB PRIME 30i instrument (Thermo Fisher Scientific).

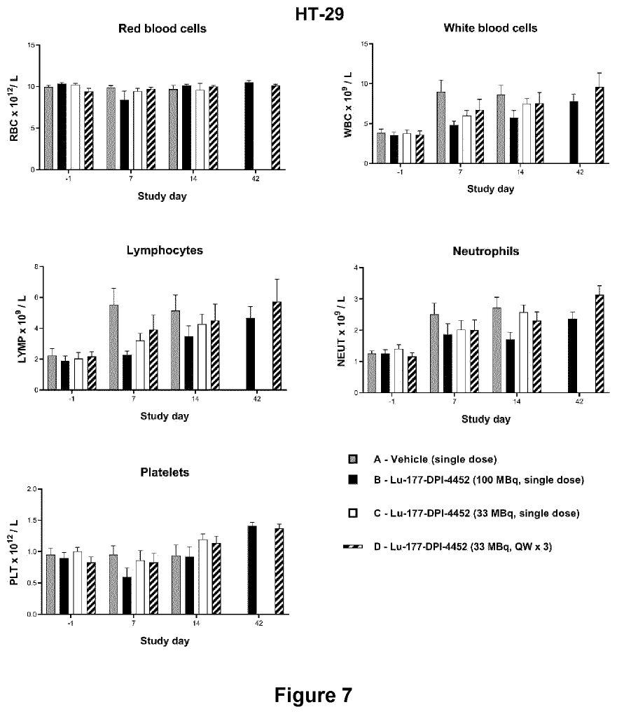

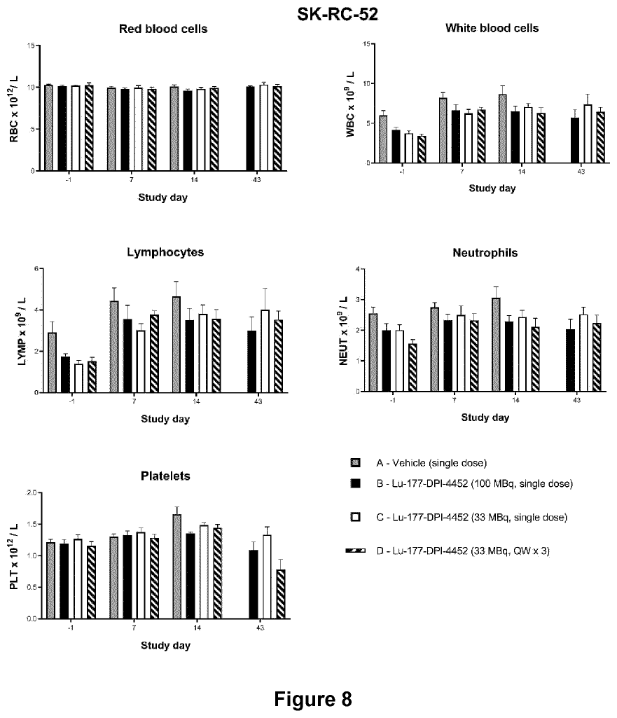

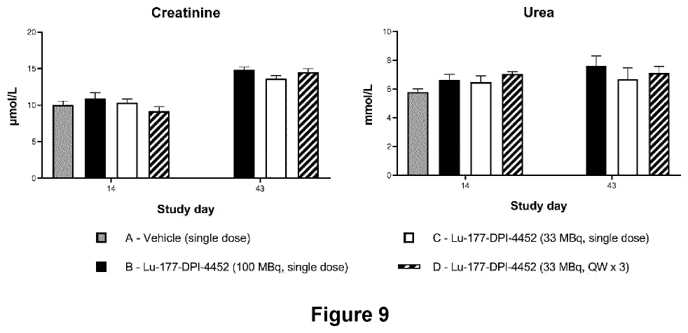

All treatments with ¹⁷⁷Lu-DPI-4452 or ⁶⁸Ga-DPI-4452 were well tolerated in both models. White blood cells, lymphocytes and neutrophils showed increased levels after treatment initiation in all groups for both tumor models when compared to the baseline. Animals dosed with ¹⁷⁷Lu-DPI-4452 (33/100 MBq) showed a relative suppression when compared to animals dosed with vehicle for both tumor models. No differences in creatinine and urea levels between treatment groups were observed at any timepoint. Creatinine values were slightly elevated at study end for the treatment groups, but no control animals were alive at this point for comparison. Results are shown in FIGS. 7 to 9.

SPECT/CT imaging of HT-29 and SK-RC-52 xenografted animals at 4 h after injection of ¹⁷⁷Lu-DPI-4452 demonstrated rapid and high tumor uptake in both models. Tumor uptake remained stable over multiple ¹⁷⁷Lu-DPI-4452 weekly injections in the groups that received three weekly doses of 33 MBq (group D; QW×3). Preferential uptake in the tumor compared to kidney and liver was observed for all groups in both models. A strong and dose-dependent tumor growth inhibition (maximal T/C<20%) was observed in the 100 MBq and the 3×33 MBq treatment groups for the HT-29 model and the 100 MBq, 3×33 MBq, and the 33 MBq treatment groups for the SK-RC-52 model. In both models the dose fractionation (3×33 MBq) seemed beneficial in the long run, and in the SK-RC-52 model led to tumor stasis sustained until study end (day 42). ⁶⁸Ga-DPI-4452 tumor, kidney and liver uptake evaluated by PET imaging closely matched the ¹⁷⁷Lu-DPI-4452 tumor uptake evaluated by SPECT imaging obtained 1 week later for the same animals. Preferential tumor uptake was observed in both models.

### Example 4: Biodistribution Study in Tumor-Bearing Mice with or without Kidney Protection

The biodistribution of [¹¹¹In]In-DPI-4452 and [¹¹¹In]In-DPI-4501 was evaluated in the SK-RC-52 and HT-29 tumor models established subcutaneously in NMRI nude mice, as well as in kidneys, liver and heart (estimate of blood). The effect on tumor uptake of prior injection of gelofusine for kidney protection was investigated in mice bearing the SK-RC-52 tumor. Biodistribution was evaluated using in vivo SPECT/CT imaging. Prior to the in vivo study the compounds (ligands) were initially radiolabeled with In-111 and the stability of radiolabeled compounds was evaluated in buffer at three different timepoints.

The study was performed in female NMRI nude mice (approximately 6 weeks of age) from Janvier Labs, France, housed up to 5 mice per cage.

Indium In-111 chloride (370 MBq/mL at activity reference time) was obtained from Curium and stored at room temperature before use. DPI-4452 and DPI-4501 were stored at −20° C. before use.

DPI-4452 (0.42 mg/mL (199 nmol/mL) solution in 0.1 M HEPES, pH 7) and DPI-4501 (0.40 mg/mL (203 nmol/mL) solution in 0.1 M HEPES, pH 7) were labeled at a molar activity of 30 MBq/nmol peptide according to the following procedure:
In-111 (in 0.02 M HCl) and ligand (i.e., either DPI-4452 or DPI-4501) were mixed, and 1 M sodium acetate, pH 5.5 was added (10% of the final volume).
The reaction mixture was stirred for 25 min at 80° C. at 600 RPM, and then cooled to room temperature and quenched with 5 μL of 50 μM DTP.
The mixture was then diluted with water to a final volume of 10 mL and transferred onto the purification column (previously conditioned with 5 mL 99.9% EtOH followed by 5 mL water). The column was washed with 2 mL water, and then the final product was eluted from the column in EtOH solution (50%)
The sample was formulated in PBS to the final concentration of 150 MBq/mL and 5 nmol/mL.

Quality control was conducted using one HPLC method and one TLC method. The HPLC QC method employed a Thermo Scientific Vanquish HPLC system including a UV detector set at 220 nm, a GABI Nova radiodetector and an XBridge C18 3.5 μm 4.6×50 mm column. Chromatography was conducted at room temperature at a flow rate of 1.5 mL/min using a mobile phase consisting of A: 0.1% trifluoroacetic acid in water; and B: 0.1% trifluoroacetic acid in acetonitrile, according to the following linear gradient: 0-7 min from 5% B to 95% B; 7-8.5 min from 95% B to 5% B, 8.5-11 min: 5% B. The TLC QC method employed 11-cm long plates with an iTLC-SG stationary phase; the mobile phase was 0.1 M citric acid, pH 5.4; the sample volume was 2 μL; the detector was a miniGita OFA Probe. The release criteria for the labeled compounds at EOS (end-of-synthesis) were 90% radiochemical purity from both HPLC and TLC methods.

SK-RC-52 is a human renal cell carcinoma cell line derived from one metastatic site in mediastinum of a 61-year-old female patient. HT-29 is a human colorectal adenocarcinoma cell line derived from the primary tumor of a 44-year-old female patient.

SK-RC-52 cells were cultured in RPMI 1640 with GlutaMax-I (Thermo Fisher Scientific #61870044) supplemented with 10% fetal bovine serum+1% penicillin/streptomycin, harvested, washed twice in RPMI 1640 and resuspended at 2×10⁷ cells/mL in RPMI 1640. HT-29 cells were cultured in McCoy's 5a Medium Modified (Sigma #M9309). For inoculation of HT-29 cells, cells were harvested, washed twice in PBS and resuspended at 5×10⁷ cells/mL in PBS. Cells were kept on ice until inoculation.

Animals were anaesthetized (isoflurane, 2-4% in ambient air supplemented with 100% O₂) prior to tumor inoculation. The tumor cells (100 μL suspension of either SK-RC-52 cells (2×10⁶ cells/animal) or HT-29 cells (5×10⁶ cells/animal)) were subcutaneously inoculated in the right flank using a 1-mL syringe equipped with a 27G needle. Tumor growth and animal weight were measured twice per week. Tumor size was measured by caliper and the volume was estimated using the following formula: 0.52×(length×width²). When tumor sizes reached 150-250 mm³, animals were randomized into groups (n=3) of similar average tumor volume and body weight.

Animals were injected intravenously with In-111-labeled compounds (single bolus, 22.3-31.2 MBq, ~1 nmol ligand, injection volume: 100 μL) in the lateral tail vein using a 29G syringe. Two additional groups of SK-RC-52 tumor-implanted mice were pretreated with intravenous injection of 100 μL 4% gelofusine, immediately before injection of [¹¹¹In]In-DPI-4452 and [¹¹¹In]In-DPI-4501, respectively.

Whole body SPECT/CT scanning (nanoScan SPECT/CT, Mediso) was performed under anesthesia (isoflurane, 2-4% in ambient air supplemented with 100% 02) at 1 h, 4 h, 24 h and 48 h after injection of [¹¹¹In]In-DPI-4452 or [¹¹¹In]In-DPI-4501 for the SK-RC-52 tumor experiments; in the experiment with the HT-29 tumor, the timepoints for SPECT-CT scan were: 1 h, 4 h, 24 h and 48 h after injection of [¹¹¹In]In-DPI-4452 or [¹¹¹In]In-DPI-4501. CT was performed with helical scan, 300 ms exposure, reconstruction resolution of 250 μm. SPECT was performed with multi-pinhole scan and a 30 s frame time. For the quantification of the radioactivity uptake, regions of interests (ROIs) were drawn over the tumor, kidney, liver, and heart (estimate of blood) identified on the images. Uptake was expressed as percent of injected dose per gram tissue (% ID/g).

### Results

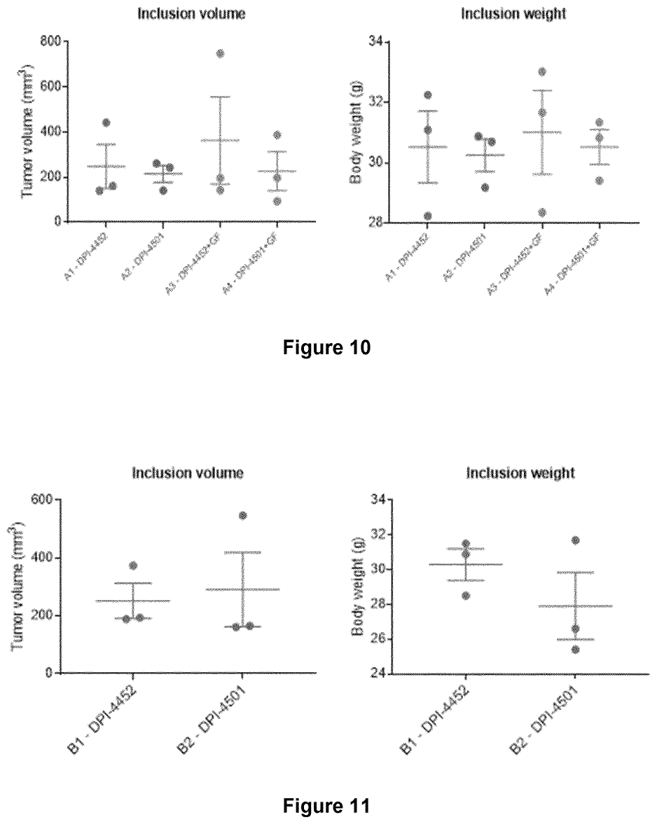

Animals with SK-RC-52 tumor (Groups A1-A4) were randomized in four groups with three animals per group at the day of dosing. No significant difference in tumor volume (p=0.80, one-way ANOVA) or animal body weight (P=0.96, one-way ANOVA) was observed between the groups. Tumor volume and body weight at randomization is presented in FIG. 10.

Animals with HT-29 (group B1-B2) were randomized in two groups with three animals per group at day−1 (the day before dosing). No significant difference in tumor volume (p=0.80, unpaired t-test) or animal body weight (P=0.32, unpaired t-test) was observed between the groups. Tumor volume and body weight at randomization is presented in FIG. 11.

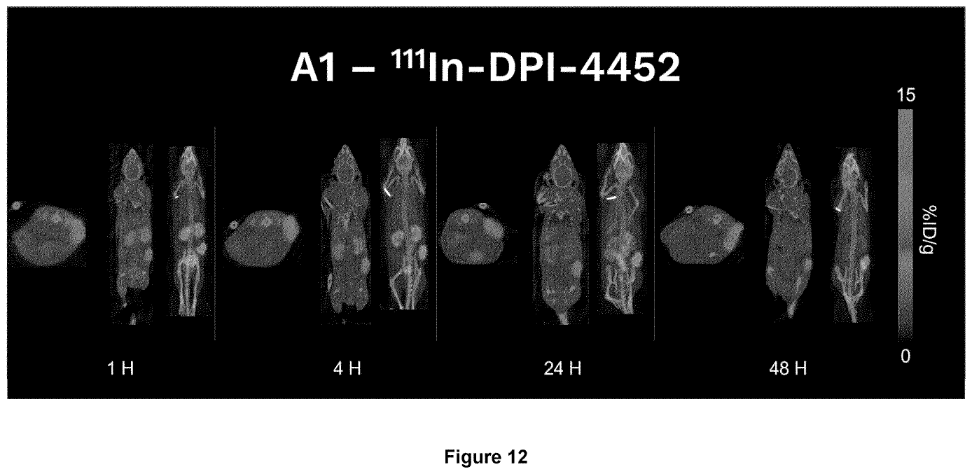

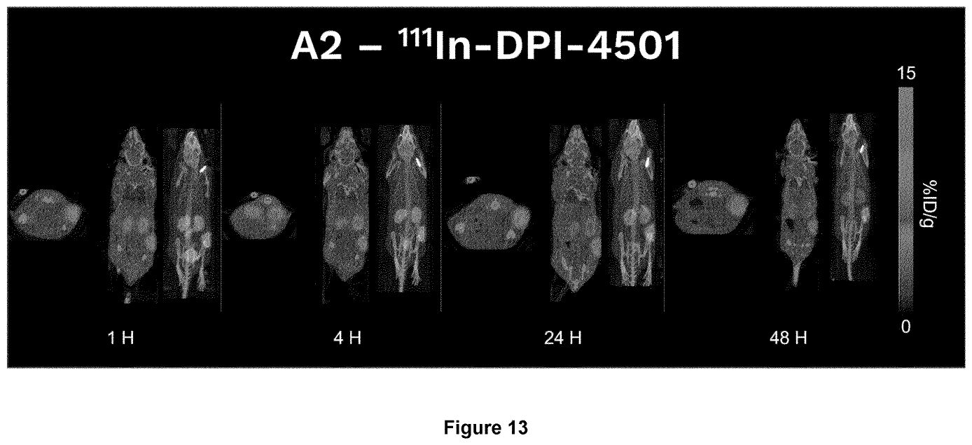

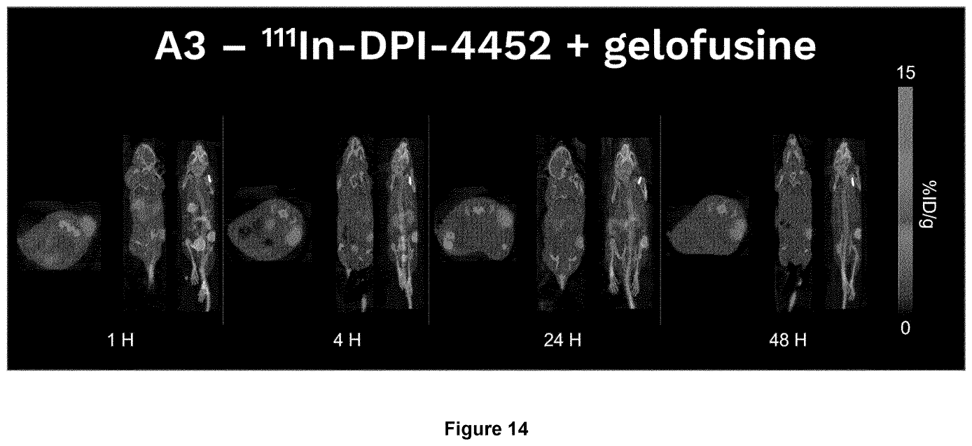

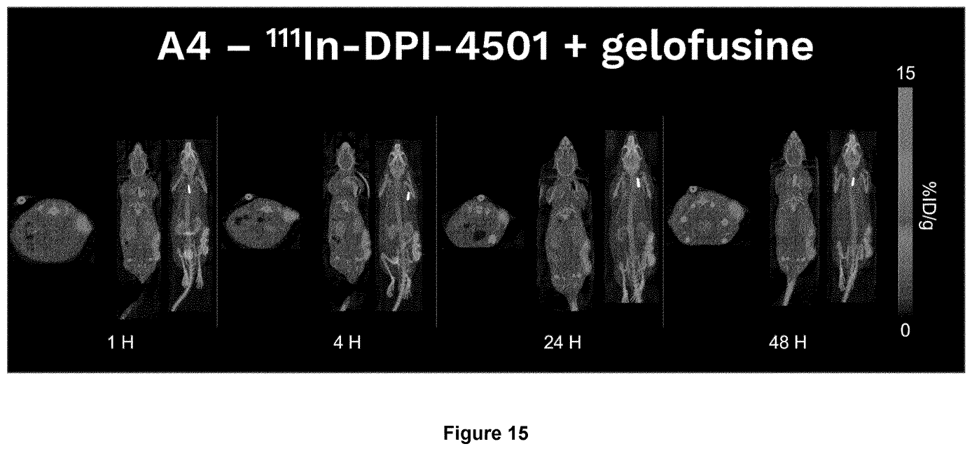

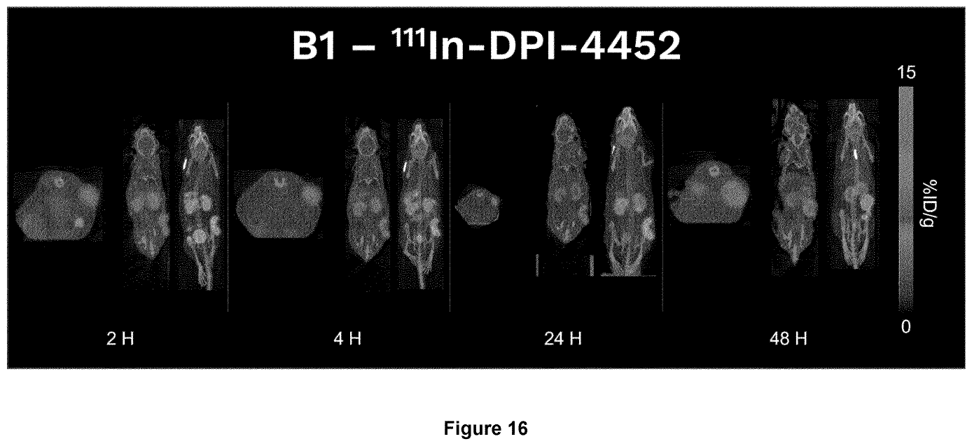

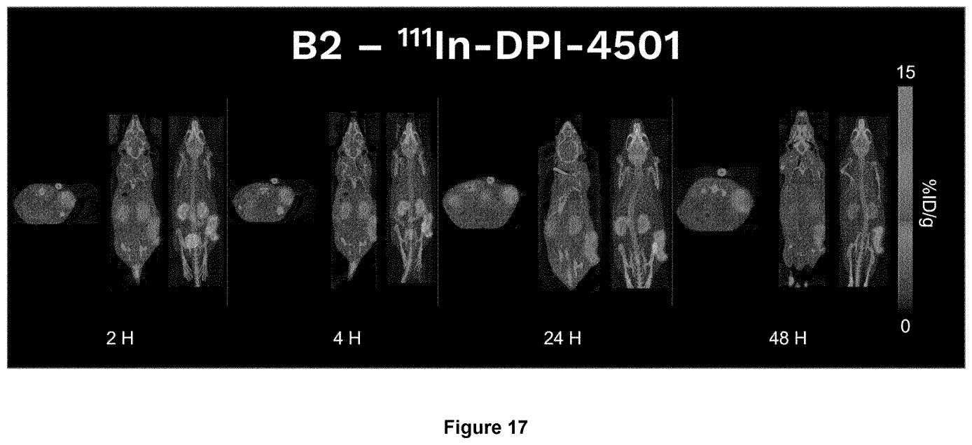

Both compounds were labeled with an In-111 incorporation of 90% on both radio-HPLC and radio-TLC for all labeling preparations. The radiochemical purity (RCP) was ≥95% on radio-HPLC and 100% on radio-TLC. After dosing of all the animals, RCP was found to be 95% for [¹¹¹In]In-DPI-4452 and 90% for [¹¹¹In]In-DPI-4501. SPECT/CT scans were collected at the above-mentioned time points. Representative axial and coronal images as well as maximum intensity projection (MIP) images of one mouse from each group are shown in FIG. 12-FIG. 17.

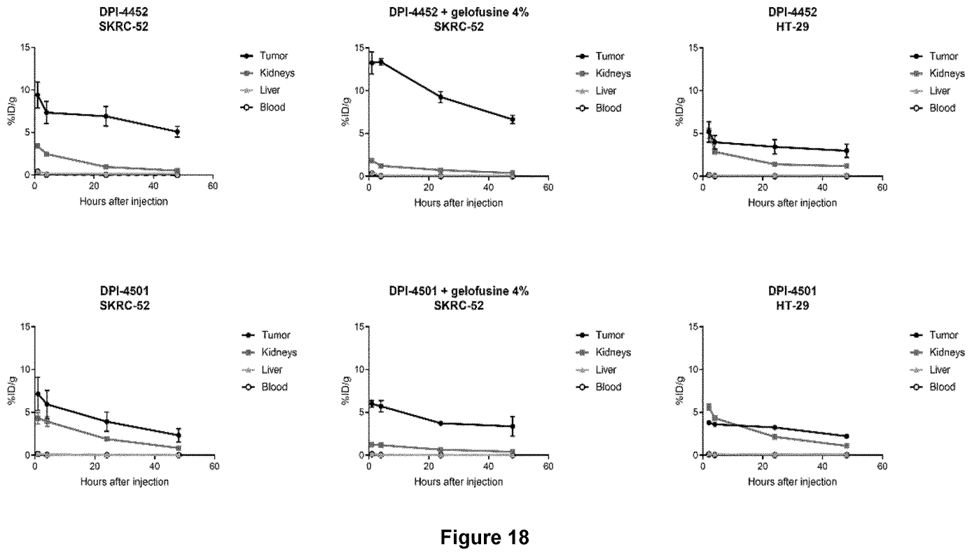

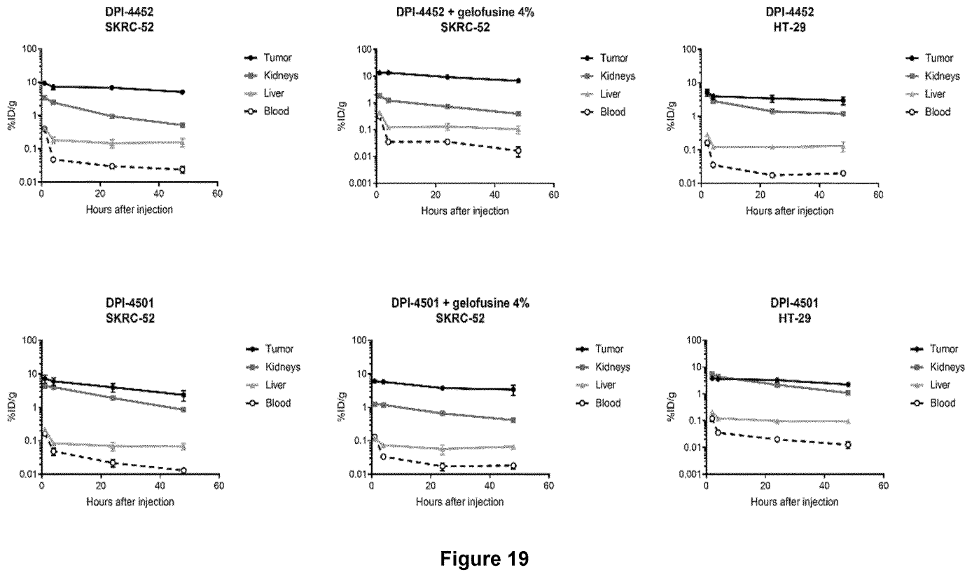

Distribution in tumor, kidneys, liver and blood measured as % ID/g are presented in FIGS. 18 and 19 on a linear and logarithmic scale, respectively. Uptake time profiles of [¹¹¹In]In-DPI-4452 and [¹¹¹In]In-DPI-5401 in SK-RC-52 and HT-29 tumors are presented in Table 6.

TABLE 6
Uptake time profiles of In-111-labeled DPI-4452 and DPI-4501 in SK-RC-52 and
HT-29 tumors. Uptake in SK-RC-52 tumor was compared with injection of 4% gelofusine
immediately before injection of compounds. N = 3/group, Mean ± SEM.

| | SK-RC-52 | | | | | |
|---|---|---|---|---|---|---|
| | | A3 | | A4 | HT-29 | |
| | A1 | DPI-4452 + | A2 | DPI-4501 + | B1 | B2 |
| | DPI-4452 | gelofusine | DPI-4501 | gelofusine | DPI-4452 | DPI-4501 |
| Peak Tumor Uptake | 9.42 ± 1.53 | 13.36 ± 0.39 | 7.17 ± 1.92 | 6.01 ± 0.39 | 5.21 ± 1.22 | 3.81 ± 0.26 |
| (% ID/g) | | | | | | |
| % remaining tumor | 73.3 ± 0.8 | 69.6 ± 6.6 | 54.3 ± 1.6 | 62.5 ± 1.9 | 65.8 ± 3.8 | 80 ± 4.4 |
| uptake at 24 h p.i. | | | | | | |
| Tumor/kidney uptake | 7.3 ± 1.3 | 12.7 ± 1.1 | 2.0 ± 0.1 | 5.8 ± 0.3 | 2.6 ± 0.8 | 1.5 ± 0.1 |
| ratio at 24 h p.i | | | | | | |
| Tumor/Liver uptake | 43.4 ± 16.2 | 111.2 ± 9.3 | 70.2 ± 17.0 | 81.4 ± 14.0 | 32.2 ± 6.1 | 29.8 ± 2.5 |
| ratio at 4 h p.i | | | | | | |
| Tumor/Blood uptake | 153.3 ± 7.5 | 428.3 ± 115.6 | 127.9 ± 20.3 | 178.6 ± 39.3 | 111.8 ± 18.3 | 104.5 ± 10.2 |
| ratio at 4 h p.i | | | | | | |
| Peak Kidney uptake | 3.43 ± 0.24 | 1.84 ± 0.14 | 4.33 ± 1.04 | 1.26 ± 0.12 | 5.37 ± 1.45 | 5.61 ± 0.57 |
| (% ID/g) | | | | | | |
| Kidney uptake at | 0.95 ± 0.05 | 0.73 ± 0.2 | 1.9 ± 0.4 | 0.65 ± 0.05 | 1.41 ± 0.11 | 2.16 ± 0.36 |
| 24 h p.i. (% ID/g) | | | | | | |
| Liver uptake at | 0.18 ± 0.04 | 0.12 ± 0.01 | 0.08 ± 0.01 | 0.07 ± 0.01 | 0.12 ± 0.005 | 0.12 ± 0.01 |
| 4 h p.i. (% ID/g) | | | | | | |
| Blood uptake at | 0.05 ± 0.01 | 0.03 ± 0.01 | 0.05 ± 0.01 | 0.03 ± 0.003 | 0.04 ± 0.002 | 0.04 ± 0.005 |
| 4 h p.i. (% ID/g) | | | | | | |
| Time to peak uptake | 1 | 4 | 1 | 1 | 2 | 2 |
| in tumor (h) | | | | | | |

For the SK-RC-52 tumor model, peak tumor uptake was observed at 1 hour post injection for [¹¹¹In]In-DPI-4452 (9.42±1.53% ID/g), [¹¹¹In]In-DPI-4501 (7.17±1.92% ID/g) and [¹¹¹In]In-DPI-4501+gelofusine (6.01±0.39% ID/g). For [¹¹¹In]In-DPI-4452+gelofusine peak tumor uptake was observed at 4 hours post injection (13.36±0.39% ID/g) (Table 6). Peak tumor uptake was higher for [¹¹¹In]In-DPI-4452 (9.42±1.53% ID/g) compared to [¹¹¹In]In-DPI-4501 (7.17±1.92% ID/g); however, the difference was not statistically significant (unpaired t-test, p=0.41). For the HT-29 tumor model, peak uptake was observed at 2 hours post injection for both [¹¹¹In]In-DPI-4452 and [¹¹¹In]In-DPI-4501. The peak tumor uptake in HT-29 was higher for [¹¹¹In]In-DPI-4452 (5.21±1.22% ID/g) than [¹¹¹In]In-DPI-4501 (3.81±0.26% ID/g); however, the difference was not statistically significant (unpaired t-test, p=0.33).

Injection of gelofusine immediately before injection of compounds in the SK-RC-52 tumor model resulted in an increased peak tumor uptake for [¹¹¹In]In-DPI-4452 (13.36±0.39% ID/g with gelofusine compared to 9.42±1.53% ID/g without gelofusine, p=0.07). Injection of gelofusine immediately before injection of [¹¹¹In]In-DPI-4501 had no significant impact on the peak tumor uptake (6.01±0.39% ID/g with gelofusine compared with 7.17±1.92% ID/g without gelofusine, p=0.59).

For both [¹¹¹In]In-DPI-4452 and [¹¹¹In]In-DPI-4501, injection of gelofusine immediately before injection of compounds significantly reduced peak kidney uptake (at 1-2 hours post injection) ([¹¹¹In]In-DPI-4452: 3.43±0.24% ID/g without gelofusine versus 1.84±0.14% ID/g with gelofusine, p=0.004; [¹¹¹In]In-DPI-4501: 4.33±1.04% ID/g without gelofusine versus 1.26±0.12% ID/g with gelofusine, p=0.04) and kidney uptake at 24 hours post injection ([¹¹¹In]In-DPI-4452: 0.95±0.05% ID/g without gelofusine versus 0.73±0.02% ID/g with gelofusine, p=0.01; [¹¹¹In]In-DPI-4501: 1.90±0.40% ID/g without gelofusine versus 0.65±0.05% ID/g with gelofusine, p=0.04).

Injection of 4% gelofusine immediately before injection of compounds resulted in an increase in tumor/kidney uptake ratio at 24 hours post injection, tumor/liver uptake ratio at 4 hours post-injection and tumor/blood uptake ratios at 4 hours post-injection for both DPI-4452 and DPI-4501 (Table 6).

### Conclusion

For both [¹¹¹In]In-DPI-4452 and [¹¹¹In]In-DPI-4501, tumor uptake was higher than kidney uptake; uptake in blood and liver had decreased to background level by 4 h p.i.; peak tumor uptake was typically 7-9% ID/g tissue, yet consistently slightly higher after injection of [¹¹¹In]In-DPI-4452 than [¹¹¹In]In-DPI-4501. The two tumor models SK-RC-52 and HT-29 gave similar results, yet uptake tended to be higher in SK-RC-52 tumor.

Injection of gelofusine immediately before injection of labeled compound in the SK-RC-52 tumor model resulted in: a significant decrease in kidney uptake for both [¹¹¹In]In-DPI-4452 and [¹¹¹In]In-DPI-4501; similar ([¹¹¹In]In-DPI-4501) or increased ([¹¹¹In]In-DPI-4452) tumor uptake; significant increase in tumor/kidney uptake ratio for both compounds, despite preferential uptake in tumor already without gelofusine.

### Example 5: Biodistribution Study in Healthy Dog

The biodistribution of [¹¹¹In]In-DPI-4452 and [¹¹¹In]In-DPI-4501 in male (n=2) and female (n=2) beagle dogs after i.v. (intravenous) administration was evaluated using in vivo SPECT-CT imaging at 1, 4 and 48 hours post injection. Blood pharmacokinetics and urine excretion data were determined from radioactivity concentration data determined by gamma-counting of respective samples.

The test compound mass dose level allometrically corresponded to a human dose of around 250 μg. The radioactivity dose was selected based on experience of the scanner sensitivity for In-111.

Before dosing, the dogs were fasted for a minimum of 6 hours, and a maximum of 24 hours before dosing due to sedation/anesthesia for SPECT/CT scanning and urine sampling at 1 and 4 hours after dosing. Furthermore, the animals were fasted before imaging and urine sampling at the 48-hour timepoint. The animals had ad libitum access to domestic quality drinking water.

Indium In-111 chloride (370 MBq/mL at activity reference time) was obtained from Curium and stored at room temperature before use. DPI-4452 and DPI-4501 were stored at −20° C. before use.

DPI-4452 (0.42 mg/mL (199 nmol/mL) solution in 0.1 M HEPES, pH 7) and DPI-4501 (0.40 mg/mL (203 nmol/mL) solution in 0.1 M HEPES, pH 7) were labeled at a molar activity of 15 MBq/nmol ligand (i.e. 115:1 stochiometric ratio of ligand:In-111) according to the following procedure:
In-111 (in 0.02 M HCl) and ligand (i.e., either DPI-4452 or DPI-4501) were mixed, and 1 M sodium acetate, pH 5.5 buffer was added (10% of the final volume).
The reaction mixture was stirred for 25 min at 80° C. at 600 rpm, and then quenched with 5 μL of 50 μM DTPA and cooled to room temperature.
The mixture was then diluted with water to a final volume of 10 mL and transferred onto the purification column (previously conditioned with 5 mL 99.9% EtOH followed by 5 mL water). The column was washed with 2 mL water, and then the final product was eluted from the column in EtOH solution (50%)
The sample was formulated in dPBS to the final concentration of 125 MBq/mL.

The formulations were kept at room temperature from labeling until dosing.

Quality control was conducted using one HPLC method and one TLC method. The HPLC QC method employed a Thermo Scientific Vanquish HPLC system including a UV detector set at 220 nm, a GABI Nova radiodetector, and an XBridge C18 3.5 μm 4.6×50 mm column. Chromatography was conducted at room temperature at a flow rate of 1.5 mL/min using a mobile phase consisting of A: 0.1% trifluoroacetic acid in water; and B: 0.1% trifluoroacetic acid in acetonitrile, according to the following linear gradient: 0-7 min from 5% B to 95% B; 7-8.5 min from 95% B to 5% B, 8.5-11 min: 5% B. The TLC QC method employed 11-cm long plates with an iTLC-SG stationary phase; the mobile phase was 0.1M citric acid, pH 5.4; the sample volume was 2 μL; the detector was a miniGita OFA Probe. The release criteria for the labeled compounds at EOS (end-of-synthesis) were 90% radiochemical purity from both the HPLC and the TLC methods.

For blood sampling, the dogs had a venflon inserted (BD 22G) in v. cephalica (front leg) or in v. saphena (hind leg). For dosing, the dogs had a venflon inserted on an opposite leg of the blood sampling, which was removed after dosing. The dogs received a single intravenous dose of 250 MBq In-111-labeled compound (36 and 38 nmol ligand of DPI-4452 and DPI-4501, respectively); the dose volume was 2 mL. The activity in the syringe was measured and the residual activity in the syringe and venflon was measured after dosing in a dose calibrator. The procedure for taking a sample of urine from live animals was cystocentesis with a 21 G cannula and a 5 mL syringe for females, whereas urine sample from males was taken through a urine catheter (placed 10-15 minutes prior to scheduled urine sampling timepoint). Collected urine was mixed to homogenize concentration.

Whole body SPECT/CT scanning (Clinical D670 SPECT/CT, GE) was performed under anesthesia 1, 4 and 48 hours after injection of the In-111-labeled compounds. The acquisition time enabled a minimum number of counts for good image resolution and quality while not exceeding the maximum time allowed for the animal to be in anesthesia for the 3 field of views needed to include the entire animal. During the CT part of the SPECT/CT scanning an intravenous infusion of an iodine-containing contrast medium (Iohexol, 300 mg/mL) was administrated as a 1 mL/kg volume and at a flow rate of 1 mL/sec to improve the delineation of the organs during image analysis.

The animals were transported in a sedated state to the scanner room on site. Sedation was achieved with 0.1-0.3 mg/kg i.m. (intramuscular)/i.v. (intravenous) Comfortan (methadone 10 mg/mL) and 0.002-0.01 mg/kg i.m./i.v. Dexdomitor® (dexmedetomidine 0.5 mg/mL). Then, anesthesia was induced by 3-6 mg/kg i.v. propofol (10 mg/mL). The dogs were intubated and connected to an anesthetic vaporizer and assigned 100% medicinal oxygen mixed with isoflurane (approx. 1.5-3%). The animals were sedated and anesthetized for 60-120 minutes.

For the quantification of the radioactivity uptake, a region of interest was drawn over eight (8) organs identified in the image data. The organs of interest were kidney, liver incl. gallbladder, gonads, bone marrow, lung with pleura, stomach, small intestine, and colon. Uptake was expressed as % ID/g (percent of injected dose per gram tissue) and SUV (standardized uptake value). Standardized uptake value (SUV) is widely used in clinical practice, calculated as the ratio of tissue radioactivity concentration (e.g. in kBq/ml) at a given time, and the administered dose per body weight (e.g. in MBq/kg).

Blood sampling was done at the following timepoints after dosing: 5 min, 10 min, 20 min, 30 min, 45 min, 1 h, 2 h, 4 h, 8 h, 24 h, 48 h and 72 h. Blood sampling was performed through the implanted venflon up to 4 h post injection, and then through v. jugularis with a cannula (BD 21G) and syringe (2 mL), or through v. cephalica with a cannula (21 G) if necessary due to temperament of dog, or anatomical reasons. Activity in blood samples was measured in a calibrated gamma counter (Hidex Automatics Gamma Counter) for 60 seconds using an energy window of 15-2047 keV.

Urine sampling was performed at 1, 4 and 48 hours post injection. The urine sampling in female dogs was performed using cystocenteses with a cannula (21 G) and syringe while the dog was under sedation or anesthesia. In male dogs, urine was sampled through a urine catheter which was placed during sedation. From each urine sample approx. 50 μL-0.5 mL of urine was transferred to 5 mL scintillation vials for activity measurement in the gamma counter for 60 seconds using an energy window of 15-2047 keV.

Blood half-life values were calculated by fitting bi-exponential equation to the measured blood activity concentrations.

### Results

DPI-4452 was labeled with an incorporation of ≥91% estimated from both radio-HPLC and radio-TLC. The radiochemical purity (RCP) at the end of synthesis (EOS) was ≥97% from radio-HPLC and 100% from radio-TLC. DPI-4501 was labeled with an incorporation of ≥92% estimated from both radio-HPLC and radio-TLC for the dosing of both female and male dogs, while RCP at EOS was ≥95% from radio-HPLC and 100% from radio-TLC. After dosing of all the animals, RCP was found to be ≥93% for [¹¹¹In]In-DPI-4452 and ≥91% for [¹¹¹In]In-DPI-4501.

The actual injected dose was 250 MBq (230 MBq in females dogs dosed with [¹¹¹In]In-DPI-4452) with 36 nmol total ligand. In the injected ¹¹¹In-labelled compound formulation, the ratio of In-complex to total ligand was 0.38% (0.41% in females dosed with [¹¹¹In]In-DPI-4452).

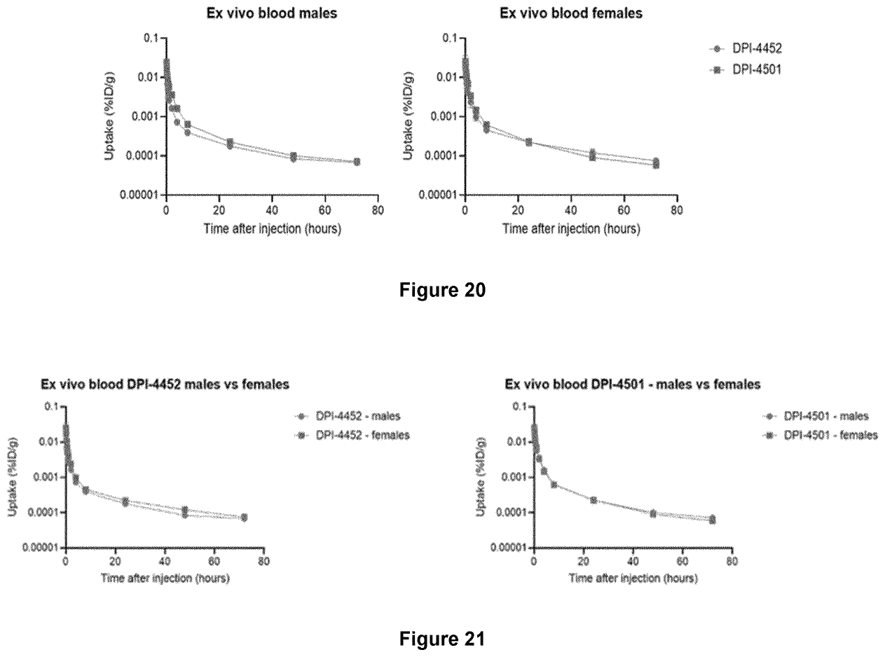

The time course of total radioactivity concentration in dog blood (expressed as % ID/g (% injected dose/g blood), corrected for radioactive decay) after a single IV dose of [¹¹¹In]In-DPI-4452 or [¹¹¹In]In-DPI-4501 is presented in FIG. 20 and FIG. 21, respectively.

There was no difference in blood pharmacokinetic profiles of males and females, after injection of either [¹¹¹In]In-DPI-4452 or [¹¹¹In]In-DPI-4501. The average blood kinetic profile of radioactivity after injection of [¹¹¹In]In-DPI-4452 followed an apparent biphasic course with one early distribution phase (timepoints from 5 minutes to 1 hour) characterized by a half-life (T½) of 5.43 min and one late elimination phase (timepoints from 24 hours to 72 hours) with T½=55.6 min. Similarly, the kinetic profile of radioactivity after injection of [¹¹¹In]In-DPI-4501 followed an apparent biphasic course with one early distribution phase (timepoints from 5 minutes to 1 hour) with T½=5.88 min and one late elimination phase (timepoints from 24 hours to 72 hours) with T½=68.9 min.

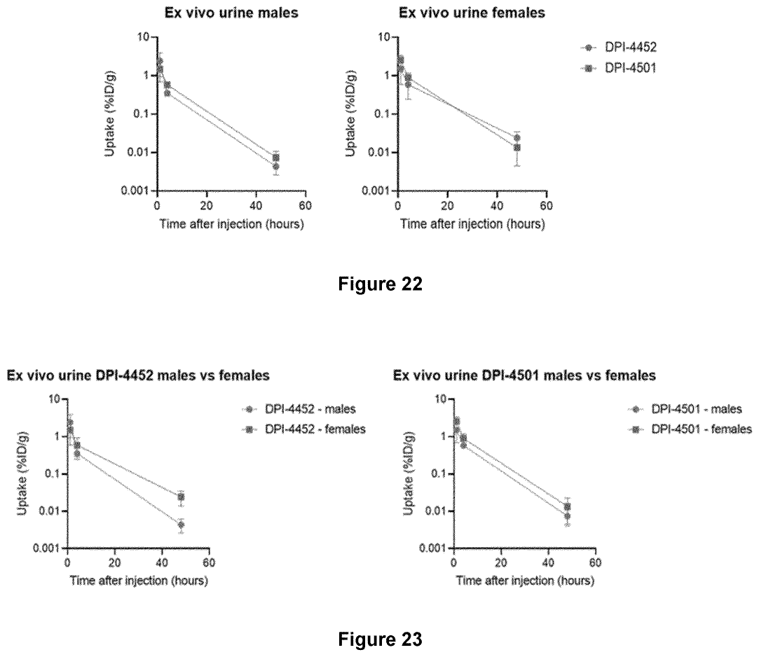

Radioactivity concentration in urine was determined at 1 h, 4 h and 4 8 h post injection (FIG. 22 and FIG. 23). Similar kinetic profiles were obtained for the two test compounds, i.e., concentration decreasing with time from about 2% ID/g down to low values of about 0.012% ID/g. No conclusion can be drawn from the apparent gender-related difference observed after injection of [¹¹¹In]In-DPI-4452 due to inter-individual differences in bladder emptying which could not be controlled in this experimental design.

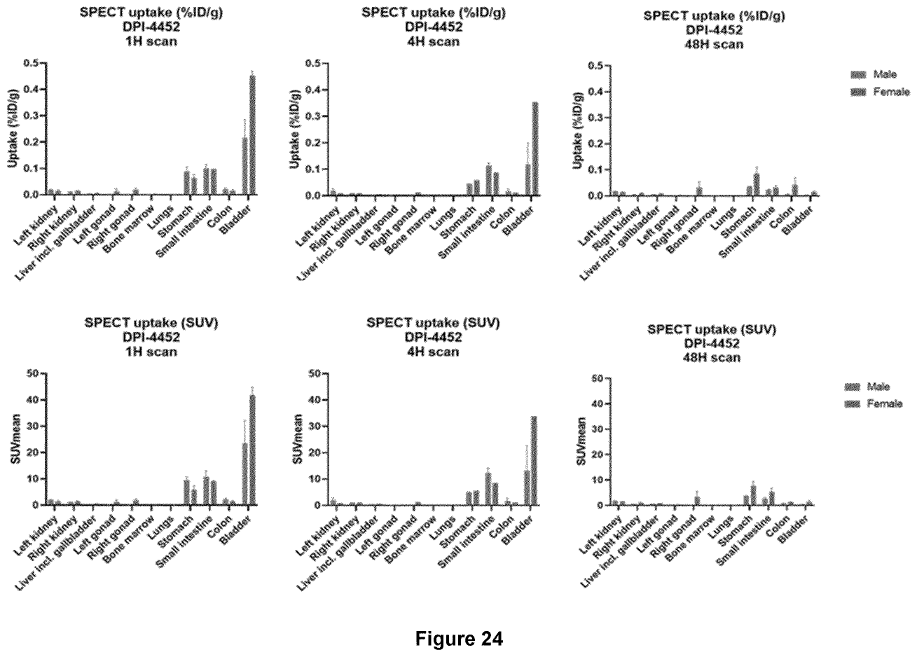

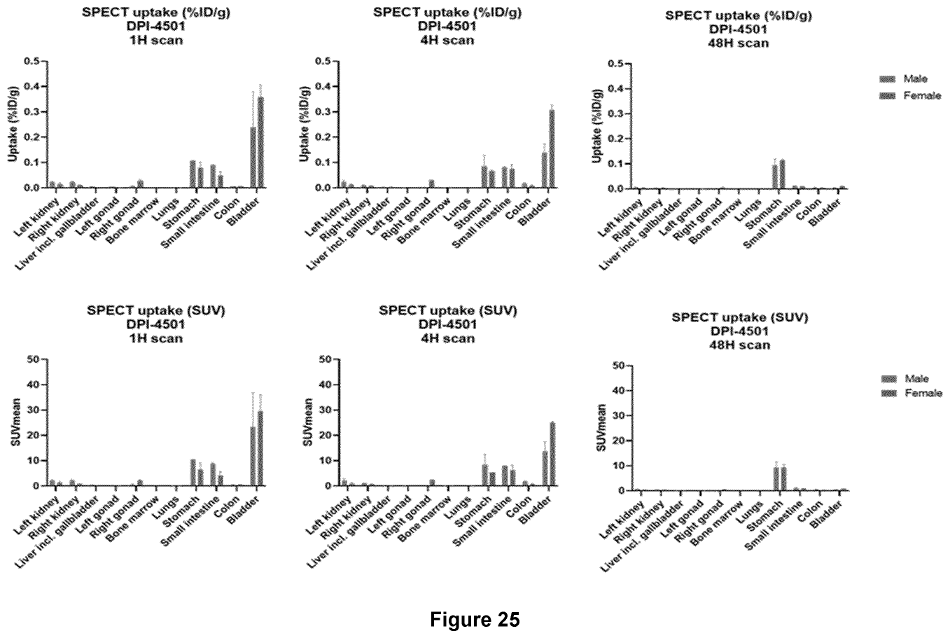

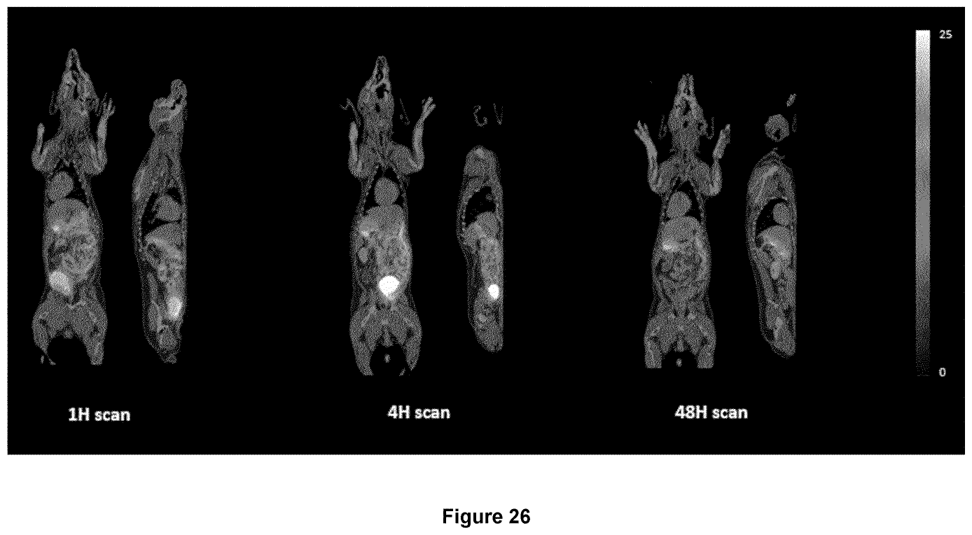

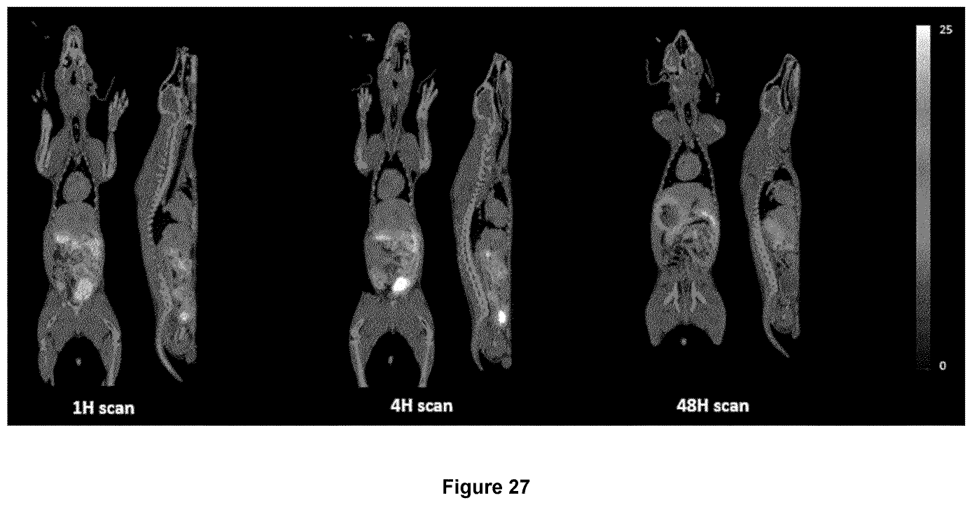

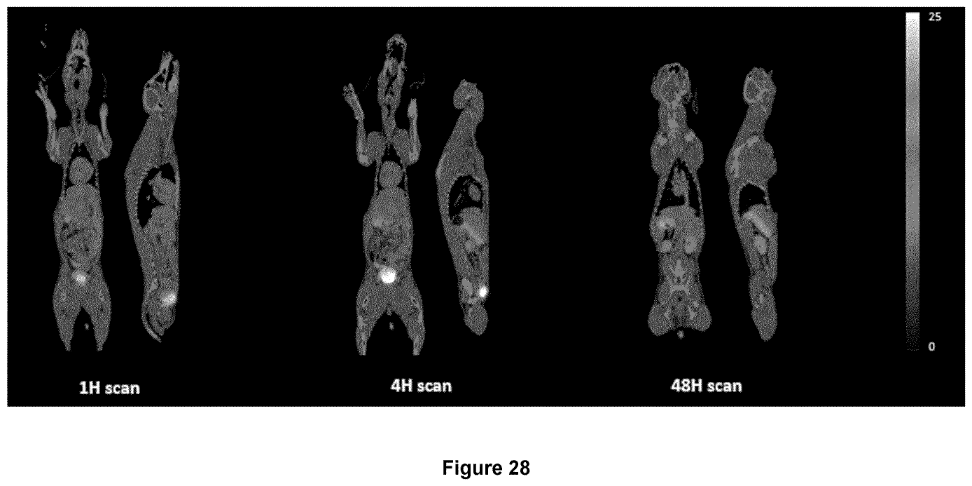

SPECT/CT scans were obtained at 1 h, 4 h and 48 h post-injection. Representative whole-body images are shown in FIG. 26 ([¹¹¹In]In-DPI-4452 in female dogs), FIG. 27 ([¹¹¹In]In-DPI-4452 in male dogs), FIG. 28 ([¹¹¹In]In-DPI-4501 in female dogs) and FIG. 29 ([¹¹¹In]In-DPI-4501 in male dogs). Organ uptake values are shown in FIG. 24 ([¹¹¹In]In-DPI-4452) and FIG. 25 ([¹¹¹In]In-DPI-4501).

Among the selected regions of interest, significant radioactivity uptake was observed in the bladder (0.2-0.5% ID/g or SUV 25-45 at 1 h p.i.), small intestine (ca. 0.1% ID/g or SUV ca. 10 at 1 h p.i.) and the stomach (ca. 0.1% ID/g or SUV ca. 10 at 1 h p.i.), whereas uptake in the other organs was very low, typically below 0.2% ID/g or SUV below 2, which may be considered as background level (gonads, kidneys, liver with gallbladder, colon, bone marrow, lungs).

Radioactivity uptake in the organs tended to decrease with time, from 1 h to 4 h and to 48 h post-injection in the bladder and in the small intestine, whereas no trend was seen in the organs with background (very low) uptake levels, and sustained uptake was observed in the stomach.

The high level of radioactivity in the bladder is likely resulting from extensive excretion of radioactivity in urine. In females, average bladder uptake tended to be approximatively twice as high as in males at 1-4 h p.i., yet variability was high, in line with inter-individual variability in the presence of not-yet excreted urine.

The high levels of radioactivity in the small intestine and stomach likely result from the presence of naturally expressed target CAIX in those organs in the dog, since both test compounds have been demonstrated to bind dog CAIX with similar potency to human CAIX (see Example 7), and expression of CAIX in those organs in the dog has been documented [The Human Protein Atlas, https://www.proteinatlas.org/ENSG00000107159-CA9/tissue]. Levels observed in the colon were low and not significantly different from background.

As apparent in FIG. 24 and FIG. 25, the right gonad of the females tended to have markedly higher uptake than the left gonad, which has been interpreted as being due to spillover from the GI tract and stomach rather than specific uptake in the gonads. The differences here should therefore be anticipated to have origin in the placement of the right gonad rather than specific target binding.

### Example 6: Dosimetry Study Based on Dog Biodistribution Data

Dosimetry of ¹¹¹In radiation based on organ uptake data from the above dog biodistribution study was conducted as follows. The area under the time activity curves was calculated using linear interpolation between datapoints, assuming residual activity at the last timepoint to decay fully in the tissue. The number of disintegrations per gram tissue per administered MBq was calculated and extrapolated to human using the % kg/g method (Kirschner et al., J. Nucl. Med. 1975, 16(3), 248-249) using individual animal body weights, ICRP89 human phantom body weights and organ masses to calculate the number of disintegrations per human organ (ICRP 89, 2002, Basic Anatomical and Physiological Data for Use in Radiological Protection Reference Values. ICRP Publication 89. Ann. ICRP 32 (3-4)), and according to the following formula:

$$[ {( \frac{\%}{{g}_{organ}} )}_{animal} \times {( {kg}_{TBweight} )}_{animal} ] \times {( \frac{{g}_{organ}}{{kg}_{TBweight}} )}_{human} = {( \frac{\%}{organ} )}_{human}$$

This was converted to residence time by division by 3.6E9 dis/MBq/h (number of decays from a constant activity of 1 MBq during a period of 1 h) and entered into OLINDA/EXM 2.0 to calculate human absorbed doses. Output ICRP 103 ED is the effective dose contribution from the individual organs/compartments. Finally, the effective dose is calculated as the average dose to males and females (MIRD Pamphlet 21; Bolch et al. J. Nucl. Med. 2009, 50(3), 477-484).

Estimation of the dosimetry of the ¹⁷⁷Lu-labeled analogue was performed by re-calculation of the individual data points, based on the difference in half-life between In-111 and Lu-177. Then, the subsequent steps were identical to ¹¹¹In dosimetry. Organ-related maximum tolerated dose values established for external beam radiotherapy (From Table 3 in Tolerance of Normal Tissue to Therapeutic Radiation, Dr. Emami B, 2013) were used to calculate the maximum allowed injected radioactivity dose (a conservative approach).

Finally, at the projected maximum allowed ¹⁷⁷Lu injected radioactivity dose, the range of possible radiation doses delivered to a human tumor was estimated based on tumor uptake derived from a xenografted mouse study (described above in this document). Since the relative sizes of the xenograft tumor and the whole mouse body are not matching the situation of a human patient with tumor, two different approaches of extrapolation of xenograft tumor dosimetry were used and yielded a range of limit values. One approach consisted in keeping constant % ID (a typically reported method, see Biodistribution and radiation dosimetry of radio-iodinated hypericin as a cancer therapeutic, Cona et al. 2013, and Enhancing Treatment Efficacy of ¹⁷⁷Lu-PSMA-617 with the Conjugation of an Albumin-Binding Motif: Preclinical Dosimetry and Endoradiotherapy Studies, Kuo et al., Molecular Pharmaceutics 2018, 15(11), 5183-5191), however leading to overestimation of absorbed dose in small tumors. The other approach consisted in assuming a constant activity concentration (% ID/g) in the tumor tissue, which leads to an underestimation of dose absorbed in small tumors. Thus, the real value of radiation dose absorbed in the tumor will most likely lie between these two different extreme estimates of tumor absorbed radiation dose. A tumor size of 11 g was considered, as the average tumor weight of 5 randomly chosen actual patients in the clinic from a personal discussion between the study director and a nuclear physicist. The purpose of this preclinical tumor dosimetry was to check whether a maximum allowed injected dose of [¹⁷⁷Lu]Lu-DPI-4452 or [¹⁷⁷Lu]Lu-DPI-4501 radioactivity enables to deliver a radiation dose high enough to cause significant damage to the tumor, i.e., at least 50 Gy.

### Results

The radiation residence time in the different organs was calculated for input into OLINDA (Table 7 and Table 8, for [¹¹¹In]In-DPI-4452; Table 9 and Table 10, for [¹¹¹In]In-DPI-4501). Radiation doses absorbed in human organs were extrapolated using OLINDA (Table 11 and Table 12, for [¹¹¹In]In-DPI-4452; Table 13 and Table 14, for [¹¹¹In]In-DPI-4501). The resulting effective doses were 1.03×10⁻¹ mSv/MBq and 8.44×10⁻² mSv/MBq, respectively (Table 15 and Table 16).

TABLE 7
Input parameters for the male dose
calculation for [¹¹¹In]In-DPI-4452.

| Organ | Mass(g) | Residence time (h) |
|---|---|---|
| Adrenals | 14 | 0.00E+00 |
| Brain | 1450 | 0.00E+00 |
| Esophagus | 40 | 0.00E+00 |
| Eyes | 15 | 0.00E+00 |
| Gallbladder Contents | 58 | 3.85E−02 |
| Left Colon | 75 | 9.73E−02 |
| Small Intestine | 350 | 2.30E+00 |
| Stomach Contents | 250 | 1.36E+00 |
| Right Colon | 150 | 1.95E−01 |
| Rectum | 75 | 9.73E−02 |
| Heart Contents | 510 | 1.70E−02 |
| Heart Wall | 330 | 0.00E+00 |
| Kidneys | 310 | 5.05E−01 |
| Liver | 1800 | 1.19E+00 |
| Lungs | 1200 | 2.55E−01 |
| Pancreas | 140 | 0.00E+00 |
| Prostate | 17 | 0.00E+00 |
| Salivary Glands | 85 | 0.00E+00 |
| Red Marrow | 1170 | 8.24E−02 |
| Cortical Bone | 4400 | 0.00E+00 |
| Trabecular Bone | 1100 | 0.00E+00 |
| Spleen | 150 | 0.00E+00 |
| Testes | 35 | 2.77E−03 |
| Thymus | 25 | 0.00E+00 |
| Thyroid | 20 | 0.00E+00 |
| Urinary Bladder Contents | 211 | 1.09E+00 |
| Total Body | 73000 | 4.09E+01 |

TABLE 8
Input parameters for the female dose calculation
for compound [¹¹¹In]In-DPI-4452.

| Organ | Mass(g) | Residence time (h) |
|---|---|---|
| Adrenals | 13 | 0.00E+00 |
| Brain | 1300 | 0.00E+00 |
| Breasts | 500 | 0.00E+00 |
| Esophagus | 35 | 0.00E+00 |
| Eyes | 15 | 0.00E+00 |
| Gallbladder Contents | 48 | 5.53E−02 |
| Left Colon | 80 | 1.40E−01 |
| Small Intestine | 280 | 2.65E+00 |
| Stomach Contents | 230 | 2.68E+00 |
| Right Colon | 160 | 2.80E−01 |
| Rectum | 80 | 1.40E−01 |
| Heart Contents | 370 | 1.79E−02 |
| Heart Wall | 250 | 0.00E+00 |
| Kidneys | 275.5 | 4.72E−01 |
| Liver | 1400 | 1.61E+00 |
| Lungs | 950 | 3.65E−01 |
| Ovaries | 11 | 3.26E−03 |
| Pancreas | 120 | 0.00E+00 |
| Salivary Glands | 70 | 0.00E+00 |
| Red Marrow | 900 | 1.07E−01 |
| Cortical Bone | 3200 | 0.00E+00 |
| Trabecular Bone | 800 | 0.00E+00 |
| Spleen | 130 | 0.00E+00 |
| Thymus | 20 | 0.00E+00 |
| Thyroid | 17 | 0.00E+00 |
| Urinary Bladder Contents | 160 | 1.50E+00 |
| Uterus | 80 | 0.00E+00 |
| Total Body | 60000 | 2.72E+01 |

TABLE 9
Input parameters for the male dose calculation
for compound [¹¹¹In]In-DPI-4501.

| | | Kinetics Value |
|---|---|---|
| Source Organ Name | Mass [g] | [MBq-h/MBq] |
| Adrenals | 14 | 0.00E+00 |
| Brain | 1450 | 0.00E+00 |
| Esophagus | 40 | 0.00E+00 |
| Eyes | 15 | 0.00E+00 |
| Gallbladder Contents | 58 | 1.51E−02 |
| Left Colon | 75 | 7.61E−02 |
| Small Intestine | 350 | 1.35E+00 |
| Stomach Contents | 250 | 3.02E+00 |
| Right Colon | 150 | 1.52E−01 |
| Rectum | 75 | 7.61E−02 |
| Heart Contents | 510 | 2.51E−02 |
| Heart Wall | 330 | 0.00E+00 |
| Kidneys | 310 | 3.29E−01 |
| Liver | 1800 | 4.68E−01 |
| Lungs | 1200 | 4.77E−02 |
| Pancreas | 140 | 0.00E+00 |
| Prostate | 17 | 0.00E+00 |
| Salivary Glands | 85 | 0.00E+00 |
| Red Marrow | 1170 | 2.88E−02 |
| Cortical Bone | 4400 | 0.00E+00 |
| Trabecular Bone | 1100 | 0.00E+00 |
| Spleen | 150 | 0.00E+00 |
| Testes | 35 | 1.45E−03 |
| Thymus | 25 | 0.00E+00 |
| Thyroid | 20 | 0.00E+00 |
| Urinary Bladder Contents | 211 | 1.62E+00 |
| Total Body | 73000 | 1.74E+01 |

TABLE 10
Input parameters for the female dose calculation
for compound [¹¹¹In]In-DPI-4501.

| | | Kinetics Value |
|---|---|---|
| Source Organ Name | Mass [g] | [MBq-h/MBq] |
| Adrenals | 13 | 0.00E+00 |
| Brain | 1300 | 0.00E+00 |
| Breasts | 500 | 0.00E+00 |
| Esophagus | 35 | 0.00E+00 |
| Eyes | 15 | 0.00E+00 |
| Gallbladder Contents | 48 | 9.73E−03 |
| Left Colon | 80 | 5.21E−02 |
| Small Intestine | 280 | 8.76E−01 |
| Stomach Contents | 230 | 2.85E+00 |
| Right Colon | 160 | 1.04E−01 |
| Rectum | 80 | 5.21E−02 |
| Heart Contents | 370 | 1.86E−02 |
| Heart Wall | 250 | 0.00E+00 |
| Kidneys | 275,5 | 1.98E−01 |
| Liver | 1400 | 2.84E−01 |
| Lungs | 950 | 4.22E−02 |
| Ovaries | 11 | 8.86E−04 |
| Pancreas | 120 | 0.00E+00 |
| Salivary Glands | 70 | 0.00E+00 |
| Red Marrow | 900 | 3.30E−02 |
| Cortical Bone | 3200 | 0.00E+00 |
| Trabecular Bone | 800 | 0.00E+00 |
| Spleen | 130 | 0.00E+00 |
| Thymus | 20 | 0.00E+00 |
| Thyroid | 17 | 0.00E+00 |
| Urinary Bladder Contents | 160 | 2.02E+00 |
| Uterus | 80 | 0.00E+00 |
| Total Body | 60000 | 1.28E+01 |

TABLE 11
Olinda output data for the human male ICRP-89 phantom per MBq administered
[¹¹¹In]In-DPI-4452. Values are given in mGy/MBq for Beta, Gamma
and Total, whereas ICRP-103 ED and Effective dose is given in mSv/MBq.

| Target Organ | Alpha | Beta | Gamma | Total | ICRP-103ED |
|---|---|---|---|---|---|
| Adrenals | 0.00E+00 | 1.15E−02 | 9.23E−02 | 1.04E−01 | 9.58E−04 |
| Brain | 0.00E+00 | 1.13E−02 | 4.68E−02 | 5.80E−02 | 5.80E−04 |
| Esophagus | 0.00E+00 | 1.13E−02 | 6.52E−02 | 7.65E−02 | 3.06E−03 |
| Eyes | 0.00E+00 | 1.13E−02 | 4.68E−02 | 5.80E−02 | 0.00E+00 |
| Gallbladder Wall | 0.00E+00 | 1.80E−02 | 8.64E−02 | 1.04E−01 | 9.64E−04 |
| Left colon | 0.00E+00 | 2.43E−02 | 1.07E−01 | 1.31E−01 | 6.37E−03 |
| Small Intestine | 0.00E+00 | 7.72E−02 | 1.42E−01 | 2.19E−01 | 2.02E−03 |
| Stomach Wall | 0.00E+00 | 6.60E−02 | 1.49E−01 | 2.15E−01 | 2.58E−02 |
| Right colon | 0.00E+00 | 2.43E−02 | 9.18E−02 | 1.16E−01 | 5.63E−03 |
| Rectum | 0.00E+00 | 2.43E−02 | 9.68E−02 | 1.21E−01 | 2.78E−03 |
| Heart Wall | 0.00E+00 | 1.16E−02 | 6.11E−02 | 7.27E−02 | 6.71E−04 |
| Kidneys | 0.00E+00 | 3.25E−02 | 9.61E−02 | 1.29E−01 | 1.19E−03 |
| Liver | 0.00E+00 | 1.33E−02 | 7.23E−02 | 8.56E−02 | 3.43E−03 |
| Lungs | 0.00E+00 | 4.23E−03 | 5.41E−02 | 5.83E−02 | 7.00E−03 |
| Pancreas | 0.00E+00 | 1.13E−02 | 1.24E−01 | 1.35E−01 | 1.25E−03 |
| Prostate | 0.00E+00 | 1.13E−02 | 8.99E−02 | 1.01E−01 | 4.67E−04 |
| Salivary Glands | 0.00E+00 | 1.13E−02 | 5.86E−02 | 6.99E−02 | 6.99E−04 |
| Red Marrow | 0.00E+00 | 1.00E−02 | 5.70E−02 | 6.70E−02 | 8.04E−03 |
| Osteogenic Cells | 0.00E+00 | 1.55E−02 | 9.55E−02 | 1.11E−01 | 1.11E−03 |
| Spleen | 0.00E+00 | 1.13E−02 | 8.12E−02 | 9.25E−02 | 8.54E−04 |
| Testes | 0.00E+00 | 1.57E−03 | 4.83E−02 | 4.98E−02 | 1.99E−03 |
| Thymus | 0.00E+00 | 1.13E−02 | 5.38E−02 | 6.51E−02 | 6.01E−04 |
| Thyroid | 0.00E+00 | 1.13E−02 | 5.70E−02 | 6.83E−02 | 2.73E−03 |
| Urinary Bladder Wall | 0.00E+00 | 6.32E−02 | 1.51E−01 | 2.14E−01 | 8.56E−03 |
| Total Body | 0.00E+00 | 1.13E−02 | 5.86E−02 | 7.18E−02 | 0.00E+00 |
| Effective Dose | 8.68E−02 | | | | |

TABLE 12
Olinda output data for the human female ICRP-89 phantom per MBq administered
[¹¹¹In]In-DPI-4452. Values are given in mGy/MBq for Beta, Gamma and
Total, whereas ICRP-103 ED and Effective dose is given in mSv/MBq.

| Target Organ | Alpha | Beta | Gamma | Total | ICRP-103ED |
|---|---|---|---|---|---|
| Adrenals | 0.00E+00 | 9.28E−03 | 1.14E−01 | 1.23E−01 | 1.14E−03 |
| Brain | 0.00E+00 | 9.09E−03 | 3.87E−02 | 4.78E−02 | 4.78E−04 |
| Breasts | 0.00E+00 | 9.09E−03 | 3.56E−02 | 4.47E−02 | 5.36E−03 |
| Esophagus | 0.00E+00 | 9.09E−03 | 5.55E−02 | 6.46E−02 | 2.59E−03 |
| Eyes | 0.00E+00 | 9.09E−03 | 3.88E−02 | 4.79E−02 | 0.00E+00 |
| Gallbladder Wall | 0.00E+00 | 2.07E−02 | 1.09E−01 | 1.30E−01 | 1.20E−03 |
| Left colon | 0.00E+00 | 2.67E−02 | 1.05E−01 | 1.32E−01 | 6.40E−03 |
| Small Intestine | 0.00E+00 | 1.04E−01 | 1.56E−01 | 2.60E−01 | 2.40E−03 |
| Stomach Wall | 0.00E+00 | 1.26E−01 | 2.62E−01 | 3.88E−01 | 4.66E−02 |
| Right colon | 0.00E+00 | 2.67E−02 | 9.74E−02 | 1.24E−01 | 6.02E−03 |
| Rectum | 0.00E+00 | 2.67E−02 | 1.13E−01 | 1.40E−01 | 3.22E−03 |
| Heart Wall | 0.00E+00 | 9.59E−03 | 6.13E−02 | 7.09E−02 | 6.54E−04 |
| Kidneys | 0.00E+00 | 3.41E−02 | 1.04E−01 | 1.38E−01 | 1.28E−03 |
| Liver | 0.00E+00 | 2.31E−02 | 9.60E−02 | 1.19E−01 | 4.76E−03 |
| Lungs | 0.00E+00 | 7.63E−03 | 5.94E−02 | 6.70E−02 | 8.05E−03 |
| Ovaries | 0.00E+00 | 5.85E−03 | 8.98E−02 | 9.56E−02 | 3.83E−03 |
| Pancreas | 0.00E+00 | 9.12E−03 | 1.36E−01 | 1.45E−01 | 1.34E−03 |
| Salivary Glands | 0.00E+00 | 9.09E−03 | 4.13E−02 | 5.04E−02 | 5.04E−04 |
| Red Marrow | 0.00E+00 | 8.74E−03 | 5.36E−02 | 6.23E−02 | 7.48E−03 |
| Osteogenic Cells | 0.00E+00 | 9.57E−03 | 8.35E−02 | 9.30E−02 | 9.30E−04 |
| Spleen | 0.00E+00 | 9.13E−03 | 1.13E−01 | 1.22E−01 | 1.13E−03 |
| Thymus | 0.00E+00 | 9.13E−03 | 5.01E−02 | 5.92E−02 | 5.47E−04 |
| Thyroid | 0.00E+00 | 9.09E−03 | 4.20E−02 | 5.11E−02 | 2.05E−03 |
| Urinary Bladder Wall | 0.00E+00 | 1.03E−01 | 1.55E−01 | 2.58E−01 | 1.05E−02 |
| Uterus | 0.00E+00 | 9.09E−03 | 1.20E−01 | 1.29E−01 | 5.94E−04 |
| Total Body | 0.00E+00 | 1.24E−02 | 5.66E−02 | 6.90E−02 | 0.00E+00 |
| Effective Dose | 1.19E−01 | | | | |

TABLE 13
Olinda output data for the human male ICRP-89 phantom per MBq administered
[¹¹¹In]In-DPI-4501. Values are given in mGy/MBq for Beta, Gamma
and Total, whereas ICRP-103 ED and Effective dose is given in mSv/MBq.

| Target Organ | Alpha | Beta | Gamma | Total | ICRP-103 ED |
|---|---|---|---|---|---|
| Adrenals | 0.00E+00 | 4.94E−03 | 5.94E−02 | 6.44E−02 | 5.94E−04 |
| Brain | 0.00E+00 | 4.80E−03 | 2.00E−02 | 2.48E−02 | 2.48E−04 |
| Esophagus | 0.00E+00 | 4.80E−03 | 4.90E−02 | 5.38E−02 | 2.15E−03 |
| Eyes | 0.00E+00 | 4.80E−03 | 2.00E−02 | 2.48E−02 | 0.00E+00 |
| Gallbladder Wall | 0.00E+00 | 7.43E−03 | 4.44E−02 | 5.18E−02 | 4.78E−04 |
| Left colon | 0.00E+00 | 1.50E−02 | 6.95E−02 | 8.45E−02 | 4.10E−03 |
| Small Intestine | 0.00E+00 | 4.34E−02 | 8.21E−02 | 1.26E−01 | 1.16E−03 |
| Stomach Wall | 0.00E+00 | 1.26E−01 | 2.32E−01 | 3.58E−01 | 4.30E−02 |
| Right colon | 0.00E+00 | 1.50E−02 | 4.90E−02 | 6.40E−02 | 3.10E−03 |
| Rectum | 0.00E+00 | 1.50E−02 | 5.90E−02 | 7.40E−02 | 1.70E−03 |
| Heart Wall | 0.00E+00 | 5.29E−03 | 4.61E−02 | 5.14E−02 | 4.74E−04 |
| Kidneys | 0.00E+00 | 2.12E−02 | 5.73E−02 | 7.85E−02 | 7.24E−04 |
| Liver | 0.00E+00 | 5.21E−03 | 3.92E−02 | 4.44E−02 | 1.78E−03 |
| Lungs | 0.00E+00 | 7.94E−04 | 3.36E−02 | 3.44E−02 | 4.13E−03 |
| Pancreas | 0.00E+00 | 4.80E−03 | 1.21E−01 | 1.25E−01 | 1.16E−03 |
| Prostate | 0.00E+00 | 4.80E−03 | 5.69E−02 | 6.17E−02 | 2.85E−04 |
| Salivary Glands | 0.00E+00 | 4.80E−03 | 2.53E−02 | 3.01E−02 | 3.01E−04 |
| Red Marrow | 0.00E+00 | 4.21E−03 | 3.00E−02 | 3.42E−02 | 4.10E−03 |
| Osteogenic Cells | 0.00E+00 | 6.55E−03 | 4.68E−02 | 5.33E−02 | 5.33E−04 |
| Spleen | 0.00E+00 | 4.81E−03 | 6.11E−02 | 6.59E−02 | 6.08E−04 |
| Testes | 0.00E+00 | 8.19E−04 | 2.44E−02 | 2.52E−02 | 1.01E−03 |
| Thymus | 0.00E+00 | 4.80E−03 | 2.71E−02 | 3.19E−02 | 2.94E−04 |
| Thyroid | 0.00E+00 | 4.80E−03 | 2.58E−02 | 3.06E−02 | 1.22E−03 |
| Urinary Bladder Wall | 0.00E+00 | 8.20E−02 | 1.58E−01 | 2.40E−01 | 9.59E−03 |
| Total Body | 0.00E+00 | 6.78E−03 | 2.86E−02 | 3.53E−02 | 0.00E+00 |
| Effective Dose | 8.27E−02 | | | | |

TABLE 14
Olinda output data for the human female ICRP-89 phantom per MBq administered
[¹¹¹In]In-DPI-4501. Values are given in mGy/MBq for Beta, Gamma and
Total, whereas ICRP-103 ED and Effective dose is given in mSv/MBq.

| Target Organ | Alpha | Beta | Gamma | Total | ICRP-103 ED |
|---|---|---|---|---|---|
| Adrenals | 0.00E+00 | 4.33E−03 | 6.62E−02 | 7.05E−02 | 6.15E−04 |
| Brain | 0.00E+00 | 4.27E−03 | 1.82E−02 | 2.25E−02 | 2.25E−04 |
| Breasts | 0.00E+00 | 4.27E−03 | 1.76E−02 | 2.19E−02 | 2.62E−03 |
| Esophagus | 0.00E+00 | 4.27E−03 | 2.92E−02 | 3.35E−02 | 1.34E−03 |
| Eyes | 0.00E+00 | 4.27E−03 | 1.82E−02 | 2.25E−02 | 0.00E+00 |
| Gallbladder Wall | 0.00E+00 | 6.31E−03 | 4.37E−02 | 5.00E−02 | 4.61E−04 |
| Left colon | 0.00E+00 | 1.08E−02 | 5.81E−02 | 6.89E−02 | 3.34E−03 |
| Small Intestine | 0.00E+00 | 3.57E−02 | 7.07E−02 | 1.06E−01 | 9.82E−04 |
| Stomach Wall | 0.00E+00 | 1.29E−01 | 2.40E−01 | 3.69E−01 | 4.43E−02 |
| Right colon | 0.00E+00 | 1.08E−02 | 4.20E−02 | 5.28E−02 | 2.56E−03 |
| Rectum | 0.00E+00 | 1.08E−02 | 8.35E−02 | 9.43E−02 | 2.17E−03 |
| Heart Wall | 0.00E+00 | 4.78E−03 | 3.42E−02 | 3.90E−02 | 3.60E−04 |
| Kidneys | 0.00E+00 | 1.43E−02 | 4.92E−02 | 6.35E−02 | 5.86E−04 |
| Liver | 0.00E+00 | 4.06E−03 | 3.39E−02 | 3.80E−02 | 1.52E−03 |
| Lungs | 0.00E+00 | 8.84E−04 | 2.96E−02 | 3.05E−02 | 3.66E−03 |
| Ovaries | 0.00E+00 | 1.59E−03 | 5.23E−02 | 5.39E−02 | 2.16E−03 |
| Pancreas | 0.00E+00 | 4.28E−03 | 8.23E−02 | 8.66E−02 | 8.00E−04 |
| Salivary Glands | 0.00E+00 | 4.27E−03 | 1.94E−02 | 2.37E−02 | 2.37E−04 |
| Red Marrow | 0.00E+00 | 3.87E−02 | 2.77E−02 | 3.16E−02 | 3.79E−03 |
| Osteogenic Cells | 0.00E+00 | 4.41E−03 | 4.15E−02 | 4.59E−02 | 4.59E−04 |
| Spleen | 0.00E+00 | 4.29E−03 | 8.02E−02 | 8.45E−02 | 7.80E−04 |
| Thymus | 0.00E+00 | 4.28E−03 | 2.31E−02 | 2.74E−02 | 2.53E−04 |
| Thyroid | 0.00E+00 | 4.27E−03 | 1.96E−02 | 2.38E−02 | 9.53E−04 |
| Urinary Bladder Wall | 0.00E+00 | 1.31E−01 | 1.56E−01 | 2.87E−01 | 1.15E−02 |
| Uterus | 0.00E+00 | 4.27E−03 | 8.52E−02 | 8.95E−02 | 4.13E−04 |
| Total Body | 0.00E+00 | 6.46E−03 | 2.92E−02 | 3.57E−02 | 0.00E+00 |
| Effective Dose | 8.61E−02 | | | | |

TABLE 15
Calculated effective dose from administration
of [¹¹¹In]In-DPI-4452

| | Dose (mSv/MBq) |
|---|---|
| Effective dose to ICRP89 male phantom | 8.68E−02 |
| Effective dose to ICRP89 female phantom | 1.19E−01 |
| Average effective dose | 1.03E−01 |

TABLE 16
Calculated effective dose from administration
of [¹¹¹In]In-DPI-4501

| | Dose (mSv/MBq) |
|---|---|
| Effective Dose to ICRP89 male phantom | 8.27E−02 |
| Effective Dose to ICRP89 female phantom | 8.61E−02 |
| Average Effective dose | 8.44E−02 |

The estimated residence times for the ¹⁷⁷Lu-labelled DPI-4452 and DPI-4501 are presented in Table 17, Table 18, Table 19 and Table 20. Extrapolated Lu-177 radiation doses absorbed in human organs are presented in Table 21 and Table 22, for [¹⁷⁷Lu]Lu-DPI-4452, and in Table 23 and Table 24, for [¹⁷⁷Lu]Lu-DPI-4501. Estimates of the maximum allowed radioactivity dose in every organ according to tolerable limits set for external radiation beam therapy (taken by default as a conservative approach) are presented in Table 25 (for [¹⁷⁷Lu]Lu-DPI-4452) and Table 26 (for [¹⁷⁷Lu]Lu-DPI-4501).

TABLE 17
Input parameters for the male dose
calculation for [¹⁷⁷Lu]Lu-DPI-4452.

| Organ | Mass (g) | Residence time (h) |
|---|---|---|
| Adrenals | 14 | 0.00E+00 |
| Brain | 1450 | 0.00E+00 |
| Esophagus | 40 | 0.00E+00 |
| Eyes | 15 | 0.00E+00 |
| Gallbladder Contents | 58 | 9.76E−02 |
| Left Colon | 75 | 1.95E−02 |
| Small Intestine | 350 | 3.95E+00 |
| Stomach Contents | 250 | 3.06E+00 |
| Right Colon | 150 | 3.90E−01 |
| Rectum | 75 | 1.95E−01 |
| Heart Contents | 510 | 2.43E−02 |
| Heart Wall | 330 | 0.00E+00 |
| Kidneys | 310 | 1.15E+00 |
| Liver | 1800 | 3.03E+00 |
| Lungs | 1200 | 5.38E−01 |
| Pancreas | 140 | 0.00E+00 |
| Prostate | 17 | 0.00E+00 |
| Salivary Glands | 85 | 0.00E+00 |
| Red Marrow | 1170 | 1.59E−01 |
| Cortical Bone | 4400 | 0.00E+00 |
| Trabecular Bone | 1100 | 0.00E+00 |
| Spleen | 150 | 0.00E+00 |
| Testes | 35 | 5.84E−03 |
| Thymus | 25 | 0.00E+00 |
| Thyroid | 20 | 0.00E+00 |
| Urinary Bladder Contents | 211 | 1.13E+00 |
| Total Body | 73000 | 9.00E+01 |

TABLE 18
Extrapolated input parameters for the female
dose calculation for [¹⁷⁷Lu]Lu-DPI-4452

| Organ | Mass (g) | Residence time (h) |
|---|---|---|
| Adrenals | 13 | 0.00E+00 |
| Brain | 1300 | 0.00E+00 |
| Breasts | 50 | 0.00E+00 |
| Esophagus | 35 | 0.00E+00 |
| Eyes | 15 | 0.00E+00 |
| Gallbladder Contents | 48 | 1.37E−01 |
| Left Colon | 80 | 3.35E−01 |
| Small Intestine | 280 | 5.74E+00 |
| Stomach Contents | 230 | 6.63E+00 |
| Right Colon | 160 | 6.69E−01 |
| Rectum | 80 | 3.35E−01 |
| Heart Contents | 370 | 2.44E−02 |
| Heart Wall | 250 | 0.00E+00 |
| Kidneys | 275.5 | 1.15E+00 |
| Liver | 1400 | 3.99E+00 |
| Lungs | 950 | 7.53E−01 |
| Ovaries | 11 | 8.09E−03 |
| Pancreas | 120 | 0.00E+00 |
| Salivary Glands | 70 | 0.00E+00 |
| Red Marrow | 900 | 1.84E−01 |
| Cortical Bone | 3200 | 0.00E+00 |
| Trabecular Bone | 800 | 0.00E+00 |
| Spleen | 130 | 0.00E+00 |
| Thymus | 20 | 0.00E+00 |
| Thyroid | 17 | 0.00E+00 |
| Urinary Bladder Contents | 160 | 1.52E+00 |
| Uterus | 80 | 0.00E+00 |
| Total Body | 60000 | 5.94E+01 |

TABLE 19
Input parameters for the male dose
calculation for [¹⁷⁷Lu]Lu-DPI-4501

| | | Kinetics Value |
|---|---|---|
| Source Organ Name | Mass [g] | [MBq-h/MBq] |
| Adrenals | 14 | 0.00E+00 |
| Brain | 1450 | 0.00E+00 |
| Esophagus | 40 | 0.00E+00 |
| Eyes | 15 | 0.00E+00 |
| Gallbladder Contents | 58 | 3.30E−02 |
| Left Colon | 75 | 1.40E−01 |
| Small Intestine | 350 | 2.10E+00 |
| Stomach Contents | 250 | 7.22E+00 |
| Right Colon | 150 | 2.79E−01 |
| Rectum | 75 | 1.40E−01 |
| Heart Contents | 510 | 3.23E−02 |
| Heart Wall | 330 | 0.00E+00 |
| Kidneys | 310 | 6.12E−01 |
| Liver | 1800 | 1.03E+00 |
| Lungs | 1200 | 8.51E−02 |
| Pancreas | 140 | 0.00E+00 |
| Prostate | 17 | 0.00E+00 |
| Salivary Glands | 85 | 0.00E+00 |
| Red Marrow | 1170 | 5.02E−02 |
| Cortical Bone | 4400 | 0.00E+00 |
| Trabecular Bone | 1100 | 0.00E+00 |
| Spleen | 150 | 0.00E+00 |
| Testes | 35 | 1.97E−03 |
| Thymus | 25 | 0.00E+00 |
| Thyroid | 20 | 0.00E+00 |
| Urinary Bladder Contents | 211 | 1.66E+00 |
| Total Body | 73000 | 3.45E+01 |

TABLE 20
Input parameters for the female dose
calculation for [¹⁷⁷Lu]Lu-DPI-4501.

| | | Kinetics Value |
|---|---|---|
| Source Organ Name | Mass [g] | [MBq-h/MBq] |
| Adrenals | 13 | 0.00E+00 |
| Brain | 1300 | 0.00E+00 |
| Breasts | 500 | 0.00E+00 |
| Esophagus | 35 | 0.00E+00 |
| Eyes | 15 | 0.00E+00 |
| Gallbladder Contents | 48 | 2.17E−02 |
| Left Colon | 80 | 1.07E−01 |
| Small Intestine | 280 | 1.34E+00 |
| Stomach Contents | 230 | 7.19E+00 |
| Right Colon | 160 | 2.14E−01 |
| Rectum | 80 | 1.07E−01 |
| Heart Contents | 370 | 2.31E−02 |
| Heart Wall | 250 | 0.00E+00 |
| Kidneys | 275.5 | 3.95E−01 |
| Liver | 1400 | 6.34E−01 |
| Lungs | 950 | 8.76E−02 |
| Ovaries | 11 | 1.92E−03 |
| Pancreas | 120 | 0.00E+00 |
| Salivary Glands | 70 | 0.00E+00 |
| Red Marrow | 900 | 4.45E−02 |
| Cortical Bone | 3200 | 0.00E+00 |
| Trabecular Bone | 800 | 0.00E+00 |
| Spleen | 130 | 0.00E+00 |
| Thymus | 20 | 0.00E+00 |
| Thyroid | 17 | 0.00E+00 |
| Urinary Bladder Contents | 160 | 2.06E+00 |
| Uterus | 80 | 0.00E+00 |
| Total Body | 60000 | 2.86E+01 |

TABLE 21
Extrapolated calculated OLINDA output data for the human male
ICRP-89 phantom per MBq administered [¹⁷⁷Lu]Lu-DPI-4452.
Values are given in mGy/MBq for Beta, Gamma and Total, whereas
ICRP-103 ED and effective dose is given in mSv/MBq.

| Target Organ | Alpha | Beta | Gamma | Total | ICRP-103 ED |
|---|---|---|---|---|---|
| Adrenals | 0.00E+00 | 1.07E−01 | 1.72E−02 | 1.25E−01 | 1.15E−03 |
| Brain | 0.00E+00 | 1.05E−01 | 8.60E−03 | 1.14E−01 | 1.14E−03 |
| Esophagus | 0.00E+00 | 1.05E−01 | 1.23E−02 | 1.17E−01 | 4.70E−03 |
| Eyes | 0.00E+00 | 1.77E−01 | 8.60E−03 | 1.14E−01 | 0.00E+00 |
| Gallbladder Wall | 0.00E+00 | 2.16E−01 | 1.67E−02 | 1.94E−01 | 1.79E−03 |
| Left colon | 0.00E+00 | 5.86E−01 | 1.90E−02 | 2.35E−01 | 1.14E−02 |
| Small Intestine | 0.00E+00 | 6.28E−01 | 2.29E−02 | 6.09E−01 | 5.62E−03 |
| Stomach Wall | 0.00E+00 | 2.16E−01 | 2.60E−02 | 6.54E−01 | 7.85E−02 |
| Right colon | 0.00E+00 | 2.16E−01 | 1.65E−02 | 2.33E−01 | 1.13E−02 |
| Rectum | 0.00E+00 | 1.07E−01 | 1.64E−02 | 2.33E−01 | 5.35E−03 |
| Heart Wall | 0.00E+00 | 3.16E−01 | 1.19E−02 | 1.19E−01 | 1.10E−03 |
| Kidneys | 0.00E+00 | 1.43E−01 | 1.73E−02 | 3.33E−01 | 3.07E−03 |
| Liver | 0.00E+00 | 3.80E−02 | 1.42E−02 | 1.57E−01 | 6.30E−03 |
| Lungs | 0.00E+00 | 1.05E−01 | 1.03E−02 | 4.83E−02 | 5.80E−03 |
| Pancreas | 0.00E+00 | 1.05E−01 | 2.29E−02 | 1.28E−01 | 1.18E−03 |
| Prostate | 0.00E+00 | 1.05E−01 | 1.52E−02 | 1.20E−01 | 5.56E−04 |
| Salivary Glands | 0.00E+00 | 8.55E−02 | 1.09E−02 | 1.16E−01 | 1.16E−03 |
| Red Marrow | 0.00E+00 | 1.15E−01 | 1.06E−02 | 9.61E−02 | 1.15E−02 |
| Osteogenic Cells | 0.00E+00 | 1.05E−01 | 2.07E−02 | 1.35E−01 | 1.35E−03 |
| Spleen | 0.00E+00 | 1.41E−02 | 1.52E−02 | 1.20E−01 | 1.11E−03 |
| Testes | 0.00E+00 | 3.34E−01 | 8.69E−03 | 2.28E−02 | 9.11E−04 |
| Thymus | 0.00E+00 | 1.05E−01 | 9.92E−03 | 1.15E−01 | 1.06E−03 |
| Thyroid | 0.00E+00 | 1.05E−01 | 1.06E−02 | 1.16E−01 | 4.63E−03 |
| Urinary Bladder Wall | 0.00E+00 | 3.34E−01 | 1.87E−02 | 3.53E−01 | 1.41E−02 |
| Total Body | 0.00E+00 | 1.21E−01 | 1.07E−02 | 1.32E−01 | 0.00E+00 |
| Effective Dose | 1.75E−01 | | | | |

TABLE 22
Extrapolated calculated OLINDA output data for the human female
ICRP-89 phantom per MBq administered [¹⁷⁷Lu]Lu-DPI-4452.
Values are given in mGy/MBq for Beta, Gamma and Total, whereas
ICRP-103 ED and effective dose is given in mSv/MBq.

| Target Organ | Alpha | Beta | Gamma | Total | ICRP-103 ED |
|---|---|---|---|---|---|
| Adrenals | 0.00E+00 | 8.60E−02 | 2.31E−02 | 1.09E−01 | 1.01E−03 |
| Brain | 0.00E+00 | 8.45E−02 | 7.05E−03 | 9.15E−02 | 9.15E−04 |
| Breasts | 0.00E+00 | 8.45E−02 | 6.45E−03 | 9.09E−02 | 1.09E−02 |
| Esophagus | 0.00E+00 | 8.45E−02 | 1.08E−02 | 9.53E−02 | 3.81E−03 |
| Eyes | 0.00E+00 | 8.45E−02 | 7.05E−03 | 9.15E−02 | 0.00E+00 |
| Gallbladder Wall | 0.00E+00 | 2.06E−01 | 2.16E−02 | 2.28E−01 | 2.10E−03 |
| Left colon | 0.00E+00 | 2.63E−01 | 2.02E−02 | 2.83E−01 | 1.37E−02 |
| Small Intestine | 0.00E+00 | 9.59E−01 | 2.79E−02 | 9.87E−01 | 9.11E−03 |
| Stomach Wall | 0.00E+00 | 1.31E+00 | 4.84E−02 | 1.36E−00 | 1.64E−01 |
| Right colon | 0.00E+00 | 2.63E−01 | 1.84E−02 | 2.81E−01 | 1.36E−02 |
| Rectum | 0.00E+00 | 2.63E−01 | 1.73E−02 | 2.80E−01 | 6.44E−03 |
| Heart Wall | 0.00E+00 | 8.74E−02 | 1.22E−02 | 9.96E−02 | 9.19E−04 |
| Kidneys | 0.00E+00 | 3.54E−01 | 2.00E−02 | 3.74E−01 | 3.45E−03 |
| Liver | 0.00E+00 | 2.43E−01 | 1.90E−02 | 2.62E−01 | 1.05E−02 |
| Lungs | 0.00E+00 | 6.72E−02 | 1.15E−02 | 7.87E−02 | 9.44E−03 |
| Ovaries | 0.00E+00 | 6.19E−02 | 1.59E−02 | 7.78E−02 | 3.11E−03 |
| Pancreas | 0.00E+00 | 8.48E−02 | 2.75E−02 | 1.12E−01 | 1.04E−03 |
| Salivary Glands | 0.00E+00 | 8.45E−02 | 7.40E−03 | 9.19E−02 | 9.19E−04 |
| Red Marrow | 0.00E+00 | 7.24E−02 | 1.01E−02 | 8.25E−02 | 9.90E−03 |
| Osteogenic Cells | 0.00E+00 | 6.97E−02 | 1.79E−02 | 8.76E−02 | 8.76E−04 |
| Spleen | 0.00E+00 | 8.48E−02 | 2.27E−02 | 1.07E−01 | 9.92E−04 |
| Thymus | 0.00E+00 | 8.48E−02 | 9.33E−03 | 9.41E−02 | 8.69E−04 |
| Thyroid | 0.00E+00 | 8.45E−02 | 7.58E−03 | 9.21E−02 | 3.68E−03 |
| Urinary Bladder Wall | 0.00E+00 | 4.91E−01 | 1.90E−02 | 5.10E−01 | 2.04E−02 |
| Uterus | 0.00E+00 | 8.45E−02 | 1.90E−02 | 1.04E−01 | 4.78E−04 |
| Total Body | 0.00E+00 | 1.15E−01 | 1.05E−02 | 1.26E−01 | 0.00E+00 |
| Effective Dose | 2.92E−01 | | | | |

TABLE 23
Extrapolated calculated effective dose from
administration of [¹⁷⁷Lu]Lu-DPI-4452

| | Dose (mSv/MBq) |
|---|---|
| Effective Dose to ICRP89 male phantom | 1.75E−01 |
| Effective Dose to ICRP89 female phantom | 2.92E−01 |
| Average Effective dose | 2.34E−01 |

TABLE 24
Extrapolated calculated OLINDA output data for the human male
ICRP-89 phantom per MBq administered [¹⁷⁷Lu]Lu-DPI-4501.
Values are given in mGy/MBq for Beta, Gamma and Total, whereas
ICRP-103 ED and effective dose is given in mSv/MBq.

| Target Organ | Alpha | Beta | Gamma | Total | ICRP-103 ED |
|---|---|---|---|---|---|
| Adrenals | 0.00E+00 | 4.13E−02 | 1.08E−02 | 5.20E−02 | 4.80E−04 |
| Brain | 0.00E+00 | 4.03E−02 | 3.31E−03 | 4.36E−02 | 4.36E−04 |
| Esophagus | 0.00E+00 | 4.03E−02 | 9.27E−03 | 4.96E−02 | 1.98E−03 |
| Eyes | 0.00E+00 | 4.03E−02 | 3.32E−03 | 4.36E−02 | 0.00E+00 |
| Gallbladder Wall | 0.00E+00 | 6.48E−02 | 7.83E−03 | 7.26E−02 | 6.70E−04 |
| Left colon | 0.00E+00 | 1.20E−01 | 1.19E−02 | 1.32E−01 | 6.38E−03 |
| Small Intestine | 0.00E+00 | 2.96E−01 | 1.21E−02 | 3.08E−01 | 2.85E−03 |
| Stomach Wall | 0.00E+00 | 1.27E+00 | 4.05E−02 | 1.31E+00 | 1.58E−01 |
| Right Colon | 0.00E+00 | 1.20E−01 | 7.73E−03 | 1.27E−01 | 6.18E−03 |
| Rectum | 0.00E+00 | 1.20E−01 | 8.29E−03 | 1.28E−01 | 2.94E−03 |
| Heart Wall | 0.00E+00 | 4.30E−02 | 8.92E−03 | 5.19E−02 | 4.79E−04 |
| Kidneys | 0.00E+00 | 1.68E−01 | 9.12E−03 | 1.77E−01 | 1.63E−03 |
| Liver | 0.00E+00 | 4.85E−02 | 7.17E−03 | 5.56E−02 | 2.23E−03 |
| Lungs | 0.00E+00 | 6.04E−03 | 6.28E−03 | 1.23E−02 | 1.48E−03 |
| Pancreas | 0.00E+00 | 4.03E−02 | 2.29E−02 | 6.32E−02 | 5.83E−04 |
| Prostate | 0.00E+00 | 4.03E−02 | 7.83E−03 | 4.81E−02 | 2.22E−04 |
| Salivary Glands | 0.00E+00 | 4.03E−02 | 4.23E−03 | 4.45E−02 | 4.45E−04 |
| Red Marrow | 0.00E+00 | 3.23E−02 | 5.04E−03 | 3.74E−02 | 4.48E−03 |
| Osteogenic Cells | 0.00E+00 | 4.37E−02 | 9.04E−03 | 5.27E−02 | 5.27E−04 |
| Spleen | 0.00E+00 | 4.04E−02 | 1.16E−02 | 5.19E−02 | 4.79E−04 |
| Testes | 0.00E+00 | 4.75E−03 | 3.69E−03 | 8.44E−03 | 3.38E−04 |
| Thymus | 0.00E+00 | 4.03E−02 | 4.70E−03 | 4.50E−02 | 4.15E−04 |
| Thyroid | 0.00E+00 | 4.03E−02 | 4.38E−03 | 4.47E−02 | 1.79E−03 |
| Urinary Bladder Wall | 0.00E+00 | 3.76E−01 | 1.47E−02 | 3.90E−01 | 1.56E−02 |
| Total Body | 0.00E+00 | 5.59E−02 | 4.69E−03 | 6.06E−02 | 0.00E+00 |
| Effective Dose | 2.10E−01 | | | | |

TABLE 25
Extrapolated calculated OLINDA output data for the human female
ICRP-89 phantom per MBq administered[¹⁷⁷Lu]Lu-DPI-4501.
Values are given in mGy/MBq for Beta, Gamma and Total, whereas
ICRP-103 ED and effective dose is given in mSv/MBq.

| Target Organ | Alpha | Beta | Gamma | Total | ICRP-103 ED |
|---|---|---|---|---|---|
| Adrenals | 0.00E+00 | 4.11E−02 | 1.35E−02 | 5.46E−02 | 5.04E−04 |
| Brain | 0.00E+00 | 4.07E−02 | 3.39E−03 | 4.41E−02 | 4.41E−04 |
| Breasts | 0.00E+00 | 4.07E−02 | 3.28E−03 | 4.40E−02 | 5.27E−03 |
| Esophagus | 0.00E+00 | 4.07E−02 | 5.84E−03 | 4.65E−02 | 1.86E−03 |
| Eyes | 0.00E+00 | 4.07E−02 | 3.39E−03 | 4.41E−02 | 0.00E+00 |
| Gallbladder Wall | 0.00E+00 | 6.00E−02 | 8.13E−03 | 6.81E−02 | 6.29E−04 |
| Left colon | 0.00E+00 | 9.77E−02 | 1.08E−02 | 1.09E−01 | 5.26E−03 |
| Small Intestine | 0.00E+00 | 2.44E−01 | 1.14E−02 | 2.55E−01 | 2.36E−03 |
| Stomach Wall | 0.00E+00 | 1.37E+00 | 4.44E−02 | 1.42E+00 | 1.70E−01 |
| Right Colon | 0.00E+00 | 9.77E−02 | 7.30E−03 | 1.05E−01 | 5.09E−03 |
| Rectum | 0.00E+00 | 9.77E−02 | 1.03E−02 | 1.08E−01 | 2.48E−03 |
| Heart Wall | 0.00E+00 | 4.33E−02 | 7.04E−03 | 5.04E−02 | 4.65E−04 |
| Kidneys | 0.00E+00 | 1.22E−01 | 8.86E−03 | 1.30E−01 | 1.20E−03 |
| Liver | 0.00E+00 | 3.85E−02 | 6.62E−03 | 4.51E−02 | 1.81E−03 |
| Lungs | 0.00E+00 | 7.83E−03 | 5.98E−03 | 1.38E−02 | 1.66E−03 |
| Ovaries | 0.00E+00 | 1.47E−02 | 7.94E−03 | 2.26E−02 | 9.05E−04 |
| Pancreas | 0.00E+00 | 4.07E−02 | 1.68E−02 | 5.75E−02 | 5.31E−04 |
| Salivary Glands | 0.00E+00 | 4.07E−02 | 3.57E−03 | 4.42E−02 | 4.42E−04 |
| Red Marrow | 0.00E+00 | 3.25E−02 | 5.02E−03 | 3.75E−02 | 4.50E−03 |
| Osteogenic Cells | 0.00E+00 | 3.27E−02 | 8.82E−03 | 4.15E−02 | 4.15E−04 |
| Spleen | 0.00E+00 | 4.08E−02 | 1.65E−02 | 5.72E−02 | 5.28E−04 |
| Thymus | 0.00E+00 | 4.07E−02 | 4.40E−03 | 4.51E−02 | 4.16E−04 |
| Thyroid | 0.00E+00 | 4.07E−02 | 3.61E−03 | 4.43E−02 | 1.77E−03 |
| Urinary Bladder Wall | 0.00E+00 | 5.89E−01 | 1.49E−02 | 6.04E−01 | 2.42E−02 |
| Uterus | 0.00E+00 | 4.07E−02 | 1.09E−02 | 5.15E−02 | 2.38E−04 |
| Total Body | 0.00E+00 | 5.80E−02 | 5.27E−03 | 6.33E−02 | 0.00E+00 |
| Effective Dose | 2.33E−01 | | | | |

TABLE 26
Extrapolated calculated effective dose from
administration of [¹⁷⁷Lu]Lu-DPI-4501

| | Dose (mSv/MBq) |
|---|---|
| Effective Dose to ICRP89 male phantom | 2.10E−01 |
| Effective Dose to ICRP89 female phantom | 2.33E−01 |
| Average Effective dose | 2.22E−01 |

TABLE 27
Estimate of the maximum allowed injected radioactivity dose
of [¹⁷⁷Lu]Lu-DPI-4452, based on individual organ dose
limits and the calculated absorbed radiation dose per injection/MBq.
The limiting organ is written in bold. Note that allowed
injected radioactivity dose refers to the maximum administered
radioactivity enabling the radiation dose absorbed in an
organ to remain below the given dose limit.

| | Absorbed | Absorbed | Maximum allowed |
|---|---|---|---|
| | radiation dose per | dose | injected |
| | injected activity | limit | radioactivity |
| Organ | (mGy/MBq) | (Gy)* | dose (MBq) |
| Left colon | 2.35E−01 | 25 | 1.06E+05 |
| Small Intestine | 6.09E−01 | 18 | 2.96E+04 |
| Stomach Wall | 6.54E−01 | 28 | 4.28E+04 |
| Right Colon | 2.33E−01 | 25 | 1.07E+05 |
| Rectum | 2.33E−01 | 25 | 1.07E+05 |
| Heart Wall | 1.19E−01 | 32 | 2.69E+05 |
| Kidneys | 3.33E−01 | 23 | 6.91E+04 |
| Liver | 1.57E−01 | 21 | 1.34E+05 |
| Lungs | 4.83E−02 | 12 | 2.48E+05 |
| Urinary | 3.53E−01 | 18.3 | 5.18E+04 |
| Bladder Wall | | | |

*Emami B. 2013. ‘Tolerance of Normal Tissue to Therapeutic Radiation’, Rep Radiother Oncol, 1 (1): 35-48.

TABLE 28
Estimate of the maximum allowed injected radioactivity dose
of [¹⁷⁷Lu]Lu-DPI-4501, based on individual organ dose
limits and the calculated absorbed radiation dose/per injection/MBq.
The limiting organ is written in bold. Note that allowed
injected radioactivity dose refers to the maximum administered
radioactivity enabling the radiation dose absorbed in an
organ to remain below the given dose limit.

| | Absorbed | Absorbed | Maximum allowed |
|---|---|---|---|
| | radiation dose per | dose | injected |
| | injected activity | limit | radioactivity |
| Organ | (mGy/MBq) | (Gy)* | dose (MBq) |
| Left colon | 1.32E−01 | 25 | 1.89E+05 |
| Small Intestine | 3.08E−01 | 18 | 5.84E+04 |
| Stomach Wall | 1.31E+00 | 28 | 2.14E+04 |
| Right Colon | 1.27E−01 | 25 | 1.97E+05 |
| Rectum | 1.28E−01 | 25 | 1.95E+05 |
| Heart Wall | 5.19E−02 | 32 | 6.17E+05 |
| Kidneys | 1.77E−01 | 23 | 1.30E+05 |
| Liver | 5.56E−02 | 21 | 3.78E+05 |
| Lungs | 1.23E−02 | 12 | 9.76E+05 |
| Urinary | 3.90E−01 | 18.3 | 4.69E+04 |
| Bladder Wall | | | |

*Emami B. 2013. ‘Tolerance of Normal Tissue to Therapeutic Radiation’, Rep Radiother Oncol, 1 (1): 35-48.

The above extrapolations were made under the assumption that CAIX expression levels in humans are similar to dogs.

For [¹⁷⁷Lu]Lu-DPI-4452, the dose-limiting organ appeared to be the small intestine, and the maximum allowed radioactivity starting dose would be 29.6 GBq. For this administered radioactivity dose, the estimated radiation dose delivered to a representative 11.0-g tumor is within the range 12.2-660 Gy (Table 29), which is compatible with antitumoral effect in humans.

TABLE 29
Tumor radiation dose at maximum injected
activity for [¹⁷⁷Lu]Lu-DPI-4452.

| | Dose at Max | Dose at Max |
|---|---|---|
| | tolerated activity | tolerated activity |
| Tumor mass (g) | % ID method (Gy) | % ID/g method (Gy) |
| 3.00E+00 | 2.40E+03 | 1.22E+01 |
| 1.10E+01 | 6.60E+02 | 1.22E+01 |
| 3.00E+01 | 2.43E+02 | 1.23E+01 |
| 1.00E+02 | 7.34E+01 | 1.24E+01 |
| 5.94E+02 | 1.26E+01 | 1.26E+01 |

For [¹⁷⁷Lu]Lu-DPI-4501, the dose-limiting organ appeared to be the stomach wall, and the maximum allowed radioactivity starting dose would be 21.4 GBq. For this administered radioactivity dose, the estimated radiation dose delivered to a representative 11.0-g tumor is within the range 4.4-205 Gy (Table 30).

TABLE 30
Tumor radiation dose at maximum injected
activity for [¹⁷⁷Lu]Lu-DPI-4501.

| | Dose at Max | Dose at Max |
|---|---|---|
| | tolerated activity | tolerated activity |
| Tumor mass (g) | % ID method (Gy) | % ID/g method (Gy) |
| 3.00E+00 | 7.46E+02 | 4.32E+00 |
| 1.10E+01 | 2.05E+02 | 4.36E+00 |
| 3.00E+01 | 7.55E+01 | 4.36E+00 |
| 1.00E+02 | 2.29E+01 | 4.40E+00 |
| 5.94E+02 | 4.47E+00 | 4.47E+00 |

### Example 7: Binding Study to Human, Dog, and Mouse CAIX

The species cross-reactivity of DPI-4452 and DPI-4501 for CAIX was investigated by measuring the equilibrium dissociation constant Kd in CHO cells transfected with human, dog or mouse CAIX in a radioligand binding assay, employing the ¹¹¹In-labeled versions of DPI-4452 and DPI-4501 at 8 different concentrations. After attainment of equilibrium, the cells were harvested, and the bound fraction of the compounds was measured. The resulting saturation binding data were analyzed using Graph Pad Prism 8.3.

CHO cells transfected with human, dog, and mouse CAIX (CHO-huCA9 T04J-1/20 K1, CHO-dogCA9 T05J-9/20 K4, CHO-murCA9 T05J-3/20 K4) were obtained from InSCREENex (Germany).

For radiolabeling, 200 μM stock solutions of DPI-4452 and DPI-4501 were prepared by dissolution in 0.1 M HEPES, aliquoted and stored at −20° C. A molar excess of reference compound AcVY-[C(3MeBn)-EPDWLTWSC]-NH2 was used as a blocking peptide to assess non-specific binding in autoradiographic studies. The reference compound binds with a similar affinity to CAIX and blocks binding sites of test compounds. For the blocking solution, a 10 mM stock solution of the reference compound was prepared by dissolution in DMSO.

CHO cells were maintained in Ham's medium supplemented with 10% fetal bovine serum, 2 mM L-glutamine, 100 U/mL Penicillin and 0.1 mg/mL streptomycin under standard cell culture conditions. The cells were grown in uncoated cell culture flasks (150 cm², Biochrom) to subconfluence and then split 1:2-1:3. Approximately 24 hours before the assay, cells were detached by incubation with Accutase and carefully tapping the flasks. Detached cells were resuspended in medium and collected by centrifugation (300 g, 5 min, RT). Cell pellets were resuspended in cell culture medium and counted using a particle counter (CASY Model TT, Scharfe Systems, Germany). Cell concentrations were adjusted to 3×10⁵ mL⁻¹, and 1.000 μL per well of the suspension were dispensed into poly-D-lysine-coated flat-clear-bottom 24 well plates.

Compound radiolabeling was performed as follows. At the start of synthesis, volumes of radionuclide solution (¹¹¹InCl₃ in 20 mM HCl) containing required amounts of activity were mixed with appropriate volumes of 200 μM DPI-4452 and DPI-4501 stock solutions to yield a specific activity of 45 MBq/nmol. Then, 25 mg/mL methionine in 1 M sodium acetate buffer pH 5 was added at a final concentration of 0.1 M sodium acetate. Subsequently, the mixture was heated to 80° C. for 25 min, followed by cooling for 5 minutes. Finally, 20 μL of 200 mg/mL ascorbic acid solution, 2.5 μL of 5 mg/mL DTPA and 2.5 μL of 5% TWEEN-20 per 100 μL reaction mixture were added, resulting in the concentrated study solution. For quality control, an aliquot of the labeling solution was diluted 1:40 with 0.1% TWEEN-20 in 0.1 M sodium acetate buffer pH 5. Five microliters of the diluted labeling solution were injected onto a Poroshell SB-C18 2.1×50 mm, 2.7 μm column. HPLC analysis was performed as follows: Gradient A: H₂O, 0.1% TFA, Gradient B: Acetonitrile (MeCN), gradient from 5% B to 70% B within 15 min, flow rate of 0.5 mL/min; detector: NaI, DAD 215 nm. The peak eluting with the dead volume represents free radionuclide, the peak eluting with the ligand-specific retention time as determined with a non-labeled sample represents the radiolabeled compound. The radiochemical purities (RCP's) of [¹¹¹In]In-DPI-4452 and [¹¹¹In]In-DPI-4501 according to HPLC were 88% and ≥86%, respectively.

The assessment of the time needed until attainment of equilibration on CHO-huCAIX and CHO-dgCAIX was performed as follows. The 10 mM reference compound stock solution was diluted with assay medium (Ham's medium without additives) to prepare an 8-μM blocking working solution. The radiolabeling mixtures were diluted with assay medium to prepare 160 nM radioligand working solutions. Subsequently, 1.6 and 3.2 nM radioligand dilutions were prepared by diluting the radioligand working solutions 1:100 and 1:50, respectively, with assay medium. Approximately 24 hours after re-seeding (3×10⁵ mL⁻¹ cells per well), the medium was aspirated, and the cells were washed once with assay medium (700 μL). To determine total binding, 700 μL of binding medium and 100 μL of the radioligand dilutions were added to the wells in triplicates.

To determine non-specific binding, 600 μL of binding medium, 100 μL of the 8 μM reference compound blocking solution and 100 μL of the radioligand dilutions were added to the wells in triplicates. The plates were incubated for 1 h, 3 h, 6 h, and 8 h at 37° C. under standard cell culture conditions (5% CO2). At the end of the incubation time, the plates were placed on ice while aspirating the radioligand solutions. Subsequently, the cells were washed with ice-cold PBS (0.5 mL, 1 mL, 1 mL). 300 μL of RIPA buffer containing PIC was added to each well and the plates were placed on a shaker for 10 min at ambient temperature. 200 μL of the cell lysate of each well was transferred to gamma-counting tubes (12×75 mm; e.g., VWR 212-1809 with caps 217-7004). Their associated radioactivity was counted using a gamma counter. An aliquot of each radioligand dilution was included in the gamma counter measurements to allow for determination of their actual concentrations.

The radioligand saturation binding on CHO-huCAIX and dgCAIX was determined as follows. The 10 mM reference compound stock solution was diluted with assay medium (Ham's medium without additives) to prepare a 5 μM blocking working solution. The radiolabeling mixtures were diluted with assay medium to prepare 160 nM radioligand working solutions. Subsequently, following radioligand dilutions were prepared by diluting the radioligand working solutions with assay medium: i) 80 nM radioligand solution ii) 40 nM radioligand solution iii) 20 nM radioligand solution iv) 10 nM radioligand solution v) 5.0 nM radioligand solution vi) 2.5 nM radioligand solution vii) 1.3 nM radioligand solution Approximately 24 hours after re-seeding (3×10⁵ mL¹ cells per well), the medium was aspirated, and the cells were washed once with assay medium (1 mL). To determine total binding, 700 μL of binding medium and 100 μL of the radioligand dilutions were added to the wells in triplicates. To determine non-specific binding, 600 μL of binding medium, 100 μL of the 5 μM reference compound blocking solution and 100 μL of the radioligand dilutions were added to the wells in triplicates to determine their non-specific binding to the cells. The plates were incubated for 8 h at 37° C. under standard cell culture conditions (5% CO2). At the end of the incubation time the plates were placed on ice while aspirating the radioligand solutions. Aliquots of the 20 nM supernatants were transferred to HPLC vials for analysis of radioligand stability over the incubation time. Subsequently, the cells were washed with ice-cold PBS (0.5 mL, 1 mL, 1 mL). 300 μL of RIPA buffer containing PIC was added to each well and the plates were placed on a shaker for 10 min at ambient temperature. 200 μL of the cell lysate of each well was transferred to gamma-counting tubes. Their associated radioactivity was counted using a gamma counter and normalized to the measured protein concentration of each well (see chapter 6.3.7 BCA protein assay). An aliquot of each radioligand dilution was included in the gamma counter measurements to allow for determination of the actual radioligand concentrations in the dilution series.

The radioligand saturation binding on CHO-msCAIX was determined as follows. The 10 mM reference compound stock solution was diluted with assay medium (Ham's medium without additives) to prepare a 5 μM blocking working solution. The radiolabeling mixtures were diluted with assay medium to prepare 500 nM radioligand working solutions. Subsequently, following radioligand dilutions were prepared by diluting the radioligand working solutions with assay medium: i) 250 nM radioligand solution ii) 125 nM radioligand solution iii) 63 nM radioligand solution iv) 31 nM radioligand solution v) 16 nM radioligand solution vi) 7.8 nM radioligand solution vii) 3.9 nM radioligand solution Approximately 24 hours after re-seeding (3×10⁵ mL⁻¹ cells per well), the medium was aspirated, and the cells were washed once with assay medium (1 mL). To determine total binding, 700 μL of binding medium and 100 μL of the radioligand dilutions were added to the wells in triplicates. To determine non-specific binding, 600 μL of binding medium, 100 μL of the 5 μM reference compound blocking solution and 100 μL of the radioligand dilutions were added to the wells in triplicates to determine their non-specific binding to the cells. The plates were incubated for 8 h at 37° C. under standard cell culture conditions (5% CO2). At the end of the incubation time, the plates were placed on ice while aspirating the radioligand solutions. Subsequently, the cells were washed with ice-cold PBS (0.5 mL, 1 mL, 1 mL). 300 μL of RIPA buffer containing PIC was added to each well and the plates were placed on a shaker for 10 min at ambient temperature. 200 μL of the cell lysate of each well was transferred to gamma-counting tubes. Their associated radioactivity was counted using a gamma counter and normalized to the measured protein concentration of each well. An aliquot of each radioligand dilution was included in the gamma counter measurements to allow for determination of the actual radioligand concentrations in the dilution series.

The protein concentration per well was determined via the BCA protein assay. To this end, 10 μL of each cell lysate was transferred to a 96-well microplate in duplicates before 200 μL of BCA working solution per well was added (microplate procedure according to manufacturer's instruction) and the plate was placed on a plate shaker for 30 seconds. Subsequently, the plate was incubated at 37° C. for 30 min. After cooling to ambient temperature, the absorbance at 562 nm was measured on a plate reader to determine the total protein content of each sample.

Data were analyzed using GraphPad Prism 8.3. The actual radioligand concentrations in the radioligand dilutions were calculated according to following equation:

$$c = Radioactivity concentration ( cpm / μ L ) / Specific activity ( cpm / fmol )$$

For the determination of the equilibration time, the model: Association kinetics—two or more conc. of hot. (Association kinetics at two or more concentrations of radioligand) was used that yielded the corresponding association (kₒₙ) and dissociation (k_{off}) rate constants. The equilibration time (t_{eq}) was then calculated using the following equation (Hulme et al. Br. J. Pharmacol. 2010, 161, 1219-1237):

$${t}_{eq} = 5 \times ln 2 / {k}_{off}$$

For the determination of the equilibrium dissociation constant (Kd) and the concentration of specific binding sites (Bₘₐₓ), the model: One site— Total, accounting for ligand depletion was used. The Kd provided in nM was converted into pKd (negative log of the Kd [M]). The Bₘₐₓ value in cpm was converted into fmol/μg protein using the following equation:

$${B}_{max} ( fmol / μ g prot ) = {B}_{max} ( cpm ) / \{ specific activity ( cpm / fmol ) \times protein content ( μ g prot ) \}$$

### Results

The stability of both test compounds during the radioligand binding assay was confirmed by HPLC analysis. To this end, aliquots of the supernatant were removed for quality control at the end of the incubation time following assay procedure.

After measurement of the dissociation rate constant k_{off} and equilibration time t_{eq} (Table 31), the incubation time for the determination of the equilibrium dissociation constant (Kd) on human, dog, and murine CAIX of [¹¹¹In]In-DPI-4452 and [¹¹¹In]In-DPI-4501 was set to be 8 h.

TABLE 31
Dissociation rate constants and calculated equilibration times

| | CHO-huCAIX | | CHO-dgCAIX | |
|---|---|---|---|---|
| | k_{off} (min⁻¹) | t_{eq} (h) | k_{off} (min⁻¹) | t_{eq} (h) |
| [¹¹¹In]In-DPI-4452 | 5.9 × 10⁻³ | 9.8 | 5.89 × 10⁻³ | 9.9 |
| [¹¹¹In]In-DPI-4501 | 7.2 × 10⁻³ | 8.0 | 9.6 × 10⁻³ | 6.0 |

For the determination of the equilibrium dissociation constants (Kd), the total as well as the non-specifically bound fraction of the radioligand of interest to CHO cells expressing CAIX from different species was plotted against the initial radioligand concentration. Due to [¹¹¹In]In-DPI-4501 data not showing the expected saturation binding pattern at the two highest concentrations (10 nM and 20 nM), only the lower six concentrations (0.16-5.0 nM) were used for CHO-huCAIX and CHO-dgCAIX data analysis of both test compounds. Since, The One site— Total model in GraphPad Prism, accounting for ligand depletion, was used for the analysis of all saturation binding curves since radioligand depletion in CHO-huCAIX and CHO-dgCAIX was significant (up to 64%) and provided the equilibrium dissociation constant (Kd) as well as the concentration of specific binding sites (Bₘₐₓ). Binding of the test compounds to CHO-msCAIX was low and not, or only partially blockable.

Table 32 and Table 33 summarize the equilibrium dissociation constants (pKd) as well as the concentration of specific binding sites (Bₘₐₓ) on CHO cells expressing human, dog and mouse CAIX for compounds [¹¹¹In]In-DPI-4452 and [¹¹¹In]In-DPI-4501. Two independent experiments were carried out with CHO-huCAIX und CHO-dgCAIX, and a single experiment with CHO-msCAIX.

TABLE 32
pK_{d} and Bₘₐₓ values of [¹¹¹In]In-DPI-4452

| | CHO- | CHO- | CHO- |
|---|---|---|---|
| Cell line | huCAIX | dgCAIX | msCAIX |
| i) pKd | 9.7 | 9.7 | 7.2 |
| ii) pKd | 9.3 | 9.5 | n.d. |
| Mean pKd ± SD | 9.5 ± 0.2 | 9.6 ± 0.1 | 7.2 |
| i) Bₘₐₓ [fmol/μg prot] | 12.3 | 9.0 | 5.6 |
| ii) Bₘₐₓ [fmol/μg prot] | 13.3 | 9.6 | n.d. |
| Mean Bₘₐₓ [fmol/μg prot] | 12.8 ± 0.5 | 9.3 ± 0.3 | 5.6 |

TABLE 33
pK_{d} and Bₘₐₓvalues of [¹¹¹In]In-DPI-4501

| | CHO- | CHO- | CHO- |
|---|---|---|---|
| Cell line | huCAIX | dgCAIX | msCAIX |
| iii) pKd | 9.8 | 9.6 | 7.9 |
| iv) pKd | 9.4 | 9.3 | n.d. |
| Mean pKd ± SD | 9.6 ± 0.2 | 9.4 ± 0.1 | 7.9 |
| iii) Bₘₐₓ [fmol/μg prot] | 6.4 | 3.3 | 2.1 |
| iv) Bₘₐₓ [fmol/μg prot] | 8.8 | 6.1 | n.d. |
| Mean Bₘₐₓ [fmol/μg prot] | 7.6 ± 1.2 | 4.7 ± 1.4 | 2.1 |

### Conclusion

Compounds [¹¹¹In]In-DPI-4452 and [¹¹¹In]In-DPI-4501 were found to be potent binders on human and dog CAIX expressed in CHO cells, displaying subnanomolar dissociation constants (pKd >9). For both compounds, the binding affinities for human and dog CAIX were comparable, qualifying dog as a suitable species for non-clinical toxicology studies, in which side effects mediated by specific and non-specific binding of a development candidate are to be assessed. In contrast, the dissociation constants with murine CAIX were approximately two magnitudes higher compared to human CAIX, disqualifying mouse as a suitable species for non-clinical toxicology studies. Moreover, binding of the test compounds to CHO-msCAIX was largely non-specific as evidenced by the nominal degree of blocking achievable via addition of an excess of non-labeled compound.

### Example 8: Dose Range Finding Study by Intravenous Route

DPI-4452 was administered by intravenous (i.v.) bolus injection to male beagle dogs at ascending dose levels of 25, 80, 400, and 800 μg/kg/day to one group of two dogs as one single-dose followed by 3 days of wash-out period.

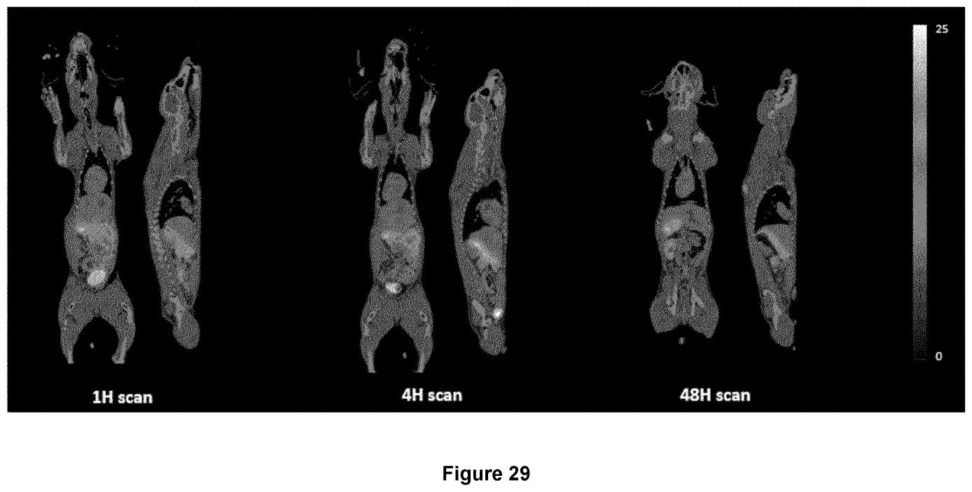

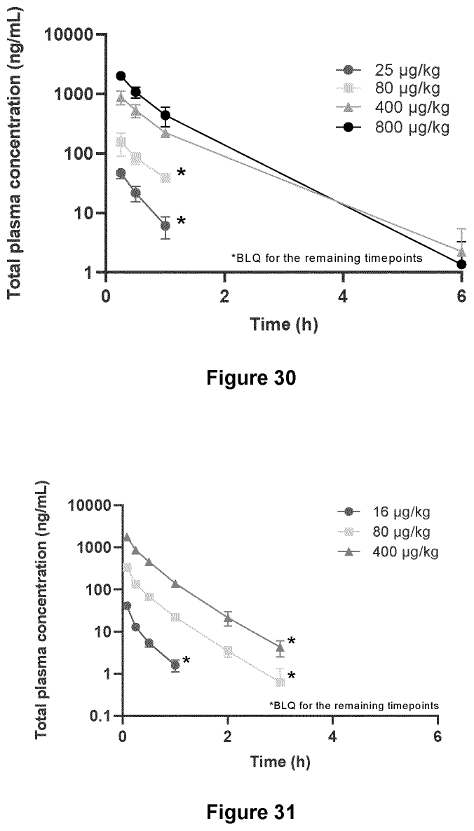

The following parameters and endpoints were evaluated: mortality, clinical observations, body weights, food consumption, local reactions, clinical pathology parameters (hematology, coagulation, and clinical chemistry), organ weights, and macroscopic examinations. Both animals were sampled for toxicokinetics (TK) on each dosing occasion (i.e., on day 1, day 5, day 9 and day 13) at: pre-dose, 15 min, 30 min, 1 h, 6 h and 24 h. Blood samples were collected in K₂EDTA tubes. Plasma was prepared by centrifugation (2500 g for 10 minutes, +4° C.) within 1 hour after collection, and then frozen within 1 hour after centrifugation and stored at −80° C. The quantification of DPI-4452 concentration in the dog plasma samples was performed using DPI-4501, an analog compound, as internal standard, and using solid-phase extraction followed by liquid chromatography—high-resolution mass spectrometry (LC-HRMS) analysis (quantification range of 2.00 ng/mL to 1000 ng/mL). Chromatographic separation was achieved using a Waters Acquity UPLC system with a Waters Acquity HSS T3 C18 2.1×50 mm, 1.8 μm column. Chromatography was conducted at 50° C. at a flow rate of 0.7 mL/min using a mobile phase consisting of A: acetonitrile, and B: 1% formic acid in water, according to the following linear gradient: 0 to 0.1 min: 90% B; 0.1 to 4.1 min: from 90% B to 74% B; 4.1 to 4.2 min: from 74% B to 5% B; 4.2 to 4.6 min: 5% B; 4.6 to 4.7 min: from 5% B to 90% B; 4.7-5.1 min; 90% B. Detection was performed using a Sciex AP16600 TOF mass spectrometer. Toxicokinetic parameters were estimated from concentration-time data using Phoenix pharmacokinetic software (version 6.4, Certara L.P.). A non-compartmental approach consistent with the bolus intravenous injection was used for parameter estimation. Administrations up to 800 μg/kg were well tolerated systemically and locally. Neither in-life parameters nor clinical pathology parameters were affected. In addition, there were no related macroscopic observations noted at necropsy. The TK parameters are presented in Table 34. The data suggest that exposure increased more than dose-proportionally (FIG. 30). In conclusion, doses up to 800 μg/kg/day were tolerated.

TABLE 34
Mean plasma toxicocokinetic parameters of DPI-4452 in the dog (n = 2)

| | Dose | | Dose- | | | | | |
|---|---|---|---|---|---|---|---|---|
| | level | AUCₗₐₛₜ | normalized | C_{15 min} | tₗₐₛₜ | CL | Vₛₛ | t_{1/2} |
| Period | (mg/kg) | (h × ng/mL) | AUCₗₐₛₜ | (ng/mL) | (h) | (mL/min/kg) | (L/kg) | (h) |
| Day 1 | 0.025 | 34.0 | 1.36 | 46.8 | 1; 1 | 11.7 | 0.22 | 0.25 |
| Day 5 | 0.08 | 117 | 1.46 | 156 | 1; 1 | n.a. | n.a. | n.a. |
| Day 9 | 0.4 | 926 | 2.31 | 880 | 1; 6 | 6.83 | 0.24 | 0.49 |
| Day 13 | 0.8 | 3530 | 4.42 | 2000 | 3.2; 6 | 3.52 | 0.32 | 0.88 |

AUCₗₐₛₜ = area under the plasma concentration-time curve until the last sample, Dose-normalized AUCₗₐₛₜ = reported AUCₗₐₛₜ divided by the dose level in μg/kg, CL = clearance, C_{15 min} = measured concentration at 15 min post injection, n.a. = not applicable, t_{1/2} = half-life, tₗₐₛₜ = time to last measurable concentration, Vss = volume of distribution at steady state.

### Example 9: Extended Single-Dose Toxicity Study Including Safety Pharmacology Endpoints by Intravenous Bolus Administration

In this GLP-compliant study, DPI-4452 was administered in an extended single i.v. dose in beagle dogs, including safety pharmacology endpoints at 16, 80, or 400 μg/kg in 2 subsets as described in Table 35.

TABLE 35
Experimental design of the extended single-
dose toxicity study in beagle dogs

| | | | Number and Sex of the Animals | |
|---|---|---|---|---|
| | | | Subset A: FOB and | Subset B: External |
| | | Dose | Toxicokinetics | Telemetry |
| Group | Test | Level | Early Terminal | Late Terminal |
| Number | Material | (μg/kg) | Sacrifice, Day 2 | Sacrifice, Day 16 |
| 1 | Control | 0 | 3M + 3F | 2M + 2F |
| | item | | | |
| 2 | DPI-4452 | 16 | 3M + 3F | 2M + 2F |
| 3 | DPI-4452 | 80 | 3M + 3F | 2M + 2F |
| 4 | DPI-4452 | 400 | 3M + 3F | 2M + 2F |

M = male, F = female, FOB = Functional Observational Battery

The following parameters and endpoints were evaluated: mortality, clinical observations, body weights, food consumption, body temperature, local reactions, ophthalmology, clinical pathology parameters (hematology, coagulation, clinical chemistry, and urinalysis), cardiovascular and respiratory safety pharmacology endpoints along with FOB evaluation, organ weights, and macroscopic and microscopic examinations, and TK parameters. The animals from subset A (3 males and 3 females per group) were sampled for toxicokinetics (TK) at: pre-dose, 5 min, 15 min, 30 min, 1 h, 2 h, 3 h and 6 h. Blood samples were collected in K₂EDTA tubes. Plasma was prepared by centrifugation (2500 g for 10 minutes, +4° C.) within 1 hour after collection, and then frozen within 1 hour after centrifugation and stored at −80° C. The quantification of DPI-4452 concentration in the dog plasma samples was performed using (¹³C6-¹⁵N)-DPI-4452 as internal standard and using solid-phase extraction followed by liquid chromatography-high-resolution mass spectrometry (LC-HRMS) analysis (lower limit of quantification: 1 ng/mL). Chromatographic separation was achieved using a Waters Acquity UPLC system with a Waters Acquity HSS T3 C18 2.1×50 mm, 1.8 μm column. Chromatography was conducted at 50° C. at a flow rate of 0.7 mL/min using a mobile phase consisting of A: 1% formic acid in acetonitrile, and B: 1% formic acid in water, according to the following linear gradient: 0 to 0.1 min: 90% B; 0.1 to 4.1 min: from 90% B to 74% B; 4.1 to 4.2 min: from 74% B to 5% B; 4.2 to 4.6 min: 5% B; 4.6 to 4.7 min: from 5% B to 90% B; 4.7-5.1 min; 90% B. Detection was performed using a Sciex AP16600 TOF mass spectrometer. Toxicokinetic parameters were estimated from concentration-time data using Phoenix pharmacokinetic software (version 6.4, Certara L.P.). A non-compartmental approach consistent with the bolus intravenous injection was used for parameter estimation.

No unscheduled deaths occurred during the study, and no DPI-4452 treatment-related clinical signs or local reactions were observed. The test item treatment led to unaffected body weights, weight gains, and food intake. Likewise, no treatment-related abnormalities were reported during ophthalmic examinations, urinalysis, blood biochemistry, coagulation, and hematology investigations. There were no related organ weight changes and macroscopic and microscopic observations at early and late necropsies. Jacketed external telemetry was used to evaluate the potential cardiovascular and respiratory effects in the Subset B. Cardiorespiratory telemetry recordings were performed during the pre-test, Day 1 and 14. The electrocardiogram (ECG) parameters (heart rate, PQ interval duration, QRS complex duration, and QT interval duration) were collected continuously after dosing over 10-minute periods up to 6 h and over 15-minute periods up to 24 h. Miyazaki's QT correction method (Miyazaki, H. & Tagawa, M. Japanese Association for Laboratory Animal Science 2002, 51, 465-75) was used to correct the influence of heart rate. ECG abnormalities were checked over one minute at pre-dose, around Tmax (5, 15, and 30 min), and 24 h after the test item administration. Arterial blood pressure parameters (systolic and diastolic blood pressure and mean arterial pressure) were measured continuously during consecutive 30-minute periods at pre-dose and for up to 6 h after dosing and over consecutive 60-minute periods up to 24 h after dosing. The respiratory rate was monitored on the pre-test, Day 1 and Day 14 at 0.5, 1, 2, 3, 4, 6, 8, 12, 16, 20, and 24 h. There was no related effect on cardiovascular or respiratory parameters, including heart rate, arterial blood pressure, ECG parameters, qualitative abnormalities, or the respiratory rate at any dose or timepoints. The potential effects on central nervous system parameters were evaluated under restrained and non-restrained conditions using a FOB in Subset A. Neurologic, autonomic, and behavioral investigations were performed in the pre-test, pre-dose, and 1 hour after dosing on Day 1. No effects were observed on the central nervous system, including neurologic, autonomic, and behavioral domains.

The TK parameters of DPI-4452 are summarized in Table 36. No appreciable difference in DPI-4452 exposure was observed between male and female dogs at all dose levels. An apparent, more than dose-proportional increase in area under the plasma concentration-time curve (AUC) was observed between the low and the intermediate dose levels (at least partly explained by the fewer quantified points at the low dose), whereas a dose-proportional increase in AUC was observed between the intermediate and high dose levels (FIG. 31).

In conclusion, a single i.v. bolus injection of DPI-4452 up to 400 μg/kg was well tolerated in dogs. There were no treatment-related findings reported in clinical pathology, histopathology, FOB evaluation, and cardiovascular and respiratory safety pharmacology assessment. Based on these results, the NOAEL was considered 400 μg/kg. At the NOAEL, the mean AUCtlast values were 854 and 771 ng·h/mL for males and females, respectively. The dog's NOAEL of 400 μg/kg has a human equivalent dose of 13.3 mg, considering a 60 kg human. Since the total ligand mass dose will be 500 μg/patient, the NOAEL covers more than 25 times the predicted human dose.

TABLE 36
Summary (mean ± SD; n = 3) of toxicokinetic parameters
of DPI-4452 following a single i.v. bolus injection of DPI-
4452 at 16, 80 or 400 μg/kg in male and female beagle dogs

| | | | | | | | |
|---|---|---|---|---|---|---|---|
| | | | | AUCₗₐₛₜ/ | | | |
| | | | | Dose | | | |
| | C_{5 min} | tₗₐₛₜ* | AUCₗₐₛₜ | (h · ng/mL)/ | t_{1/2}* | CL | Vss |
| Sex | (ng/mL) | (h) | (h · ng/mL) | (μg/kg) | (h) | (mL/min/kg) | (L/kg) |

Dose level: 16 μg/kg

| | | | | | | | |
|---|---|---|---|---|---|---|---|
| Female | 37.7 | 1 [1-1] | 12.4 | 0.774 | 0.239 | 21.2 | 0.30 |
| | (6.83) | | (2.13) | (0.133) | | (4.0) | (0.06) |
| Male | 43.5 | 1 [1-1] | 14.0 | 0.873 | 0.259 | 18.3 | 0.26 |
| | (2.75) | | (1.29) | (0.0806) | | (1.9) | (0.04) |
| F/M | n.a. | n.a. | 1.13 | n.a. | n.a. | n.a. | n.a. |

Dose level: 80 μg/kg

| | | | | | | | |
|---|---|---|---|---|---|---|---|
| Female | 346 | 3 [2-3] | 144 | 1.80 | 0.386 | 9.27 | 0.21 |
| | (34.8) | | (15.3) | (0.192) | | (1.0) | (0.02) |
| Male | 332 | 2 [2-3] | 132 | 1.65 | 0.361 | 10.1 | 0.21 |
| | (14.7) | | (5.49) | (0.0687) | | (0.4) | (0.01) |
| F/M | n.a. | n.a. | 0.917 | n.a. | n.a. | n.a. | n.a. |

Dose level: 400 μg/kg

| | | | | | | | |
|---|---|---|---|---|---|---|---|
| Female | 1760 | 3 [3-3] | 771 | 1.93 | 0.363 | 8.63 | 0.19 |
| | (180) | | (40.6) | (0.101) | | (0.4) | (0.02) |
| Male | 1840 | 3 [3-3] | 854 | 2.13 | 0.395 | 7.88 | 0.19 |
| | (257) | | (120) | (0.300) | | (1.0) | (0.02) |
| F/M | n.a. | n.a. | 1.11 | NA | n.a. | n.a. | n.a. |

AUCₗₐₛₜ = area under the concentration curve to last measurable concentration, n.a. = not applicable, SD = Standard Deviation, C_{5 min} = measured concentration at 5 min post injection; F/M = Female-to-Male AUC ratio, tₗₐₛₜ = time to last measureable concentration.
*Median [Min-Max] for tₗₐₛₜ, harmonic mean for t_{1/2}.

### Example 10: Pharmacokinetic Characterization in Healthy Mice

The pharmacokinetics of DPI-4452 after a single intravenous (i.v.) dose were investigated in healthy mice. DPI-4452 was administered to male CD-1 mice via the i.v. route at 0.7 and 5.5 mg/kg. Plasma and urine samples collected after dosing were analyzed using a LC-HRMS (liquid chromatography coupled with high-resolution mass spectrometry) assay method. Concentration values were used for pharmacokinetic calculations.

DPI-4452 was formulated in 0.1 M HEPES pH 7.1 at the final concentrations of 0.35 and 2.75 mg/mL for dosing at 0.7 and 5.5 mg/kg, respectively. Male CD-1 mice (3 animals per time point and per dose level; overall, 28.2-37.6 g body weight) were administered intravenously in the tail vein in a 2 mL/kg dosing volume. Blood samples were collected in K₂EDTA tubes at 5 min, 15 min, 30 min, 1 h, 2 h, 4 h, 8 h, and urine was collected over 8 hours post dose. The blood samples were kept on wet ice until plasma separation by centrifugation at room temperature; 10 min; 2500 G, which was performed within 60 min after sampling. The plasma samples were transferred into plastic tubes, frozen and stored at −80° C. until analysis.

Plasma and urine samples were analyzed by LC-HRMS after solid phase extraction, using a stable isotope-labeled internal standard (¹³C₆, ¹⁵N-DPI-4452). Chromatographic separation was run on a Waters Acquity HSS T3 C18 2.1×50 mm, 1.8 μm column. Detection was achieved on a Sciex AP16600 TOF mass spectrometer. The lower limit of quantification was 1.00 ng/mL in plasma and urine.

The non-compartmental (NCA) pharmacokinetic analysis of plasma concentration data was conducted using Phoenix 64 WinNonlin (Build 8.3.3.33) software. Nominal doses were used for all animals. The terminal phase half-life (T_{1/2}) was calculated by least-squares regression analysis of the terminal linear part of the log concentration-time curve. The area under the plasma concentration-time curve (AUC) was determined with the linear trapezoidal rule for increasing values and log trapezoidal rule for decreasing values up to the last measurable concentration.

### Results

After i.v. administration of DPI-4452 at a dose of 0.7 mg/kg, plasma concentrations declined in an apparent bi-phasic manner with a terminal elimination half-life of 0.280 h. Systemic exposure as measured by AUC_{0-inf} was 424 h*ng/mL. Clearance (CL) and volume of distribution (V_{d}) were 27.5 mL/min/kg and 0.408 L/kg, respectively. On average, 3.57% of the injected DPI-4452 dose was retrieved from urine unchanged over 8 hours.

After i.v. administration of DPI-4452 at a dose of 5.5 mg/kg, plasma concentrations declined in an apparent bi-phasic manner with a terminal elimination half-life of 0.465 h. Systemic exposure as measured by AUC_{0-inf} was 2820 h*ng/ml. Clearance (CL) and volume of distribution (V_{d}) were 32.5 ml/min/kg and 0.455 L/kg, respectively. On average, 8.89% of the injected DPI-4452 dose was retrieved from urine unchanged over 8 hours.

### Example 11: Pharmacokinetic Characterization in Healthy Dogs

The pharmacokinetics of DPI-4452 after a single intravenous (i.v.) dose was investigated in healthy dogs. DPI-4452 was administered to male beagle dogs via the i.v. route at 0.1 and 0.8 mg/kg. Plasma and urine samples collected after dosing were analyzed using a LC-HRMS (liquid chromatography coupled with high-resolution mass spectrometry) assay method. Concentration values were used for pharmacokinetic calculations.

DPI 4452 was formulated in 0.1 M HEPES pH 7.0 at the final concentrations of 0.05 and 0.4 mg/mL for dosing at 0.1 and 0.8 mg/kg, respectively. Male beagle dogs (3 animals per dose level; overall 8.9 to 12.4 kg body weight) were administered intravenously (bolus) in a 2 mL/kg dosing volume. Blood samples were collected in K₂EDTA tubes at 5 min, 15 min, 30 min, 1 h, 2 h, 4 h, 6 h, 24 h, and urine was collected over 24 hours post dose. The blood samples were kept on wet ice until plasma separation by centrifugation within 60 min after sampling. The plasma samples were transferred into plastic tubes, frozen and stored at −80° C. until analysis.

Plasma and urine samples were analyzed by LC-HRMS after solid phase extraction, using DPI-4501 as internal standard. Chromatographic separation was run on a Waters Acquity HSS T3 C18 2.1×50 mm, 1.8 μm column. Detection was achieved on a Sciex AP16600 TOF mass spectrometer. The lower limit of quantification was 2.00 ng/mL in urine and plasma.

The NCA pharmacokinetic analysis of plasma concentration data was conducted using Phoenix WinNonlin 6.3 software. Nominal doses were used for all animals. The terminal phase half-life (T1/2) was calculated by least-squares regression analysis of the terminal linear part of the log concentration-time curve. The area under the plasma concentration-time curve (AUC) was determined with the linear trapezoidal rule for increasing values and log trapezoidal rule for decreasing values up to the last measurable concentration.

### Results

DPI-4452 plasma concentration was quantifiable up to 2 h and 4 h after i.v. administration at 0.1 and 0.8 mg/kg, respectively.

Systemic exposure as measured by Cₘₐₓ and AUCinf after intravenous administration at 0.1 mg/kg was 377 ng/mL and 133 hr*ng/mL.

Systemic exposure as measured by Cₘₐₓ and AUCinf after intravenous administration at 0.8 mg/kg was 4430 ng/mL and 1760 hr*ng/mL.

Following i.v. administration at 0.1 mg/kg, DPI-4452 showed low inter-animal variability. Clearance was 12.7 mL/min/kg and volume of distribution was 0.26 L/kg with a terminal half-life (T1/2) of 0.38 h.

Following i.v. administration at 0.8 mg/kg, DPI-4452 showed low inter-animal variability. Clearance was 7.6 mL/min/kg and volume of distribution was 0.19 L/kg with a terminal half-life (T1/2) of 0.48 h.

The mean amount of unchanged DPI-4452 excreted in urine after intravenous administration at a dose level of 0.1 mg/kg was 26.8 ng/mL, corresponding to 0.29% of dose excreted unchanged. This results in an estimated renal clearance of 34.4 μL/min/kg.

The mean amount of unchanged DPI-4452 excreted in urine after intravenous administration at a dose level of 0.8 mg/kg was 191 ng/mL, corresponding to 0.14% of dose excreted unchanged. This results in an estimated renal clearance of 11.4 μL/min/kg.

### Example 12: Allometry and Prediction of Human Exposure and PK Parameters

Based on available mouse and dog PK data, human PK parameters (CL, V_{d} and T_{1/2}) for DPI-4452 were predicted by allometry (Zou P et al., AAPS Journal, 14:262-281 (2012)). From the variety of CL, V_{d} and T_{1/2} determined in the animal studies, the highest and lowest values for each species were retained, and allometry was conducted on all the combinations to obtain a range for each predicted human PK parameter. The following predicted human PK parameters were obtained: clearance (CL) 3.57-13.0 mL/min/kg; volume of distribution (V_{d}) 189-455 mL/kg; terminal half-life (T1/2) 0.17-1.47 h.

Simulations of human PK profiles were done with the open-source software PK Tool in order to estimate a value for C5 min, the plasma concentration of DPI-4452 in humans at 5 min post dose (practical Cₘₐₓ value after i.v. dosing), after a single intravenous dose of 250 μg. The missing input parameters were dealt with as follows. Regarding blood:plasma concentration ratio, the frequent default values of 1.0 and 0.8 were considered and found to make no difference to the outputs, so it was left at 1.0. Plasma protein binding: a free fraction fᵤₚ of 1 was considered in order to estimate the highest C5 min values.

The following predicted maximum human concentration was obtained: C5 min of 7.91-19.0 ng/mL after an intravenous dose of 250 μg; 15.8-38.0 ng/mL after an intravenous dose of 500 μg (assuming dose-linearity between 250 and 500 μg).

### Example 13:Prevalence of CAIX Expression in CRC, PDAC, Sq. NSCLC, SCCHN, TNBC and ccRCC Cancers

CAIX protein expression was assessed using a validated immunohistochemistry assay (IHC) with an anti-CAIX antibody (M75) on a panel of 30 ccRCC, 70 PDAC, 80 Sq. NSCLC, 60 SCCHN, 95 TNBC and 85 CRC tumor specimens as well as healthy tissue. H-score was calculated for each individual sample.

The IHC method was adapted from Rasheed S. et al. (Pathol Res Pract. 2009, 205(1), 1-9). Tissue Microarrays (TMAs) containing colon carcinoma specimens (#BC000110), healthy normal colon tissue (#C0727), normal lung tissue (#LCN241), lung SCC (#LC808b), mixed pancreatic tissues (#PA482, #PA805c), a breast cancer (#BR1901), a head and neck cancer (#HN601d), a normal multi-organ (#FDA999w), ccRCC specimens and non-tumoral adjacent kidney tissue (#KD601a) panel were purchased from US Biomax and used for validation.

The CA9 (mouse clone M75) assay was evaluated on a semi-quantitative scale, and the percentage of tumor cells or normal cells staining at each of the following four levels was recorded: 0 (no staining), 1+ (weak staining), 2+ (moderate staining) and 3+(strong staining). A tumor or normal sample was considered positive if at least 1% of cells demonstrated positive expression. The subcellular localization (SCL) of staining was noted for positive samples.

### Pathologist Tumor H-Score

The Pathologist H-Score was calculated based on the summation of the product of percent of cells stained at each staining intensity using the following equation: (3×% cells staining at 3+)+ (2×% cells staining at 2+)+ (1×% cells staining at 1+).

The measured CAIX prevalence is shown in the table below with respect to each tumor type.

TABLE 37
Prevalence of CAIX expression in CRC, Sq.
NSCLC, PDAC, SCCHN, TNBC and ccRCC cancers

| | H-Score | | | Sq. | | | |
|---|---|---|---|---|---|---|---|
| Tissue | (max = 300) | CRC | ccRCC | NSCLC | PDAC | SCCHN | TNBC |
| Malignant | >150 | 29% | 83% | 19% | 40% | 23% | 19% |
| | | (25/85) | (25/30) | (15/79) | (26/65) | (14/60) | (10/54) |
| | >100 | 35% | 83% | 20% | 51% | 33% | 24% |
| | | (30/85) | (25/30) | (16/79) | (33/65) | (20/60) | (10/54) |
| | >40 | 56% | 87% | 42% | 60% | 62% | 37% |
| | | (48/85) | (26/30) | (33/79) | (39/65) | (37/60) | (10/54) |
| Healthy | >40 | 0% | 0% | 0% | 0% | n.d. | n.d. |
| control | | (0/21) | (0/30) | (0/22) | (0/30) | | |

### Example 14: Multicenter, Open-Label, Non-Randomized Phase 1/2 Study to Assess Safety, Tolerability, and Imaging Characteristics of [⁶⁸Ga]Ga-DPI-4452 and to Assess Safety, Tolerability, and Efficacy of [¹⁷⁷Lu]Lu-DPI-4452 in Patients with Unresectable Locally Advanced or Metastatic Solid Tumors

Overview of study design: This is an open-label, non-randomized, multicenter Phase 1/2 study of [⁶⁸Ga]Ga-DPI-4452 and [¹⁷⁷Lu]Lu-DPI-4452 in patients with unresectable, locally advanced, or metastatic solid tumors in the following 3 tumor types:
Clear cell renal cell cancer (ccRCC): patients must have received at least one line containing antiangiogenic tyrosine kinase inhibitors (TKI) treatment and at least one line containing Immune Checkpoint Inhibitor treatment in the metastatic setting, meaning at least two lines of treatment in metastatic setting; or
Pancreatic ductal adenocarcinoma (PDAC): patients must have received at least one line of platinum- and/or gemcitabine-based regimen; or
Colorectal cancer (CRC): patients must have received at least one line of FOLFIRINOX or FOLFOX/FOLFIRI in two lines in combination with anti-Vascular Endothelial Growth Factor (VEGF) or anti-Epidermal Growth Factor Receptor (EGFR).

The study is conducted in three parts:

### Phase 1:

Study Part A: an imaging part enrolling patients receiving a single dose 185 MBq (5 mCi) (±20%) of [⁶⁸Ga]Ga-DPI-4452 to evaluate the safety, tolerability, pharmacokinetics (PK) dosimetry, and imaging characteristics of [⁶⁸Ga]Ga-DPI-4452 by positron emission tomography (PET)/computed tomography (CT) scan assessed by central reading. All patients will be followed up for 7 days post-[⁶⁸Ga]Ga-DPI-4452 dose.
As the effect of antiangiogenic TKI on [⁶⁸Ga]Ga-DPI-4452 imaging by PET is unknown, patients who are on treatment with antiangiogenic TKI or received one within 7 days before the [⁶⁸Ga]Ga-DPI-4452 administration and show insufficient 68Ga uptake by PET, may be allowed to repeat the procedure after a wash-out period of 7 days for the antiangiogenic TKI following agreement with the Sponsor.
This part will include up to 15 patients including the tumor types ccRCC, PDAC, or CRC and approximately 3-5 patients per tumor type.
The totality of data including safety, dosimetry, imaging and available pharmacokinetic (PK) data for each tumor type will be assessed by the Safety Monitoring Committee (SMC) before Study Part B will start for the respective tumor type. Part B for ccRCC may start before the completion of the patients with other tumor types in Part A.
Patients in Part A with PET-positive tumors as defined in the inclusion criterion n. 14 for Part B and Part C may be allowed to participate in Part B or Part C, once these parts open for enrollment and if they meet eligibility criteria for Part B or Part C, respectively. For these patients, all screening assessments for Part B and Part C will be performed, including the [⁶⁸Ga]Ga-DPI-4452 injection and PET/CT scan if >2-month-old at the time of rescreen.
Study Part B: is a [¹⁷⁷Lu]Lu-DPI-4452 therapeutic dose escalation part that will start in ccRCC patients after confirmation of safety, favorable biodistribution, and dosimetry of [⁶⁸Ga]Ga-DPI-4452 in patients in Part A.
During the screening period, patients will undergo PET/CT imaging after receiving a single dose of [⁶⁸Ga]Ga-DPI-4452. Patients with PET-positive tumors (according to the inclusion criterion n. 14), as assessed by central reading, are eligible to start treatment with [¹⁷⁷Lu]Lu-DPI-4452 administered on Day 1 of each 28-day cycle for up to a maximum of 8 cycles.The SMC will define the maximum number of cycles that may be administered per cohort, which will depend on the estimated maximum cumulative dose of [¹⁷⁷Lu]Lu-DPI-4452 and it will be documented in the SMC minutes.
The aim of this study part is to define the Recommended Phase 2 Dose (RP2D) (Maximum Tolerated Dose [MTD] or lower dose), to assess safety, tolerability, PK, dosimetry, and preliminary antitumor activity of [¹⁷⁷Lu]Lu-DPI-4452 for each of the 3 tumor types.
The dose escalation will initially be conducted in the group of ccRCC patients. The starting dose will be 100 mCi and the dose is anticipated to be escalated by 100 mCi increments for subsequent cohorts, up to a maximum dose of 500 mCi per cycle. Intra-patient dose escalation will not be allowed, except for the first cohort of the ccRCC tumor type, where patients will receive [¹⁷⁷Lu]Lu-DPI-4452 at a starting dose of 100 mCi on Day 1 of the first cycle and, once [¹⁷⁷Lu]Lu-DPI-4452 dose of 100 mCi and the increase by 100 mCi are considered safe by the SMC. The individual patients in ccRCC cohort 1 must not have experienced a DLT and must have fully recovered from all treatment-related AEs or returned to baseline values before continuing with 200 mCi on Day 1 of all following cycles.
Once the RP2D for the ccRCC group has been defined, dose escalation in the groups of CRC and PDAC patients will start. The initial dose for these tumor types will be at least one dose level below the RP2D defined for the ccRCC group.
For all groups, each dose cohort will enroll between 2 and 6 evaluable patients. It is estimated that a total of approximately 42 evaluable patients will be enrolled in Part B of the study. Patients will be enrolled in a sequential manner with at least 7 days between dosing of first and the second patient at each new dose level.
At the end of each dose cohort, the totality of data including safety, efficacy, dosimetry, and available PK data will be assessed by the SMC. The dose escalation for each patient group will be supported by an adaptive Bayesian modified continual reassessment method—escalation with overdose control (mCRM-EWOC).

### Phase 2:

Study Part C: this part will start separately for each of the 3 tumor types once the RP2D has been defined for the respective tumor type in Part B. All patients will be screened including PET/CT scan after receiving a single dose of [⁶⁸Ga]Ga-DPI-4452. Eligible patients with PET-positive tumors according to the inclusion criterion n. 14 as assessed by central reading, will receive repeated doses of [¹⁷⁷Lu]Lu-DPI-4452 administered on Day 1 of each 28-day cycle. The maximum number of cycles will be defined by the SMC in Part B depending on the dose per cycle identified as RP2D and the resulting maximum cumulative dose, and it will be documented in the SMC meeting minutes.
The aim of this study part is to assess efficacy, safety, and tolerability of [¹⁷⁷Lu]Lu-DPI-4452 and confirm the RP2D dose for the 3 tumor types.
This part will include up to 30 patients in each of the 3 tumor types (up to 90 patients overall in Part C).

For the purpose of this study, a treatment cycle is defined as 28 days. [¹⁷⁷Lu]Lu-DPI-4452 dose is administered on Day 1 of each treatment cycle.

### Study Population:

Patients with unresectable locally advanced or metastatic solid tumors:
ccRCC: patients must have received at least one line containing TKI treatment and at least one line containing Immune Checkpoint Inhibitor treatment in metastatic setting, meaning at least two lines of treatment in metastatic setting; or
PDAC: patients must have received at least one line of platinum- and/or gemcitabine-based regimen; or
CRC: patients must have received at least one line of FOLFIRINOX or FOLFOX/FOLFIRI in two lines in combination with anti-VEGF or anti-EGFR.

### Inclusion Criteria:

Study Part A:
1) Histologically or cytologically confirmed, unresectable locally advanced or metastatic solid tumors of:
   ccRCC: patients must have received at least one line containing TKI treatment and at least one line containing Immune Checkpoint Inhibitor treatment in metastatic setting, meaning at least two lines of treatment in metastatic setting; or
   PDAC: patients must have received at least one line of platinum- and/or gemcitabine-based regimen; or
   CRC: patients must have received at least one line of FOLFIRINOX or FOLFOX/FOLFIRI in two lines in combination with anti-VEGF or anti-EGFR.
2) Age ≥18 years
3) Written informed consent, dated and signed by the patient prior to any study-specific procedure
4) For patients with CRC or PDAC: availability of fresh biopsy, OR an archival biopsy/surgical specimen of the tumor (preferably, taken after last prior line of therapy)
5) Presence of at least 1 non-irradiated tumor lesion detected at conventional imaging (CT/MRI) documented within 4 weeks prior to the [68Ga]Ga-DPI-4452 administration
6) Measurable disease as per RECIST v1.1
7) Patient Eastern Cooperative Oncology Group (ECOG) performance status 0-2
8) Adequate bone marrow reserve and organ function as demonstrated by complete blood count, and biochemistry in blood and urine at Screening
   Adequate blood counts:

$$Hemoglobin \geq 8 g / dL$$

$$Absolute neutrophil count ( ANC ) \geq 1. \times 109 / mL$$

$$Platelets \geq 50 \times 109 / mL$$

Adequate hepatic function:
   Aspartate or alanine aminotransferase or alkaline phosphatase (AST, ALT, ALP) ≤2.5×upper limit of normal (ULN) (≤5×ULN if patient has liver metastases)
Bilirubin ≤1.5×ULN (up to 2.0×ULN is allowed if the direct bilirubin level is normal, and the elevation is limited to indirect bilirubin)
   Adequate renal function:
   For PDAC and CRC patients: estimated glomerular filtration rate (eGFR) >50 mL/min/1.73 m2 (as determined by the Chronic Kidney Disease-Epidemiology Collaboration [CKD-EPI]formula)
   For ccRCC patients: eGFR >40 mL/min/1.73 m2 (CKD-EPI)
9) Women of childbearing potential (defined as all women physiologically capable of becoming pregnant) must have a negative serum pregnancy test at screening and must not be breastfeeding. Additionally, they must agree to use highly effective methods of contraception from informed consent form (ICF) signature for 180 days after the [⁶⁸Ga]Ga-DPI-4452 injection.
Sexual abstinence is acceptable only if it is consistent with the preferred and usual lifestyle of the patient. Periodic abstinence (e.g., calendar, ovulation, symptothermal, or post-ovulation methods) and withdrawal are not acceptable methods of birth control.
   Postmenopausal. A postmenopausal state is defined as no menses for 12 months without an alternative medical cause. A high follicle-stimulating hormone (FSH) level at screening (>40 IU/L) in the postmenopausal range may be used to confirm a postmenopausal state in women not using hormonal contraception or hormonal replacement therapy; however, in the absence of 12 months of amenorrhea, patients will be considered of childbearing potential.
   Permanently sterile. Permanent sterilization methods include hysterectomy, bilateral salpingectomy, and bilateral oophorectomy at least 6 months prior to first dosing or documented congenital sterility.
   Highly effective methods of contraception include combined (estrogen- and progestogen-containing) hormonal contraception associated with inhibition of ovulation (oral, intravaginal, transdermal), progestogen-only hormonal contraception associated with inhibition of ovulation (oral, injectable, implantable), intrauterine device, intrauterine hormone-releasing system, bilateral tubal occlusion/ligation procedures, vasectomized partner, and sexual abstinence during the entire study duration if in adequation with the patient usual lifestyle. Periodic abstinence (e.g., calendar, symptothermal, post-ovulation methods) is not considered as a highly effective method and is not acceptable.
10) Sexually active men must agree to use a condom during intercourse, refrain from fathering a child, and donating sperm during treatment period and for 90 days after stopping treatment. Female partners of childbearing potential must agree to practice sexual abstinence or be under highly effective birth control method from ICF signature to 90 days after the [⁶⁸Ga]Ga-DPI-4452 injection.
Study Part B and Part C:
1) Histologically or cytologically confirmed progressive, unresectable locally advanced or metastatic solid tumors of:
   ccRCC: patients must have received at least one line containing TKI treatment and at least one line containing Immune Checkpoint Inhibitor treatment in metastatic setting, meaning at least two lines of treatment in metastatic setting; or
   PDAC: patients must have received at least one line of platinum- and/or gemcitabine-based regimen; or
   CRC: patients must have received at least one line of FOLFIRINOX or FOLFOX/FOLFIRI in two lines in combination with anti-VEGF or anti-EGFR.
2) Age ≥18 years
3) Written informed consent, dated and signed by the patient prior to any study-specific procedure
4) For patients with CRC and PDAC availability of fresh biopsy, OR an archival biopsy/surgical specimen of the tumor (preferably, taken after last prior line of therapy)
5) Presence of at least 1 non-irradiated tumor lesion detected at conventional imaging (CT/MRI) documented after or during the last anticancer therapy and within 4 weeks preceding the [⁶⁸Ga]Ga-DPI-4452 administration
6) Measurable disease per RECIST v1.1
7) Patient ECOG performance status 0-1
8) Life expectancy >6 months
9) Adequate bone marrow reserve and organ function as demonstrated by complete blood count, and biochemistry in blood and urine at Screening
   Adequate blood counts:

$$Hemoglobin \geq 9 g / dL$$

$$ANC \geq 1.5 \times 109 / mL$$

$$Platelets \geq 100 \times 109 / mL$$

Adequate hepatic function:
   AST, ALT or ALP ≤2.5×ULN (≤5×ULN if patient has liver metastases)
   Bilirubin 51.5×ULN (up to 2.0×ULN is allowed if the direct bilirubin level is normal, and the elevation is limited to indirect bilirubin)
Adequate renal function:
   For PDAC and CRC patients: eGFR >50 mL/min/1.73 m2 (CKD-EPI)
   For ccRCC patients: eGFR >40 mL/min/1.73 m2 (CKD-EPI)
Adequate coagulation:
   International normalization ratio or prothrombin time ≤1.5×ULN and no history of major thrombotic or clinically relevant major bleeding event in the past 6 months putting the subject at high risk of bleeding during the study as assessed by the Investigator
10) Women of childbearing potential (defined as all women physiologically capable of becoming pregnant) must have a negative serum pregnancy test at screening and must not be breastfeeding. Additionally, they must agree to use highly effective methods of contraception from ICF signature for 180 days after the [⁶⁸Ga]Ga-DPI-4452 injection and for 180 days after [¹⁷⁷Lu]Lu-DPI-4452 infusion.
   Sexual abstinence is acceptable only if it is consistent with the preferred and usual lifestyle of the patient. Periodic abstinence (e.g., calendar, ovulation, symptothermal, or post-ovulation methods) and withdrawal are not acceptable methods of birth control.
      Postmenopausal. A postmenopausal state is defined as no menses for 12 months without an alternative medical cause. A high FSH level at screening (>40 IU/L) in the postmenopausal range may be used to confirm a postmenopausal state in women not using hormonal contraception or hormonal replacement therapy; however, in the absence of 12 months of amenorrhea, patients will be considered of childbearing potential.
      Permanently sterile. Permanent sterilization methods include hysterectomy, bilateral salpingectomy, and bilateral oophorectomy at least 6 months prior to first dosing or documented congenital sterility.
      Highly effective methods of contraception include combined (estrogen- and progestogen-containing) hormonal contraception associated with inhibition of ovulation (oral, intravaginal, transdermal), progestogen-only hormonal contraception associated with inhibition of ovulation (oral, injectable, implantable), intrauterine device, intrauterine hormone-releasing system, bilateral tubal occlusion/ligation procedures, vasectomized partner, and sexual abstinence during the entire study duration if in adequation with the patient usual lifestyle. Periodic abstinence (e.g., calendar, symptothermal, post-ovulation methods) is not considered as a highly effective method and is not acceptable.
11) Sexually active men must agree to use a condom during intercourse, refrain from fathering a child and donating sperm during treatment period and for 90 days after stopping treatment. Female partners of childbearing potential must agree to practice sexual abstinence or be under highly effective birth control method from ICF signature to 90 days after the last [¹⁷⁷Lu]Lu-DPI-4452 infusion.
12) Radiographically documented disease progression or recurrence during or after the last systemic treatment regimen
13) Patients for whom no standard therapy is available, or in the opinion of the Investigator, are unlikely to tolerate standard care.
14) ≥75% of the lesions detected by standard imaging (CT scan, MRI) assessed by central reading must be positive by [⁶⁸Ga]Ga-DPI-4452 PET.
15) Patients with known central nervous system (CNS) metastasis will be eligible if they are clinically stable, and asymptomatic. Patients must have completed primary CNS therapy more than 4 weeks before treatment start (such as whole brain radiotherapy, stereotactic radiosurgery, or complete surgical resection). Low doses of steroids for the purposes of maintaining neurological integrity (not exceeding 10 mg/day) are allowed. For patients with CNS metastasis (or a history of CNS metastasis), baseline and subsequent radiological imaging must include evaluation of the brain (MRI preferred or CT with contrast).

### Investigational Product(s), Dose and Mode of Administration:

[⁶⁸Ga]Ga-DPI-4452 solution for injection:
DPI-4452 kits for radiopharmaceutical preparation will be supplied to the radiopharmacy at study sites (or central radiopharmacy facilities, where applicable) as a cold kit to be used for the on-site manufacturing of the [⁶⁸Ga]Ga-DPI-4452 solution for injection to be administered to the patients.
The peptide-ligand precursor must be radiolabeled with ⁶⁸Ga at the radiopharmacy of the site or at a central radiopharmacy facility to generate the [⁶⁸Ga]Ga-DPI-4452 solution for injection, according to the applicable local regulations and as described in the Pharmacy Manual.
The radiolabeled diagnostic product will be manufactured, quality controlled, released, and administered within the defined shelf-life to secure an administered activity dose within the optimal range 185 MBq (±20%) (5 mCi±1 mCi). Administration of [⁶⁸Ga]Ga-DPI-4452 must be performed in accordance with national and/or local radiation and safety requirements by trained personnel.
The dose of [⁶⁸Ga]Ga-DPI-4452-solution for injection is to be injected as a slow intravenous injection. The time of the start of the administration must be recorded, and total activity administered must be measured (GBq) and documented in the patient's source documents.
[¹⁷⁷Lu]Lu-DPI-4452 solution for infusion:
[¹⁷⁷Lu]Lu-DPI-4452 solution for infusion will be provided as a pre-formulated ready-to-use solution by a centralized manufacturing facility. Treatment with [¹⁷⁷Lu]Lu-DPI-4452 must be performed in accordance with national and/or local radiation and safety requirements by trained personnel. Patient should be well-hydrated. A saline flush must be administered to ensure patency of the intravenous line before starting [¹⁷⁷Lu]Lu-DPI-4452 infusion, and it must also be administered at the end of the infusion. The [¹⁷⁷Lu]Lu-DPI-4452 infusion should be completed within 30 min. The time of the start and end of the administration must be recorded, and total activity administered must be measured (GBq) and documented in patient's source documents.
A decision to order [¹⁷⁷Lu]Lu-DPI-4452 should be communicated to the Sponsor or designee prior to the planned administration for each cycle, considering 10-16 business days for the delivery.
All patients in Part A, Part B and Part C will receive 1 dose of [⁶⁸Ga]Ga-DPI-4452 at 185 MBq (±20%) (5 mCi ±1 mCi).
Study Part B: In the first cohort of the ccRCC tumor type, patients will receive [177Lu]Lu-DPI-4452 at a starting dose of 100 mCi on Day 1 of the first cycle and, once [177Lu]Lu-DPI-4452 dose of 100 mCi and the increase by 100 mCi are considered safe by the SMC, will continue with 200 mCi on Day 1 of all following cycles. The next cohorts of this and other tumor types will receive [177Lu]Lu-DPI-4452 doses in 100 mCi increments, as agreed by the SMC.
Study Part C: [177Lu]Lu-DPI-4452 dosing will be based on the RP2D(s) defined in Part B.
For patients in Part B and Part C, dose reductions will be allowed for safety/tolerability reasons at any cycle, by 100 mCi for a maximum of 2 dose reductions and/or a minimum dose of 100 mCi. Dose delays due to toxicities up to 2 weeks will be allowed. If the treatment needs to be delayed for >2 weeks, the dose should be omitted and administered in the next cycle.

### Imaging, PK and Dosimetry:

Study Part A:
PET dosimetry assessments will be conducted based on time-activity curves (TAC), time integrated activity coefficients, absorbed doses, and effective dose. SUVmax will be used to determine the tumor lesions, defined as focal areas of abnormal uptake showing a higher SUVmax than background region. Identification of the timepoints post-injection with the highest observed number of lesions and the highest SUVmax, SUVmean values and the tumor to background ratios for each SUV will be determined.
Whole-body scans will be performed at 15 min, and at 1, 2, 3-4 h post-[⁶⁸Ga]Ga-DPI-4452 injection. Region of Interest will be drawn on the whole-body PET/CT images over the critical organs and tumor lesions to generate TACs, calculate the TBR, and assess the radioactivity residence times.
PK blood samples for radioactivity will be collected pre-injection, approximately 5, 10, 40 min; 1, 2, 4, and 6 h post-injection. Plasma samples for DPI-4452 (and potential metabolites) PK evaluation will be collected at the same timepoints. Urine samples will be collected pre-injection, and up to 6 h post-injection for radioactivity and DPI-4452 PK evaluation.
Study Part B & Part C:
All patients will undergo whole-body [⁶⁸Ga]Ga-DPI-4452 PET/CT imaging optimized for time (up to 2 PET timepoints) according to the data analysis from Part A of the study. Optimal schedule of the imaging has not been established; some adaptation of these timelines might be required during the Part B and Part C of the study and will be decided by the dosimetry expert and the Sponsor and will be documented in the Imaging Manual.
Qualitative visual analysis of the total number of lesions identified by PET, and semi-quantitative analysis by means of SUVmax and SUVmean values per dose and timepoints will be conducted.
Eligible patients with PET-positive tumors according to the inclusion criterion n. 14 as assessed by central reading, will receive treatment with [¹⁷⁷Lu]Lu-DPI-4452. Tumor assessments as per RECIST v1.1 will be performed by the Investigator every 6 weeks for the first 3 assessments (up to 24 weeks) and then every 12 weeks until disease progression or end of study (EOS), whichever occurs first.
Full ¹⁷⁷Lu dosimetry will be performed in Cycle 1 in all patients in Part B and a subgroup (~10 patients in each tumor type) of Part C.
Each patient will have up to 4 scan timepoints (whole-body planar images and/or 3D SPECT/CT) acquired at Cycle 1 over a period of 7 days from the [¹⁷⁷Lu]Lu-DPI-4452 administration for the purpose of dosimetry. The site physician may order additional areas to be scanned as needed, based on the previous [⁶⁸Ga]Ga-DPI-4452 PET/CT scan.
The optimal schedule of the post-treatment whole-body scans (planar scintigraphy) and SPECT/CT will be established by the dosimetry expert and the Sponsor and will be documented in the Imaging Manual, which will be updated as needed.
PK assessments will be performed on all patients in Part B and in a subset of patients (approximately 10 patients/tumor type) in Part C. At Cycle 1, PK blood samples for radioactivity will be collected pre-infusion, approximately 5 min before the end of infusion, 20 min, 1 h, 2 h, 4 h, 8 h, 24 h, 48 h, 72 h post-infusion.
Plasma samples for PK of radioactivity and DPI-4452 (and potential metabolites) will be collected at the same timepoints. Urine samples will be collected pre-infusion, and post-infusion up to 72 h for radioactivity and DPI-4452 (and potential metabolites) PK evaluation.
[¹⁷⁷Lu]Lu-DPI-4452 metabolic stability in collected plasma and urine samples will be evaluated where technically feasible during Part B only.
At Cycles 2 and 3, blood and plasma samples will be drawn at approximately 5 min before the end of injection, and 20 min and 6 h post-injection for PK of radioactivity and DPI-4452.

### Definition of DLT:

Part B:
For the purposes of dose escalation decisions in Part B, each cohort will consist of 2 to 6 evaluable patients who will be treated with [¹⁷⁷Lu]Lu-DPI-4452 at the specified dose levels.
The DLT evaluation period is defined as Cycle 1 (i.e., 28 days) following the first [¹⁷⁷Lu]Lu-DPI-4452 administration.
To be evaluable for DLT assessment, a patient should have received at least 1 full dose (~10%) of [¹⁷⁷Lu]Lu-DPI-4452 and completed the first 28 days on study or experienced a DLT before completion of the DLT period.
A DLT is defined as an AE or abnormal laboratory value that fulfills all of the following criteria:
   For which a relationship to [¹⁷⁷Lu]Lu-DPI-4452 cannot be ruled out, e.g., is not primary related to the underlying disease, disease progression, intercurrent illness, or concomitant medications.
   Occurs during Cycle 1 (28 days) following the first [¹⁷⁷Lu]Lu-DPI-4452 administration.
   Meets at least one of the criteria included below:

| | New treatment-emergent AEs or worsening of previous AE |
|---|---|
| | (as per NCI-CTCAE v5.0) |
| Hemato- | Grade 4 neutropenia ≥7 consecutive days |
| logical | Febrile neutropenia ≥7 consecutive days |
| toxicity | Grade 4 thrombocytopenia ≥7 consecutive days, despite |
| | best supportive care |
| | Grade 3 thrombocytopenia complicated by a clinically |
| | significant bleeding event |
| | Grade 4 anemia ≥7 consecutive days, despite best |
| | supportive care |
| Liver | Drug-induced liver injuries meeting the criteria of Hy's law |
| toxicities | (AST or ALT >3 × ULN and total bilirubin >2 × ULN |
| | without any initial findings of cholestasis [elevated ALP] |
| | and when the increase cannot be attributed to another |
| | cause; for participants with hepatic metastases, AST |
| | or ALT >8 × ULN or AST or ALT >5 × ULN for ≥14 days) |
| | Bilirubin Grade ≥3 |
| | AST or ALT Grade 4 |
| Gastro- | Grade ≥3 nausea, vomiting, diarrhea or other symptoms |
| intestinal | suggestive of gastritis or duodenitis >3 consecutive days |
| toxicity | despite adequate supportive care. |
| Pancreatic | Grade ≥3 amylase/lipase increase associated with |
| toxicity | symptoms or clinical manifestations or radiological |
| | confirmation of pancreatitis. |
| Renal | Grade ≥3 decrease in creatinine clearance or Grade ≥3 |
| toxicity | serum creatinine increase ≥7 consecutive days or |
| | requiring hospitalization. |
| Cardiac | QTc prolongation defined as QTcF >500 ms, or a |
| toxicity | prolongation from baseline of >60 ms, confirmed by a |
| | second ECG triplicate |
| | Persistent arrhythmia and other persistent cardiac |
| | disorders Grade ≥3 |
| Other non- | Any other Grade ≥3 lasting ≥7 days |
| hemato- | |
| logical | |
| toxicities | |

### Treatment Duration:

Part A:
Eligible patients will receive 1 single [⁶⁸Ga]Ga-DPI-4452 dose of 185 MBq (±20%) for imaging purposes and will be followed up for 7 days.
Part B and Part C:
During screening, patients will receive 1 single [⁶⁸Ga]Ga-DPI-4452 dose of 185 MBq (±20%) for imaging purposes.
Patients who were not eligible for study treatment with [¹⁷⁷Lu]Lu-DPI-4452 but received [⁶⁸Ga]Ga-DPI-4452 will be followed up for 7 days. Eligible patients will start study treatment with [¹⁷⁷Lu]Lu-DPI-4452 on Day 1 of Cycle 1 and continue study treatment with [¹⁷⁷Lu]Lu-DPI-4452 on Day 1 of subsequent cycles.
In Part B, patients may receive [¹⁷⁷Lu]Lu-DPI-4452 for up to a maximum of 8 cycles. Two additional cycles may be given after Cycle 4 and Cycle 6; for each patient the decision to continue treatment will be based on safety and efficacy and if the following conditions are met (as per Investigator's judgment):
   [¹⁷⁷Lu]Lu-DPI-4452 is well tolerated.
   No evidence of disease progression (as per RECIST v1.1).
In Part C, the maximum number of [¹⁷⁷Lu]Lu-DPI-4452 cycles to be administered will be defined by the SMC in Part B depending on the dose per cycle identified as RP2D and the resulting maximum cumulative dose, and it will be documented in the SMC meeting minutes.
Patients will discontinue study treatment in case of unacceptable toxicity, disease progression, withdrawal of consent, or Investigator's decision. An end of treatment (EOT) visit will occur within 4 weeks (28 days) after the last dose of study treatment.

### Duration of Study Participation:

Part A:
Patients will be on study for approximately 3 weeks:
   Up to 2 weeks of screening assessments;
   A 1-day [⁶⁸Ga]Ga-DPI-4452 administration and imaging/dosimetry period; and
   A 7-day safety follow-up period after the administration.
Part B and Part C:
Patients are expected to be on study for:
   Up to 6-week screening period (up to 2 weeks for determination eligibility before [⁶⁸Ga]Ga-DPI-4452 administration and imaging and up to 4 weeks for imaging, dosimetry and [¹⁷⁷Lu]Lu-DPI-4452 ordering and shipping);
   A treatment period up to a maximum of 8 cycles;
   A safety follow-up period lasting 60 days after the last dose of study treatment; and
   A disease progression and/or survival follow-up period which will last until all patients have completed a minimum of 12 months follow-up after their last dose, have discontinued prematurely, or if the study is terminated prematurely, whichever occurs first.
Safety follow-up for Part B and Part C:
Patients who have received at least 1 dose of [¹⁷⁷Lu]Lu-DPI-4452 will be followed up for safety for 60 days following the last dose of [¹⁷⁷Lu]Lu-DPI-4452. Patients who were not eligible for study treatment with [¹⁷⁷Lu]Lu-DPI-4452 but received [⁶⁸Ga]Ga-DPI-4452 will be followed up for 7 days for safety and tolerability.
Disease progression follow-up for Part B and Part C:
Patients who discontinue study treatment as per planned treatment duration, as per Investigator or patient decision, or due to AE, will be followed up for progression of their disease every 12 weeks (±15 days).
The disease progression follow-up for each patient will last until progression of disease, patient withdrawal, death, discontinuation as per Investigator's decision, start of a new subsequent antineoplastic therapy, or end of the study, whichever occurs first.
Survival follow-up for Part B and Part C:
All patients treated with RP2D will be followed for survival every 12 weeks (±15 days) until patient withdrawal, death, or end of the study, whichever occurs first.

### End of Study:

Part A:
The EOS at patient level will take place 7 days after administration of [⁶⁸Ga]Ga-DPI-4452.
Part B and Part C:
The EOS at patient level occurs upon death, loss to follow-up, withdrawal of consent, discontinuation as per Investigator's decision, or overall EOS, whichever occurs first.
For patients who were not eligible for study treatment with [¹⁷⁷Lu]Lu-DPI-4452 but received [⁶⁸Ga]Ga-DPI-4452, EOS will be 7 days after the [⁶⁸Ga]Ga-DPI-4452 injection.
Overall EOS will take place when all patients have completed a minimum of 12 months follow-up after their last dose, have discontinued prematurely, or if the study is terminated prematurely, whichever occurs first. At this timepoint (±2 months), all patients (who have not previously discontinued from the study prematurely) will undergo the EOS assessments and will be considered as having completed the study.

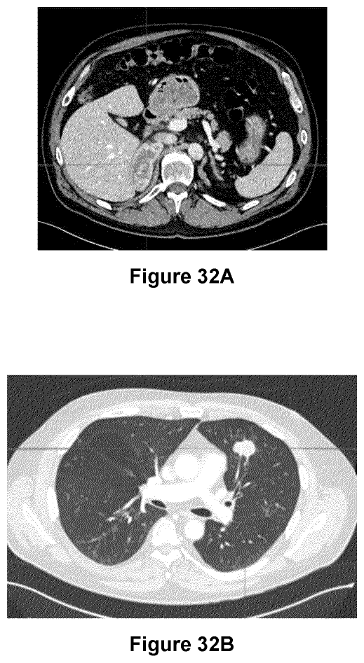

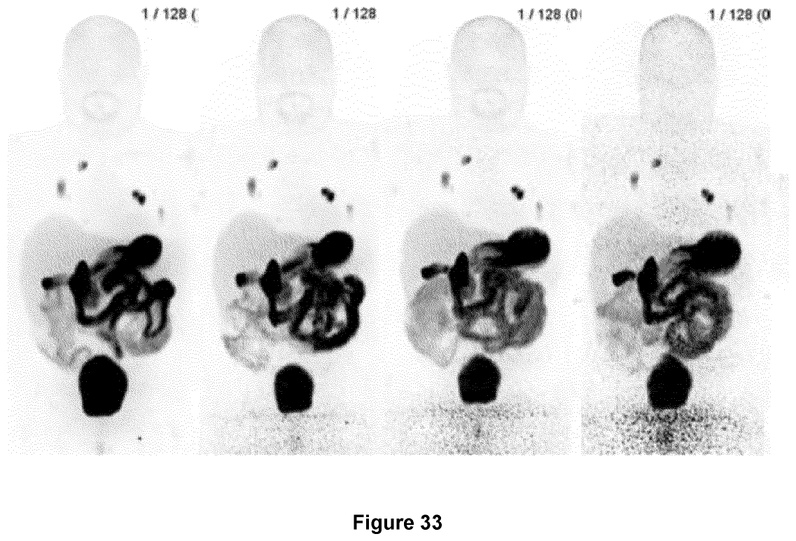

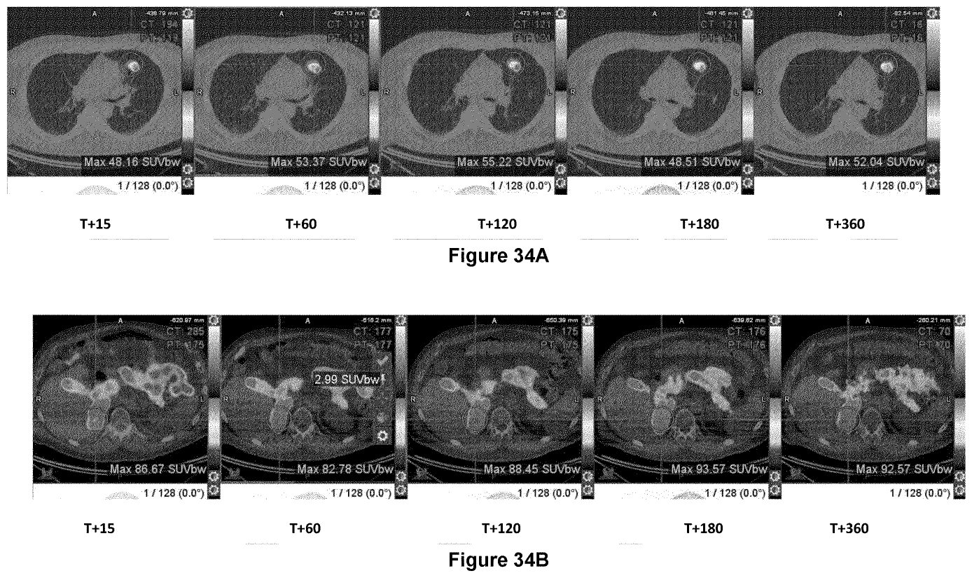

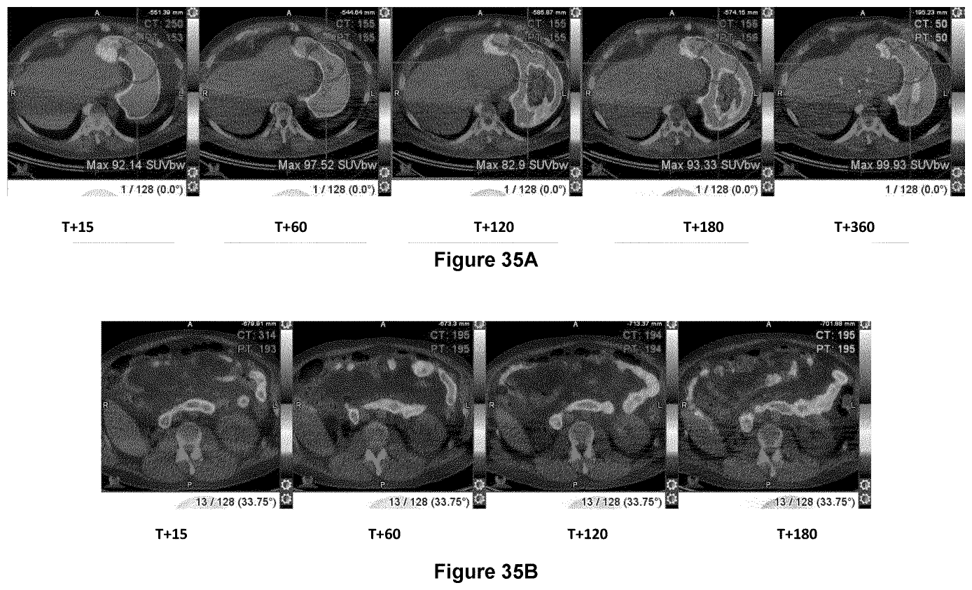

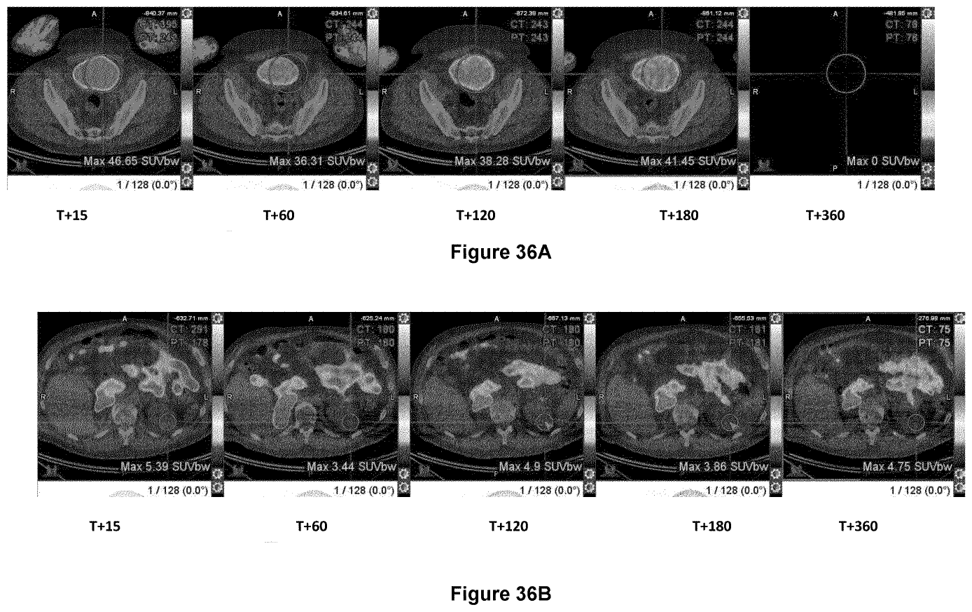

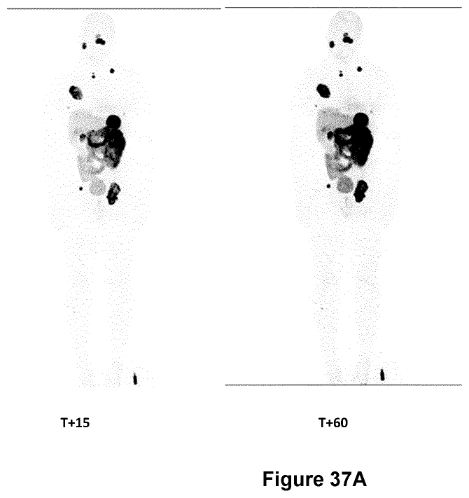

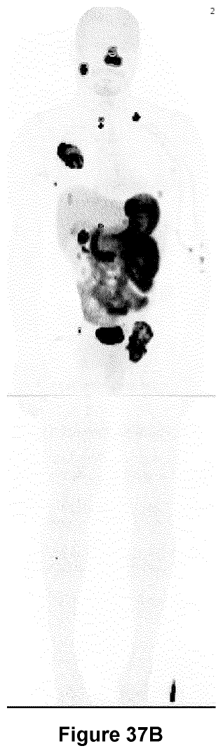

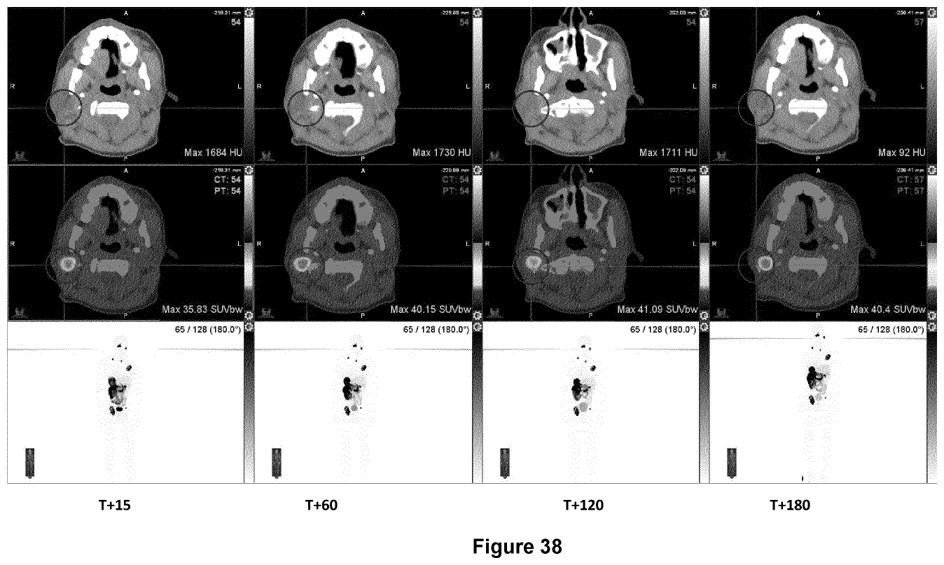

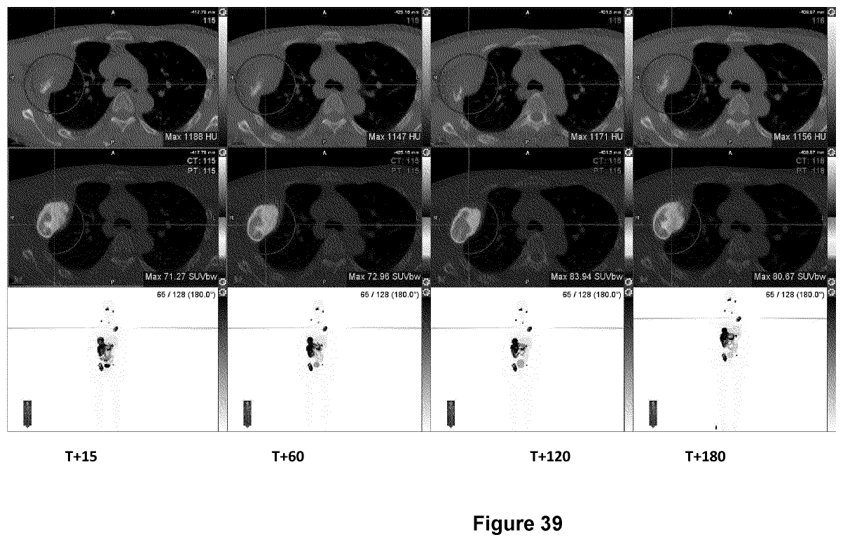

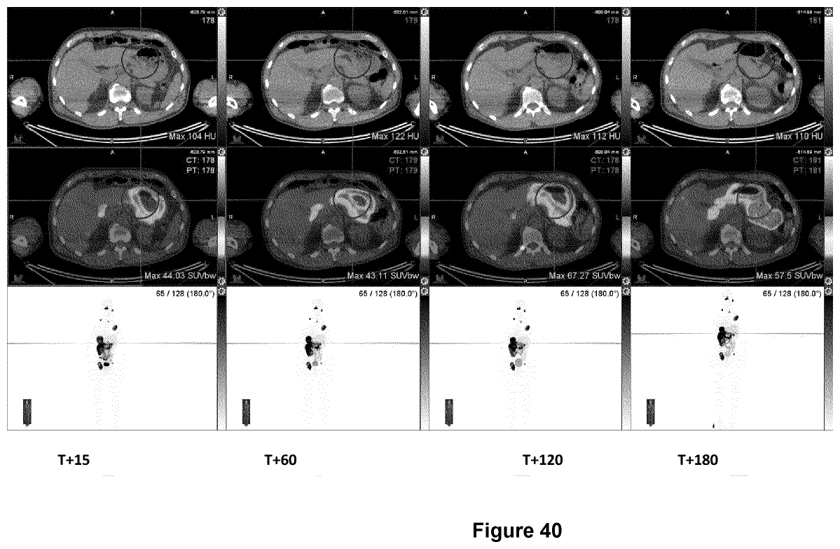

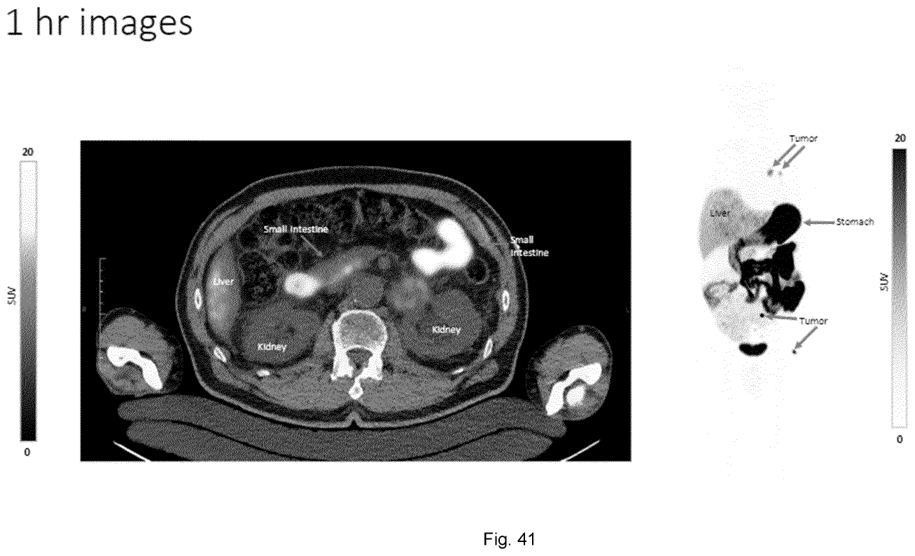

PET imaging results in human patients with CAIX positive disease (metastatic ccRCC) after a single 185 MBq dose of Ga⁶⁸-DPI-4452, are shown in table 38 and FIGS. 32 to 36B for patient 1 and 37A to 40 for patient 2.

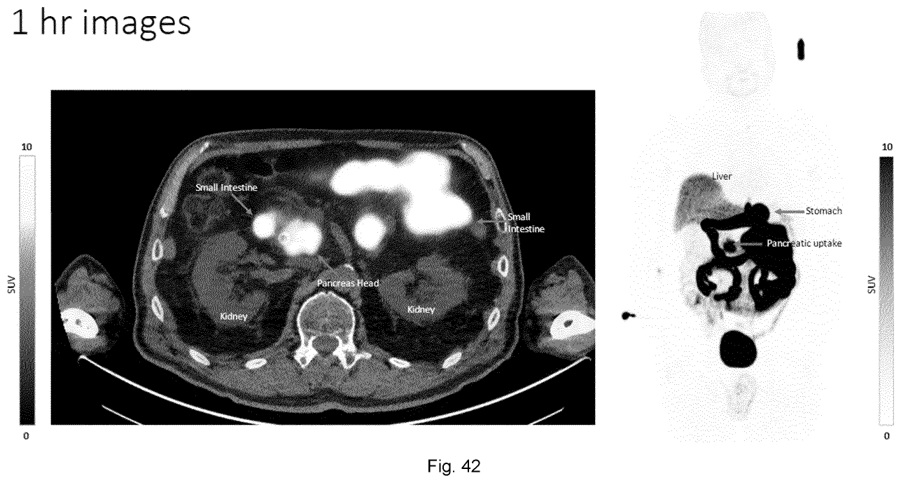

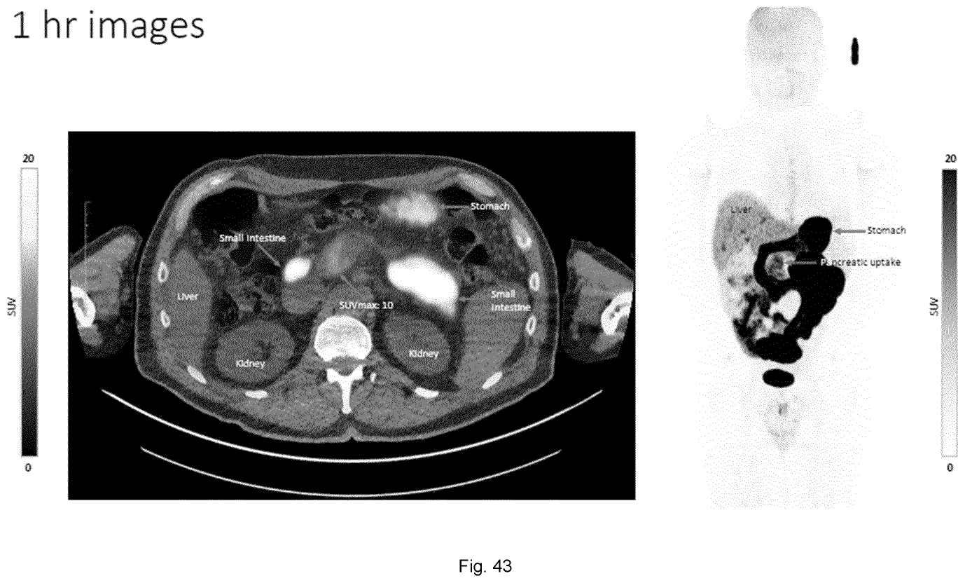

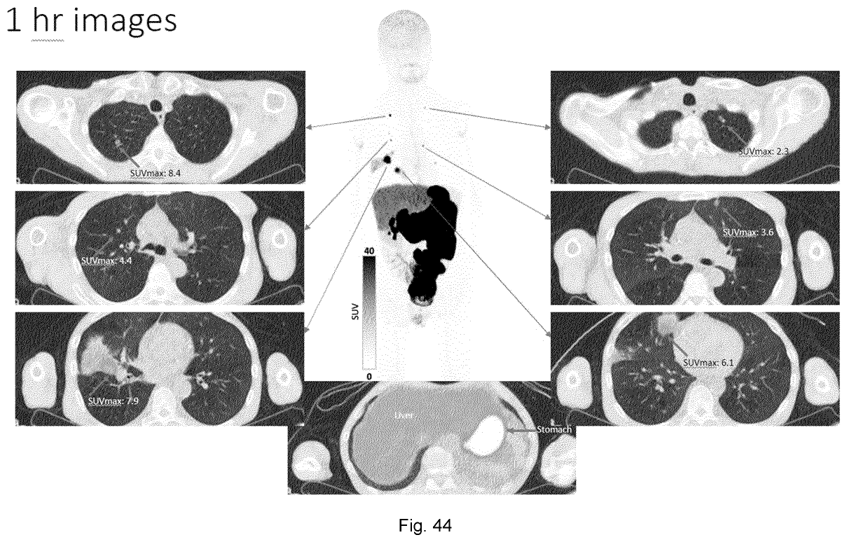

Imaging results in human patients with CAIX positive disease (PDAC (Patients 3, 4 and 5), and 1×CRC (patient 6)) after a single 185 MBq dose of Ga68-DPI-4452, are shown in table 39 and FIGS. 41 to 44.

TABLE 38
Ga⁶⁸-DPI-4452 CT and PET imaging analysis in
patients with CAIX positive disease (ccRCC)

| | Patient 1 | Patient 2 |
|---|---|---|
| Lesions detected by both CT and PET | 4 | 6 |
| Discordant lesions (not detected by PET) | 1 | 0 |
| Lesions found on PET only | 0 | 8 |
| Lesion SUVmax range | 7 to 38 | 7 to 160 |

TABLE 39
Ga⁶⁸-DPI-4452 CT and PET imaging analysis in patients
with CAIX positive disease (PDAC or CRC)

| | Lesions detection: | Lesion SUVmax |
|---|---|---|
| Patient | Concordance PET/CT | range |
| 3 | 100% | 9.1 to 33.7 |
| 4 | 100% | 14.7 |
| 5 | 50% | 10.4 |
| 6 | 60% | 2.39-8.39 |

Imaging results show:
The outstanding imaging properties of Ga⁶⁸-DPI-4452,
The specific and rapid high tumor uptake of Ga⁶⁸-DPI-4452
Early bladder uptake and rapid excretion in urine,
Very low kidney, liver, and spleen uptake
Concordance between CAIX protein expression (as measured by immunohistochemistry) and Ga⁶⁸ uptake.

1. A method of treating a carbonic anhydrase IX (CAIX) positive disease in a human patient, the method comprising the steps of:
(i) optionally administering to the patient an imaging dose of a compound labeled with ⁶⁸Ga, to obtain an image of body parts or tissues to be examined; and
(ii) administering to the patient a treatment dose of the compound labeled with ¹⁷⁷Lu, to treat the CAIX positive disease;
wherein the compound is represented by the following Formula (1): 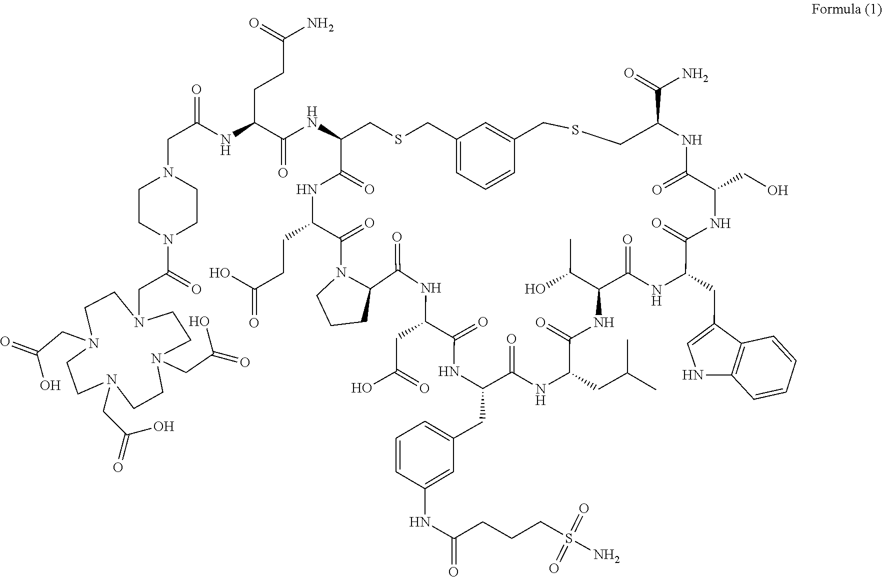 [SMILES: CC(C)C[C@@H]1NC(=O)[C@H](Cc2cccc(NC(=O)CCCS(N)(=O)=O)c2)NC(=O)[C@H](CC(=O)O)NC(=O)[C@H]2CCCN2C(=O)[C@H](CCC(=O)O)NC(=O)[C@@H](NC(=O)[C@H](CCC(N)=O)NC(=O)CN2CCN(C(=O)CN3CCN(CC(=O)O)CCN(CC(=O)O)CCN(CC(=O)O)CC3)CC2)CSCc2cccc(c2)CSC[C@@H](C(N)=O)NC(=O)[C@H](CO)NC(=O)[C@H](Cc2c[nH]c3ccccc23)NC(=O)[C@H]([C@@H](C)O)NC1=O]
wherein the DOTA moiety comprised in the compound of Formula (1) chelates the ⁶⁸Ga or ¹⁷⁷Lu to form the compound labeled with ⁶⁸Ga or ¹⁷⁷Lu;
wherein, if the patient is not administered an imaging dose of the compound labeled with ⁶⁸Ga in step (i), the compound labeled with ¹⁷⁷Lu administered in step (ii) is administered once per cycle of one to six weeks.

2. The method according to claim 1, wherein said method comprises the steps of:
(i) administering to the patient an imaging dose of the compound labeled with ⁶⁸Ga, to obtain an image of body parts or tissues to be examined, and
(ii) administering to the patient a treatment dose of the compound labeled with ¹⁷⁷Lu, to treat the CAIX positive disease.

3. The method according to claim 1, wherein:
(a) the imaging dose of the compound labeled with ⁶⁸Ga is from 50 to 250 MBq; and/or
(b) the treatment dose of the compound labeled with ¹⁷⁷Lu is from 1.0 to 25.0 GBq.

4. The method according to claim 1, wherein the compound labeled with ¹⁷⁷Lu is administered once per cycle of one to six weeks.

5. The method according to claim 1, wherein the compound labeled with ¹⁷⁷Lu-labeled is administered over one to ten cycles.

6. The method according to claim 1, wherein the treatment dose of the compound labeled with ¹⁷⁷Lu is administered in at least one cycle, and the treatment dose is selected from the following:
(1) a treatment dose of from 2.0 to 6.0 GBq,
(2) a treatment dose of from 6.0 to 10.0 GBq,
(3) a treatment dose of from 10.0 to 14.0 GBq,
(4) a treatment dose of from 14.0 to 18.0 GBq, and
(5) a treatment dose of from 18.0 to 20.0 GBq.

7. The method according to claim 1, wherein the compound labeled with ⁶⁸Ga and/or the compound labeled with ¹⁷⁷Lu is administered intravenously.

8. The method according to claim 1, wherein the compound labeled with ⁶⁸Ga and/or the compound labeled with ¹⁷⁷Lu is provided as a solution in a pharmaceutically acceptable injectable carrier.

9. The method according to claim 8, wherein the solution of the compound labeled with ⁶⁸Ga has a concentration of from 250 to 950 MBq/8.1 mL, and the solution of the compound labeled with ¹⁷⁷Lu has a concentration of from 150 to 900 MBq/mL.

10. The method according to claim 1, wherein the CAIX positive disease is cancer.

11. The method according to claim 1, wherein the CAIX positive disease is a cancer selected from the group consisting of renal cell carcinoma (RCC), clear cell renal cell carcinoma (ccRCC), colorectal carcinoma (CRC), pancreatic ductal adenocarcinoma (PDAC), glioblastoma (GBM), mesothelioma, cholangiocarcinoma (CCA), ovarian carcinoma, non-small cell lung cancer (NSCLC), squamous non-small cell lung cancer (SNSCLC), brain cancer, pancreatic cancer, thyroid cancer, lung cancer, renal cancer, breast cancer, triple-negative breast cancer (TNBC), head and neck cancer, squamous carcinoma of head and neck (SCCHN), urothelial carcinoma and bladder cancer.

12. The method according to claim 1, wherein the CAIX positive disease is a cancer selected from the group consisting of ccRCC, CRC, PDAC, SNSCLC, TNBC and SCCHN.

13. The method according to claim 1, wherein the compound labeled with ⁶⁸Ga and/or the compound labeled with ¹⁷⁷Lu is administered after one or more other therapeutic agents or therapies, selected from DNA damage response (DDR) inhibitors, chemotherapeutic agents, immunomodulatory agents, proton pump inhibitors (PPIs), histamine H₂-receptor antagonists, tyrosine kinase inhibitors, cell therapy, external beam radiation, and any other targeted therapies.

14. The method according to claim 1, wherein the compound labeled with ¹⁷⁷Lu administered in step (ii) is administered once per cycle of four to six weeks.

15. The method according to claim 1, wherein the compound labeled with ¹⁷⁷Lu administered in step (ii) is administered once per cycle of four weeks.

16. The method according to claim 3, wherein:
(a) the imaging dose of the compound labeled with ⁶⁸Ga is from 100 to 200 MBq; and/or
(b) the treatment dose of the compound labeled with ¹⁷⁷Lu is from 2.0 to 20.0 GBq.

17. The method according to claim 1, wherein the treatment dose of the compound labeled with ¹⁷⁷Lu that is administered in at least one cycle, and the treatment dose is selected from the following:
(1) a treatment dose of about 3.7 GBq,
(2) a treatment dose of about 7.4 GBq,
(3) a treatment dose of about 11.1 GBq,
(4) a treatment dose of about 14.8 GBq, and
(5) a treatment dose of about 18.5 GBq.

18. The method according to claim 1, wherein the compound labeled with ¹⁷⁷Lu-labeled is administered over four to eight cycles.

19. The method according to claim 1, wherein the compound labeled with ⁶⁸Ga and/or the compound labeled with ¹⁷⁷Lu is administered intravenously by infusion.

20. The method according to claim 1, wherein the CAIX positive disease is cancer, and the human patient has unresectable, locally advanced, or metastatic solid tumors.
